(19) United States
(12) Patent Application Publication
(10) Pub. No.: US 2011/0301143 A1
(43) Pub. Date: Dec. 8, 2011

(54) HETEROCYCLIC DERIVATIVES AS INHIBITORS OF STEAROYL-COENZYME A DELTA-9 DESATURASE

(76) Inventors: Elise Isabel, Pointe-Claire (CA); Nicolas Lachance, Pierrefonds (CA); Jean-Philippe Leclerc, Laval (CA); Serge Leger, Notre-Dame-de-I'lle-Perrol (CA); Renata M. Oballa, Kirkland (CA); David Powell, Verdun (CA); Yeeman K. Ramtohul, Pierrefonds (CA); Patrick Roy, Dollard-des-Ormeaux (CA); Geoffrey K. Tranmer, Hamilton (CA); Renee Aspiotis, Kirkland (CA); Lianhai Li, Pierrefonds (CA); Evelyn Martins, Vaudreuil-Dorion (CA)

(21) Appl. No.: 13/201,720

(22) PCT Filed: Feb. 18, 2010
(86) PCT No.: PCT/CA2010/000228

§ 371 Date: Aug. 16, 2011

(30) Foreign Application Priority Data
Feb. 23, 2009 (CA) 61208337
Jul. 30, 2009 (CA) 61229835

(51) Int. Cl. A61K 31/497; C07D 417/14; C07D 413/14; C07D 401/14; A61K 31/506; A61K 31/496; A61K 31/427; A61K 31/454; A61K 31/4439; A61K 31/4995; A61K 31/551; A61P 3/10; A61P 5/48; A61P 3/00; A61P 3/06; A61P 7/00; C07D 403/14

(52) U.S. Cl. 514/210.18; 544/357; 544/295; 544/369; 544/367; 548/194; 546/209; 546/270.4; 544/331; 544/349; 540/575; 514/252.11; 514/252.19; 514/254.02; 514/254.03; 514/254.04; 514/326; 514/342; 514/275; 514/249; 514/218

(57) ABSTRACT

Heterocyclic compounds of structural formula (I), or a pharmaceutically acceptable salt thereof, wherein W is a R¹— substituted heteroaryl, R¹ is an heteroaryl ring substituted with an ester or carboxylic acid containing radical, X-T is N—CR⁵R⁶, C═CR⁵ or CR¹³—CR⁵R⁶, Y is a bond or —C(O)—, a and b represent an integer selected from 1 to 4, and Ar is an optionally substituted phenyl or naphtyl, are inhibitors of stearoyl-coenzyme A delta-9 desaturase (SCD) The heterocyclic compounds are useful for the prevention and treatment of conditions related to abnormal lipid synthesis and metabolism, including cardiovascular disease, atherosclerosis, obesity, diabetes, neurological disease, Metabolic Syndrome, insulin resistance, cancer, liver steatosis, and non-alcoholic steatohepatitis.

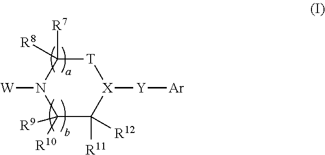

### FIELD OF THE INVENTION

The present invention relates to heterocyclic derivatives which are inhibitors of stearoyl-coenzyme A delta-9 desaturase (SCD) and the use of such compounds to control, prevent and/or treat conditions or diseases mediated by SCD activity. The compounds of the present invention are useful for the control, prevention and treatment of conditions and diseases related to abnormal lipid synthesis and metabolism, including cardiovascular disease; atherosclerosis; obesity; diabetes; neurological disease; Metabolic Syndrome; insulin resistance; cancer; liver steatosis; and non-alcoholic steatohepatitis.

### BACKGROUND OF THE INVENTION

At least three classes of fatty acyl-coenzyme A (CoA) desaturases (delta-5, delta-6 and delta-9 desaturases) are responsible for the formation of double bonds in mono- and polyunsaturated fatty acyl-CoAs derived from either dietary sources or de novo synthesis in mammals. The delta-9 specific stearoyl-CoA desaturases (SCDs) catalyze the rate-limiting formation of the cis-double bond at the C9-C10 position in monounsaturated fatty acyl-CoAs. The preferred substrates are stearoyl-CoA and palmitoyl-CoA, with the resulting oleoyl and palmitoleoyl-CoA as the main components in the biosynthesis of phospholipids, triglycerides, cholesterol esters and wax esters (Dobrzyn and Natami, Obesity Reviews, 6: 169-174 (2005)).

The rat liver microsomal SCD protein was first isolated and characterized in 1974 (Strittmatter et al., PNAS, 71: 4565-4569 (1974)). A number of mammalian SCD genes have since been cloned and studied from various species. For example, two genes have been identified from rat (SCD1 and SCD2, Thiede et al., J. Biol. Chem., 261, 13230-13235 (1986)), Mihara, K., J. Biochem. (Tokyo), 108: 1022-1029 (1990)); four genes from mouse (SCD1, SCD2, SCD3 and SCD4) (Miyazaki et al., J. Biol. Chem., 278: 33904-33911 (2003)); and two genes from human (SCD1 and ACOD4 (SCD2)), (Zhang, et al., Biochem. J., 340: 255-264 (1991); Beiraghi, et al., Gene, 309: 11-21 (2003); Zhang et al., Biochem. J., 388: 135-142 (2005)). The involvement of SCDs in fatty acid metabolism has been known in rats and mice since the 1970's (Oshino, N., Arch. Biochem. Biophys., 149: 378-387 (1972)). This has been further supported by the biological studies of a) Asebia mice that carry the natural mutation in the SCD1 gene (Zheng et al., Nature Genetics, 23: 268-270 (1999)), b) SCD1-null mice from targeted gene deletion (Ntambi, et al., PNAS, 99: 11482-11486 (2002), and c) the suppression of SCD1 expression during leptin-induced weight loss (Cohen et al., Science, 297: 240-243 (2002)). The potential benefits of pharmacological inhibition of SCD activity has been demonstrated with anti-sense oligonucleotide inhibitors (ASO) in mice (Jiang, et al., J. Clin. Invest., 115: 1030-1038 (2005)). ASO inhibition of SCD activity reduced fatty acid synthesis and increased fatty acid oxidation in primary mouse hepatocytes. Treatment of mice with SCD-ASOs resulted in the prevention of diet-induced obesity, reduced body adiposity, hepatomegaly, steatosis, postprandial plasma insulin and glucose levels, reduced de novo fatty acid synthesis, decreased the expression of lipogenic genes, and increased the expression of genes promoting energy expenditure in liver and adipose tissues. Thus, SCD inhibition represents a novel therapeutic strategy in the treatment of obesity and related metabolic disorders.

There is compelling evidence to support that elevated SCD activity in humans is directly implicated in several common disease processes. For example, there is an elevated hepatic lipogenesis to triglyceride secretion in non-alcoholic fatty liver disease patients (Diraison, et al., Diabetes Metabolism, 29: 478-485 (2003)); Donnelly, et al., J. Clin. Invest., 115: 1343-1351 (2005)). Elevated SCD activity in adipose tissue is closely coupled to the development of insulin resistance (Sjogren, et al., Diabetologia, 51(2): 328-35 (2007)). The postprandial de novo lipogenesis is significantly elevated in obese subjects (Marques-Lopes, et al., American Journal of Clinical Nutrition, 73: 252-261 (2001)). Knockout of the SCD gene ameliorates Metabolic Syndrome by reducing plasma triglycerides, reducing weight gain, increasing insulin sensitivity, and reduces hepatic lipid accumulation (MacDonald, et al., Journal of Lipid Research, 49(1): 217-29 (2007)). There is a significant correlation between a high SCD activity and an increased cardiovascular risk profile including elevated plasma triglycerides, a high body mass index and reduced plasma HDL (Attie, et al., J. Lipid Res., 43: 1899-1907 (2002)). SCD activity plays a key role in controlling the proliferation and survival of human transformed cells (Scaglia and Igal, J. Biol. Chem., (2005)). RNA interference of SCD-1 reduces human tumor cell survival (Morgan-Lappe, et al., Cancer Research, 67(9): 4390-4398 (2007)).

Other than the above mentioned anti-sense oligonucleotides, inhibitors of SCD activity include non-selective thia-fatty acid substrate analogs [B. Behrouzian and P. H. Buist, Prostaglandins, Leukotrienes, and Essential Fatty Acids, 68: 107-112 (2003)], cyclopropenoid fatty acids (Raju and Reiser, J. Biol. Chem., 242: 379-384 (1967)), certain conjugated long-chain fatty acid isomers (Park, et al., Biochim. Biophys. Acta, 1486: 285-292 (2000)), and a series of heterocyclic derivatives disclosed in published international patent application publications WO 2005/011653, WO 2005/011654, WO 2005/011656, WO 2005/011656, WO 2005/011657, WO 2006/014168, WO 2006/034279, WO 2006/034312, WO 2006/034315, WO 2006/034338, WO 2006/034341, WO 2006/034440, WO 2006/034441, WO 2006/034446, WO 2006/086445; WO 2006/086447; WO 2006/101521; WO 2006/125178; WO 2006/125179; WO 2006/125180; WO 2006/125181; WO 2006/125194; WO 2007/044085; WO 2007/046867; WO 2007/046868; WO 2007/050124; WO 2007/130075; WO 2007/136746; WO 2008/036715; WO 2008/074835; WO 2008/127349; and U.S. Pat. Nos. 7,456,180 and 7,390,813; all assigned to Xenon Pharmaceuticals, Inc. or Xenon Pharmaceuticals, Inc./Novartis AG.

A number of international patent applications assigned to Merck Frosst Canada Ltd. that disclose SCD inhibitors useful for the treatment of obesity and Type 2 diabetes have also published: WO 2006/130986 (14 Dec. 2006); WO 2007/009236 (25 Jan. 2007); WO 2007/056846 (24 May 2007); WO 2007/071023 (28 Jun. 2007); WO 2007/134457 (29 Nov. 2007); WO 2007/143823 (21 Dec. 2007); WO 2007/143824 (21 Dec. 2007); WO 2008/017161 (14 Feb. 2008); WO 2008/046226 (24 Apr. 2008); WO 2008/064474 (5 Jun. 2008); WO 2008/089580 (31 Jul. 2008); WO 2008/128335 (30 Oct. 2008); WO 2008/141455 (27 Nov. 2008); US 2008/0132542 (5 Jun. 2008); and US 2008/0182838 (31 Jul. 2008).

WO 2008/003753 (assigned to Novartis) discloses a series of pyrazolo[1,5-c]pyrimidine analogs as SCD inhibitors; WO 2007/143597 and WO 2008/024390 (assigned to Novartis AG and Xenon Pharmaceuticals) disclose heterocyclic derivatives as SCD inhibitors; and WO 2008/096746 (assigned to Takeda Pharmaceutical) disclose Spiro compounds as SCD inhibitors.

Additional international patent applications disclosing SCD inhibitors have published: WO 2008/062276 (Glenmark; 29 May 2008); WO 2008 (Glenmark; 13 Mar. 2008); WO 2008/003753 (Biovitrum AB; 10 Jan. 2008); WO 2008/135141 (Sanofi-Aventis; 13 Nov. 2008); WO 2008/157844 (Sanofi-Aventis; 24 Dec. 2008); WO 2008/104524 (SKB; 4 Sep. 2008); WO 2008/074834 (SKB; 26 Jun. 2008); WO 2008/074833 (SKB; 26 Jun. 2008); WO 2008/074832 (SKB; 26 Jun. 2008); and WO 2008/074824 (SKB; 26 Jun. 2008).

Small molecule SCD inhibitors have also been described by (a) G. Liu, et al., “Discovery of Potent, Selective, Orally Bioavailable SCD1 Inhibitors,” in J. Med. Chem., 50: 3086-3100 (2007); (b) H. Zhao, et al., “Discovery of 1-(4-phenoxypiperidin-1-yl)-2-arylaminoethanone SCD 1 inhibitors,” Bioorg. Med. Chem. Lett., 17: 3388-3391 (2007); and (c) Z. Xin, et al., “Discovery of piperidine-aryl urea-based stearoyl-CoA desaturase 1 inhibitors,” Bioorg. Med. Chem. Lett., 18: 4298-4302 (2008).

The present invention is concerned with novel heteroaromatic compounds as inhibitors of stearoyl-CoA delta-9 desaturase which are useful in the treatment and/or prevention of various conditions and diseases mediated by SCD activity including those related, but not limited, to elevated lipid levels, as exemplified in non-alcoholic fatty liver disease, cardiovascular disease, obesity, diabetes, metabolic syndrome, and insulin resistance.

The role of stearoyl-coenzyme A desaturase in lipid metabolism has been described by M. Miyazaki and J. M. Ntambi, Prostaglandins, Leukotrienes, and Essential Fatty Acids, 68: 113-121 (2003). The therapeutic potential of the pharmacological manipulation of SCD activity has been described by A. Dobrzyn and J. M. Ntambi, in “Stearoyl-CoA desaturase as a new drug target for obesity treatment,” Obesity Reviews, 6: 169-174 (2005).

### SUMMARY OF THE INVENTION

The present invention relates to heterocyclic derivatives of structural formula I:

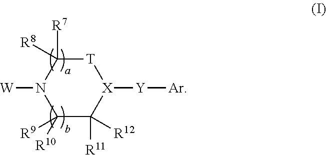

These heterocyclic derivatives are effective as inhibitors of SCD. They are therefore useful for the treatment, control or prevention of disorders responsive to the inhibition of SCD, such as diabetes, insulin resistance, lipid disorders, obesity, atherosclerosis, and metabolic syndrome.

The present invention also relates to pharmaceutical compositions comprising the compounds of the present invention and a pharmaceutically acceptable carrier.

The present invention also relates to methods for the treatment, control, or prevention of disorders, diseases, or conditions responsive to inhibition of SCD in a subject in need thereof by administering the compounds and pharmaceutical compositions of the present invention.

The present invention also relates to methods for the treatment, control, or prevention of Type 2 diabetes, insulin resistance, obesity, lipid disorders, atherosclerosis, and metabolic syndrome by administering the compounds and pharmaceutical compositions of the present invention.

The present invention also relates to methods for the treatment, control, or prevention of obesity by administering the compounds of the present invention in combination with a therapeutically effective amount of another agent known to be useful to treat the condition.

The present invention also relates to methods for the treatment, control, or prevention of Type 2 diabetes by administering the compounds of the present invention in combination with a therapeutically effective amount of another agent known to be useful to treat the condition.

The present invention also relates to methods for the treatment, control, or prevention of atherosclerosis by administering the compounds of the present invention in combination with a therapeutically effective amount of another agent known to be useful to treat the condition.

The present invention also relates to methods for the treatment, control, or prevention of lipid disorders by administering the compounds of the present invention in combination with a therapeutically effective amount of another agent known to be useful to treat the condition.

The present invention also relates to methods for treating metabolic syndrome by administering the compounds of the present invention in combination with a therapeutically effective amount of another agent known to be useful to treat the condition.

### DETAILED DESCRIPTION OF THE INVENTION

The present invention is concerned with heterocyclic derivatives useful as inhibitors of SCD. Compounds of the present invention are described by structural formula I:

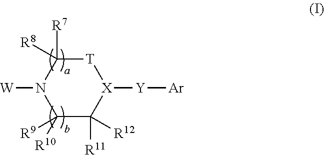

and pharmaceutically acceptable salts thereof; wherein
“a” is an integer selected from 0, 1, and 2;
“b” is an integer selected from 0, 1, and 2;
with the proviso that “a” and “b” cannot both be 2;

### X-T is N—CR⁵R⁶, C═CR⁵, or CR¹³—CR⁵R⁶;

Y is a bond or C(═O);
W is heteroaryl selected from the group consisting of:

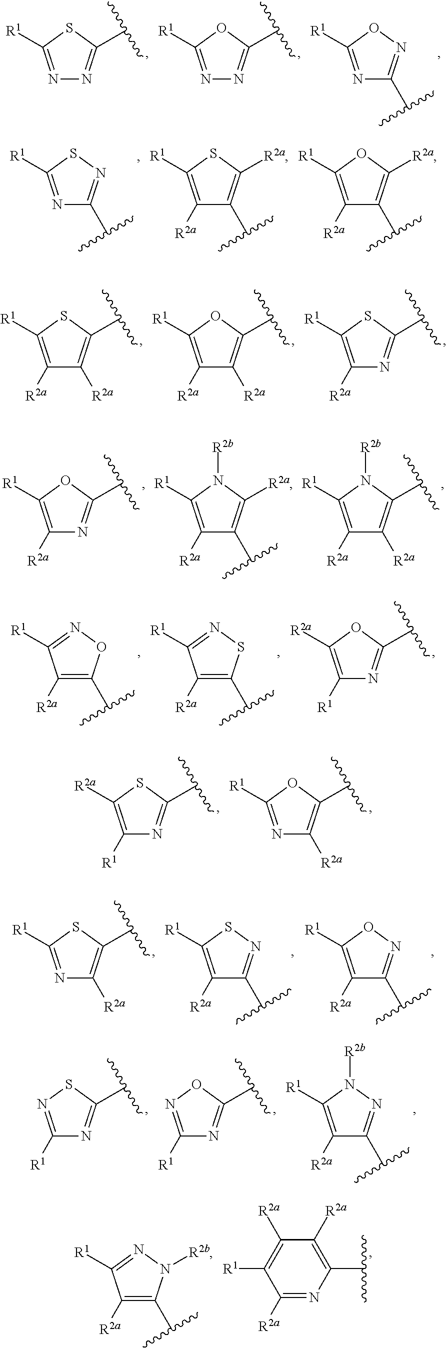

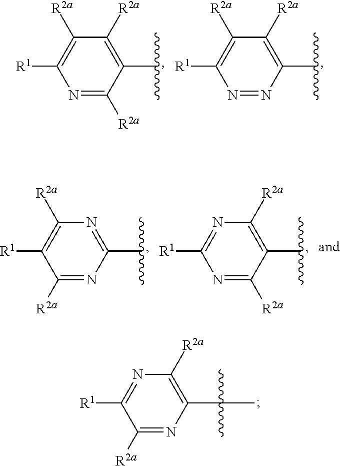

R¹ is heteroaryl selected from the group consisting of:

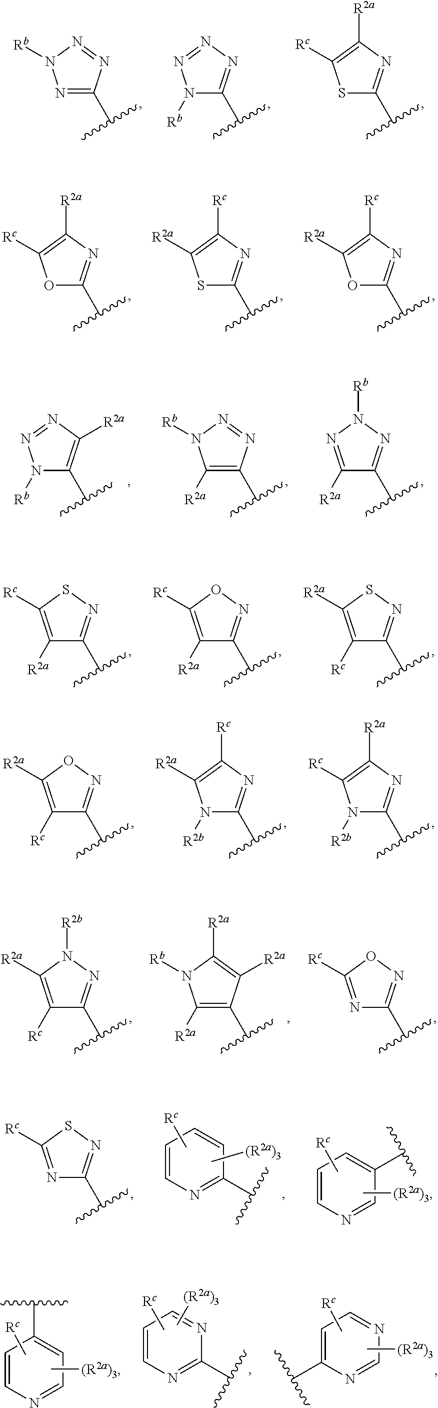

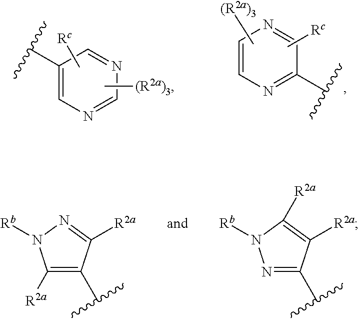

wherein
R^{b} is —(CH₂)ᵣCO₂H, —(CH₂)ᵣCO₂C₁₋₃ alkyl, —(CH₂)ᵣ—Z—(CH₂)ₚCO₂H, or —(CH₂)ᵣZ—(CH₂)ₚCO₂Cl_{—}3 alkyl;
R^{c} is —(CH₂)ₘCO₂H, —(CH₂)ₘCO₂C₁₋₃ alkyl, —(CH₂)ₘ—Z—(CH₂)ₚCO₂H, or —(CH₂)ₘ—Z—(CH₂)ₚCO₂C₁₋₃ alkyl;

### Z is O, S, or NR⁴;

each R^{2a} is independently selected from the group consisting of:

hydrogen,

halogen,

hydroxy,

cyano,

amino,

C₁₋₄ alkyl, optionally substituted with one to five fluorines,

C₁₋₄ alkoxy, optionally substituted with one to five fluorines,

C₁₋₄ alkylthio, optionally substituted with one to five fluorines,

C₁₋₄ alkylsulfonyl, optionally substituted with one to five fluorines,

carboxy,

C₁₋₄ alkyloxycarbonyl, and

C₁₋₄ alkylcarbonyl;

each R^{2b} is independently selected from the group consisting of:

hydrogen,

C₁₋₄ alkyl, optionally substituted with one to five fluorines,

C₁₋₄ alkylsulfonyl, optionally substituted with one to five fluorines,

C₁₋₄ alkyloxycarbonyl, and

C₁₋₄ alkylcarbonyl;

Ar is phenyl, naphthyl, thienyl, or pyridyl optionally substituted with one to five R³ substituents;
each R³ is independently selected from the group consisting of:

halogen,

cyano,

C₁₋₆ alkyl, optionally substituted with one to five fluorines,

C₃₋₅ cycloalkyl,

C₃₋₅ cycloalkylmethyl, optionally substituted with C₁₋₃ alkyl,

C₁₋₆ alkoxy, optionally substituted with one to five fluorines,

C₁₋₆ alkylthio, optionally substituted with one to five fluorines, and

C₁₋₆ alkylsulfonyl, optionally substituted with one to five fluorines;

each R⁴ is independently selected from the group consisting of hydrogen,

C₁₋₆ alkyl,

(CH₂)ₙ-phenyl,

(CH₂)ₙ-heteroaryl,

(CH₂)ₙ-naphthyl, and

(CH₂)ₙC₃₋₇ cycloalkyl;

wherein alkyl, phenyl, heteroaryl, naphthyl, and cycloalkyl are optionally substituted with one to three groups independently selected from halogen, C₁₋₄ alkyl, and C₁₋₄ alkoxy;
R⁵, R⁶, R⁷, R⁸, R⁹, R¹⁰, R¹¹, and R¹² are each independently hydrogen, fluorine, or C₁₋₃ alkyl, wherein alkyl is optionally substituted with one to three substituents independently selected from fluorine and hydroxy;
or one of R⁵, R⁶, R⁷, and R⁸ together with one of R⁹, R¹⁰, R¹¹, and R¹² forms a direct bond or a C₁₋₂ alkylene bridge;
R¹³ is hydrogen, C₁₋₃ alkyl, fluorine, or hydroxy;
m is an integer from 0 to 3;
n is an integer from 0 to 2;
p is an integer from 1 to 3; and
r is an integer from 1 to 3.

In one embodiment of the compounds of the present invention, “a” and “b” are each 1, to give a 6-membered piperidine ring system. In a class of this first embodiment, X-T is CR¹³—CR⁵R⁶; and Y is a bond. In a subclass of this class, R⁵, R⁶, and R¹³ are each hydrogen.

In a second class of this first embodiment, X-T is CR¹³—CR⁵R⁶; and Y is C(═O). In a subclass of this class, R⁵, R⁶, and R¹³ are each hydrogen.

In a third class of this first embodiment, X-T is N—CR⁵R⁶; and Y is a bond. In a subclass of this class, R⁵ and R⁶ are each hydrogen. In another subclass of this class, one of R⁵, R⁶, R⁷, and R⁸ together with one of R⁹, R¹⁰, R¹¹, and R¹² forms a methylene bridge.

In a fourth class of this first embodiment, X-T is N—CR⁵R⁶; and Y is C(═O). In a subclass of this class, R⁵ and R⁶ are each hydrogen. In another subclass of this class, one of R⁵, R⁶, R⁷, and R⁸ together with one of R⁹, R¹⁰, R¹¹, and R¹² forms a methylene bridge.

In a fifth class of this first embodiment, X-T is C═CR⁵; and Y is a bond. In a subclass of this class, R⁵ is hydrogen.

In a second embodiment of the compounds of the present invention, “a” and “b” are each 0, to give a 4-membered azetidine ring system. In a class of this second embodiment, X-T is CR¹³—CR⁵R⁶; and Y is a bond. In a subclass of this class, R⁵, R⁶, and R¹³ are each hydrogen.

In a second class of this second embodiment, X-T is CR¹³—CR⁵R⁶; and Y is C(═O). In a subclass of this class, R⁵, R⁶, and R¹³ are each hydrogen.

In a third class of this second embodiment, X-T is N—CR⁵R⁶; and Y is a bond. In a subclass of this class, R⁵ and R⁶ are each hydrogen.

In a fourth class of this second embodiment, X-T is N—CR⁵R⁶; and Y is C(═O). In a subclass of this class, R⁵ and R⁶ are each hydrogen.

In a fifth class of this second embodiment, X-T is C═CR⁵; and Y is a bond. In a subclass of this class, R⁵ is hydrogen.

In a third embodiment of the compounds of the present invention, “a” is 1 and “b” is 2, to give a 7-membered azepine ring system. In a class of this third embodiment, X-T is CR¹³—CR⁵R⁶; and Y is a bond. In a subclass of this class, R⁵, R⁶, and R¹³ are each hydrogen.

In a second class of this third embodiment, X-T is CR¹³—CR⁵R⁶; and Y is C(═O). In a subclass of this class, R⁵, R⁶, and R¹³ are each hydrogen.

In a third class of this third embodiment, X-T is N—CR⁵R⁶; and Y is a bond. In a subclass of this class, R⁵ and R⁶ are each hydrogen.

In a fourth class of this third embodiment, X-T is N—CR⁵R⁶; and Y is C(═O). In a subclass of this class, R⁵ and R⁶ are each hydrogen.

In a fifth class of this third embodiment, X-T is C═CR⁵; and Y is a bond. In a subclass of this class, R⁵ is hydrogen.

In a fourth embodiment of the compounds of the present invention, “a” is 2 and “b” is 1, to give a 7-membered azepine ring system. In a class of this fourth embodiment, X-T is CR¹³—CR⁵R⁶; and Y is a bond. In a subclass of this class, R⁵, R⁶, and R¹³ are each hydrogen.

In a second class of this fourth embodiment, X-T is CR¹³—CR⁵R⁶; and Y is C(═O). In a subclass of this class, R⁵, R⁶, and R¹³ are each hydrogen.

In a third class of this fourth embodiment, X-T is N—CR⁵R⁶; and Y is a bond. In a subclass of this class, R⁵ and R⁶ are each hydrogen.

In a fourth class of this fourth embodiment, X-T is N—CR⁵R⁶; and Y is C(═O). In a subclass of this class, R⁵ and R⁶ are each hydrogen.

In a fifth class of this fourth embodiment, X-T is C═CR⁵; and Y is a bond. In a subclass of this class, R⁵ is hydrogen.

In a fifth embodiment of the compounds of the present invention, Ar is phenyl optionally substituted with one to three substituents independently selected from R³ as defined above. In a class of this fifth embodiment, R³ is halogen, trifluoromethyl, or trifluoromethoxy.

In a sixth embodiment of the compounds of the present invention, R⁵, R⁶, R⁷, R⁸, R⁹, R¹⁰, R¹¹, R¹² and R¹³ are each hydrogen.

In a seventh embodiment of the compounds of the present invention, W is heteroaryl selected from the group consisting of:

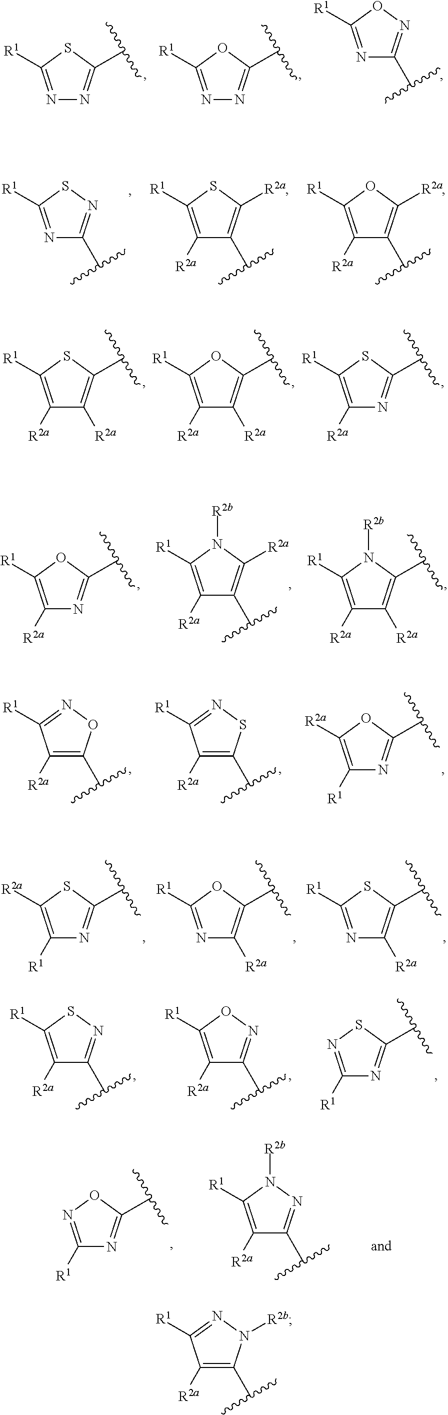

wherein R¹ and R^{2a} are as defined above. In a class of this embodiment, R^{2a} and R^{2b} are each hydrogen.

In another class of this seventh embodiment, W is heteroaryl selected from the group consisting of:

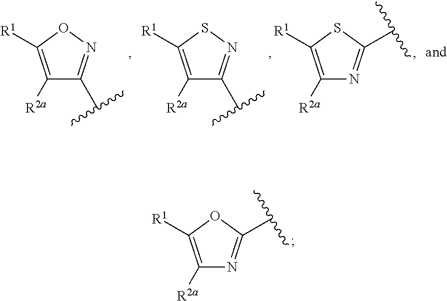

wherein R¹ and R^{2a} are as defined above. In a subclass of this class, R^{2a} is hydrogen.

In an eighth embodiment of the compounds of the present invention, W is heteroaryl selected from the group consisting of:

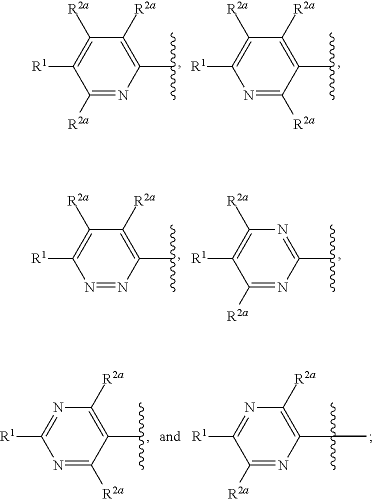

wherein R¹ and R^{2a} are as defined above. In a class of this embodiment, each R^{2a} is hydrogen. In another class of this embodiment, W is

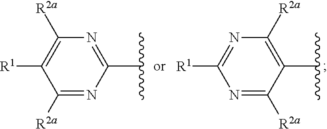

wherein R¹ and R^{2a} are as defined above. In a subclass of this class, each R^{2a} is hydrogen.

In a ninth embodiment of the compounds of the present invention, R¹ is heteroaryl selected from the group consisting of:

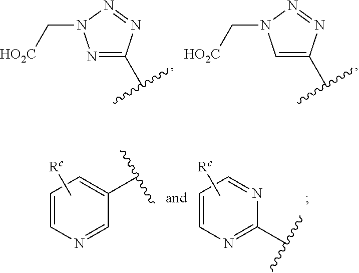

wherein R^{c} is —CO₂H, —CO₂C₁₋₃ alkyl, —CH₂CO₂H, or —CH₂CO₂C₁₋₃ alkyl. In a class of this ninth embodiment, R¹ is

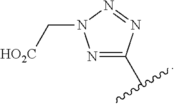

In a tenth embodiment of the compounds of the present invention, W is heteroaryl selected from the group consisting of:

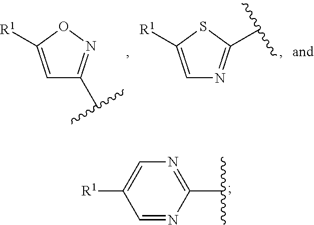

and R¹ is heteroaryl selected from the group consisting of:

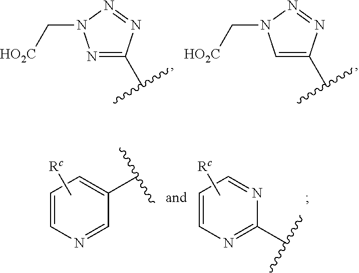

wherein R^{c} is —CO₂H, —CO₂C₁₋₃ alkyl, —CH₂CO₂H, or —CH₂CO₂C₁₋₃ alkyl.

In a class of this tenth embodiment, W is

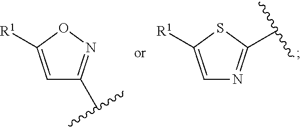

### and R¹ is

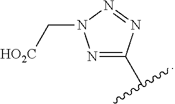

In an eleventh embodiment of the compounds of the present invention,

“a” and “b” are each 1;

### X-T is CH—CH₂;

Y is a bond;
R⁷, R⁸, R⁹, R¹⁰, R¹¹, and R¹² are each hydrogen;
Ar is phenyl optionally substituted with one to three substituents independently selected from halogen, trifluoromethyl, and trifluoromethoxy;
W is heteroaryl selected from the group consisting of:

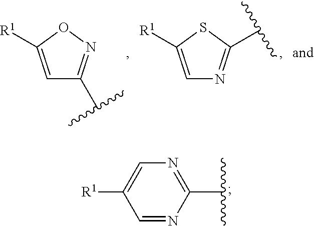

and R¹ is heteroaryl selected from the group consisting of:

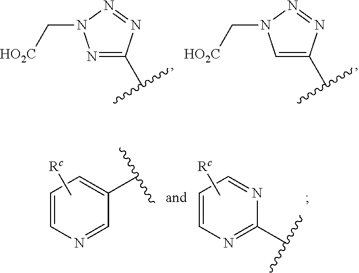

wherein R^{c} is —CO₂H, —CO₂C₁₋₃ alkyl, —CH₂CO₂H, or —CH₂CO₂C₁₋₃ alkyl.

In a class of this eleventh embodiment, W is

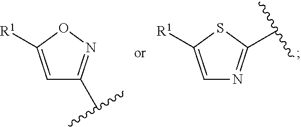

### and R¹ is

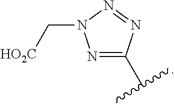

In a twelfth embodiment of the compounds of the present invention,

“a” and “b” are each 1;

### X-T is CH—CH₂;

### Y is C(═O);

R⁷, R⁸, R⁹, R¹⁰, R¹¹, and R¹² are each hydrogen;
Ar is phenyl optionally substituted with one to three substituents independently selected from halogen, trifluoromethyl, and trifluoromethoxy;
W is heteroaryl selected from the group consisting of:

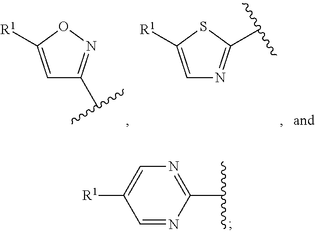

and R¹ is heteroaryl selected from the group consisting of:

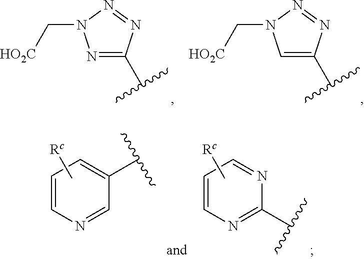

wherein R^{c} is —CO₂H, —CO₂C₁₋₃ alkyl, —CH₂CO₂H, or —CH₂CO₂C₁₋₃ alkyl.

In a class of this twelfth embodiment, W is

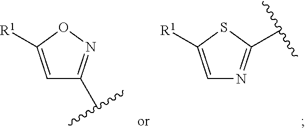

### and R¹ is

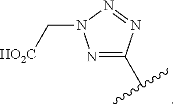

In a thirteenth embodiment of the compounds of the present invention,

“a” and “b” are each 1;

### X-T is N—CH₂;

Y is a bond;
R⁷, R⁸, R⁹, R¹⁰, R¹¹, and R¹² are each hydrogen;
Ar is phenyl optionally substituted with one to three substituents independently selected from halogen, trifluoromethyl, and trifluoromethoxy;
W is heteroaryl selected from the group consisting of:

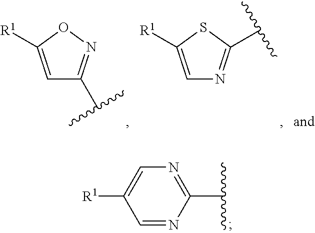

and R¹ is heteroaryl selected from the group consisting of:

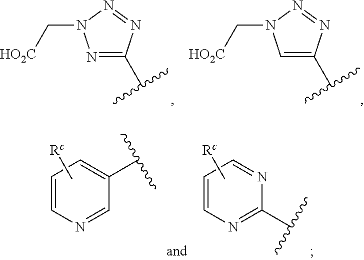

wherein R^{c} is —CO₂H, —CO₂C₁₋₃ alkyl, —CH₂CO₂H, or —CH₂CO₂C₁₋₃ alkyl.

In a class of this thirteenth embodiment, W is

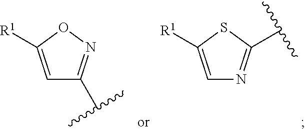

### and R¹ is

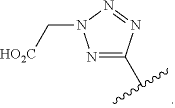

In a fourteenth embodiment of the compounds of the present invention,

“a” and “b” are each 1;

### X-T is N—CH₂;

### Y is C(═O);

R⁷, R⁸, R⁹, R¹⁰, R¹¹, and R¹² are each hydrogen;
Ar is phenyl optionally substituted with one to three substituents independently selected from halogen, trifluoromethyl, and trifluoromethoxy;
W is heteroaryl selected from the group consisting of:

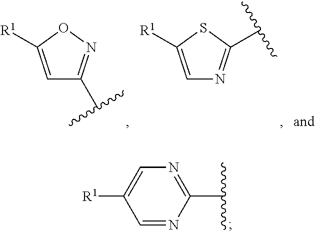

and R¹ is heteroaryl selected from the group consisting of:

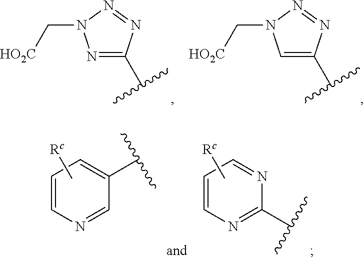

wherein R^{c} is —CO₂H, —CO₂C₁₋₃ alkyl, —CH₂CO₂H, or —CH₂CO₂C₁₋₃ alkyl.

In a class of this fourteenth embodiment, W is

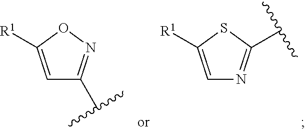

### and R¹ is

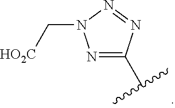

In a fifteenth embodiment of the compounds of the present invention,

“a” and “b” are each 1;

### X-T is CH═CH;

Y is a bond;
R⁷, R⁸, R⁹, R¹⁰, R¹¹, and R¹² are each hydrogen;
Ar is phenyl optionally substituted with one to three substituents independently selected from halogen, trifluoromethyl, and trifluoromethoxy;
W is heteroaryl selected from the group consisting of:

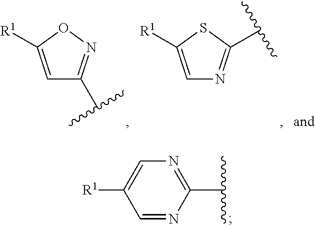

and R¹ is heteroaryl selected from the group consisting of:

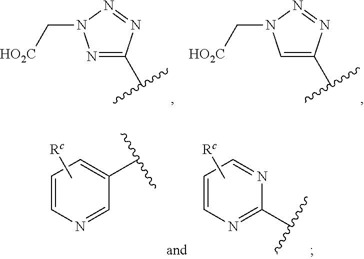

wherein R^{c} is —CO₂H, —CO₂C₁₋₃ alkyl, —CH₂CO₂H, or —CH₂CO₂C₁₋₃ alkyl.

In a class of this fifteenth embodiment, W is

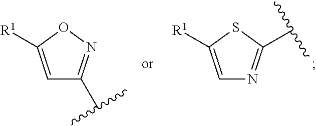

### and R¹ is

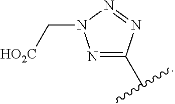

In a sixteenth embodiment of the compounds of the present invention,

“a” is 2 and “b” is 1;

### X-T is N—CH₂;

Y is a bond;
R⁷, R⁸, R⁹, R¹⁰, R¹¹, and R¹² are each hydrogen;
Ar is phenyl optionally substituted with one to three substituents independently selected from halogen, trifluoromethyl, and trifluoromethoxy;
W is heteroaryl selected from the group consisting of:

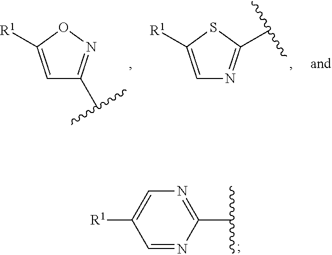

and R¹ is heteroaryl selected from the group consisting of:

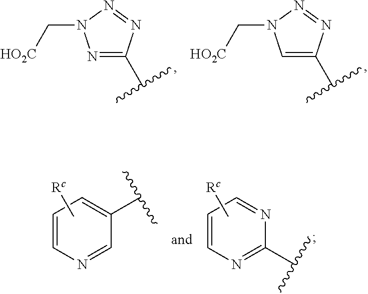

wherein R^{c} is —CO₂H, —CO₂C₁₋₃ alkyl, —CH₂CO₂H, or —CH₂CO₂C₁₋₃ alkyl.

In a class of this sixteenth embodiment, W is

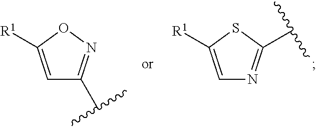

### and R¹ is

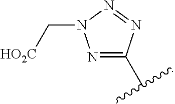

Illustrative, but nonlimiting, examples of compounds of the present invention that are useful as inhibitors of human SCD-1 are the following:

| Example | IC₅₀ hSCD-1 |
|---|---|
| 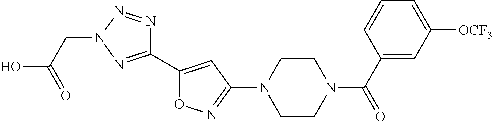 | <20 nM |
| 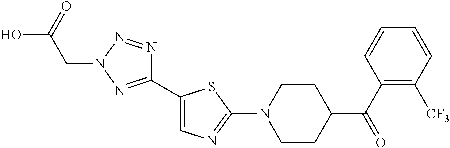 | <20 nM |
| 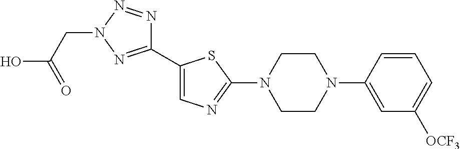 | <20 nM |
| 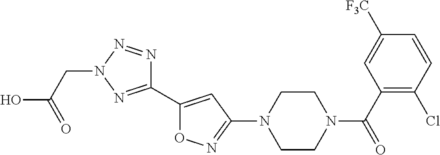 | 24 nM |
| 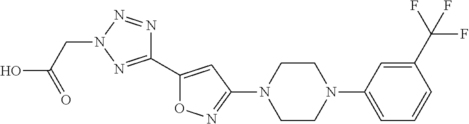 | 83 nM |
| 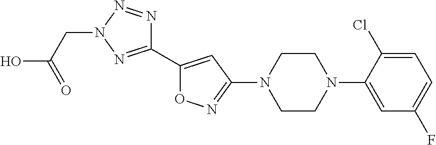 | 35 nM |
| 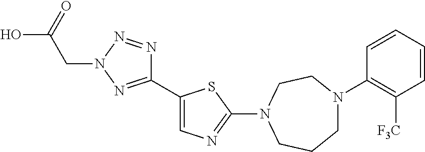 | 30 nM |
| 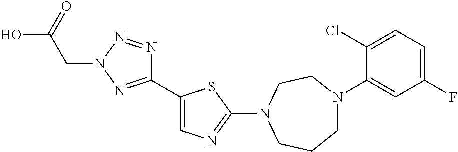 | 29 nM |
| 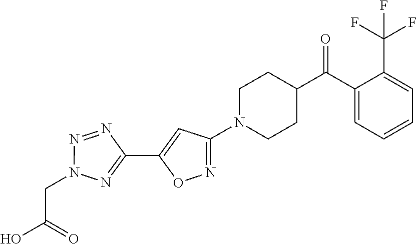 | <20 nM |
| 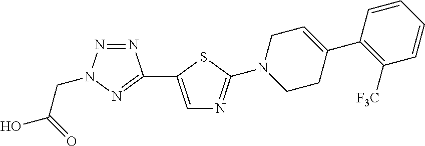 | <20 nM |
| 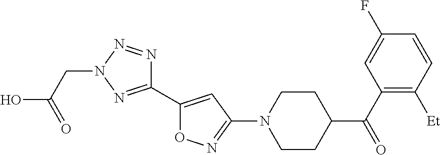 | <20 nM |
| 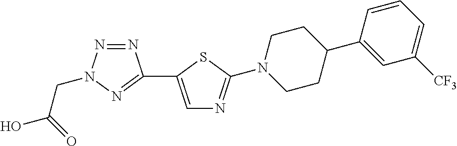 | <20 nM |
| 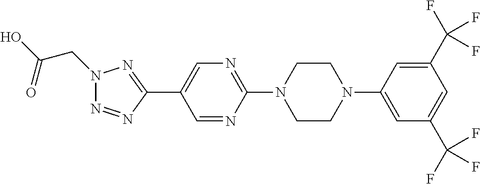 | 44 nM |
| 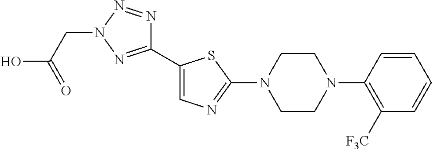 | <20 nM |
| 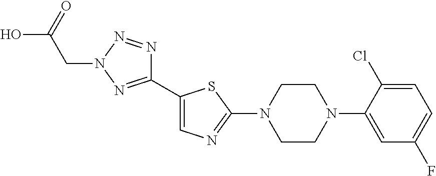 | <20 nM |
| 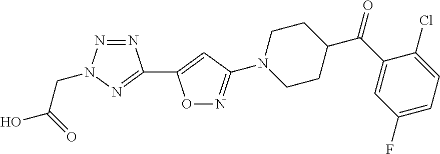 | <20 nM |
| 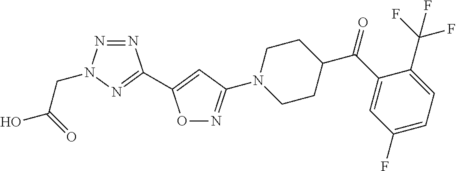 | <20 nM |
| 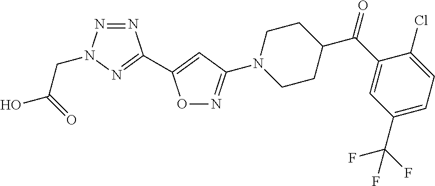 | 22 nM |
| 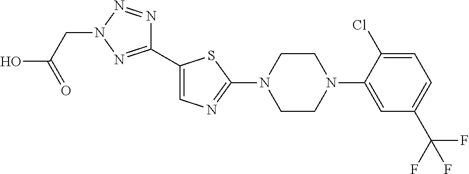 | <20 nM |
| 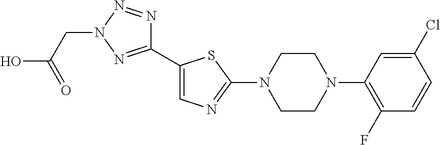 | <20 nM |
| 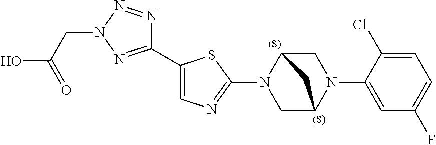 | <20 nM |
| 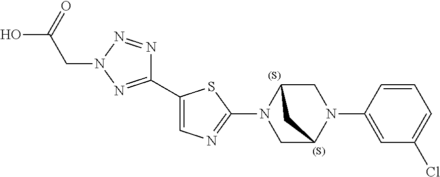 | <20 nM |
| 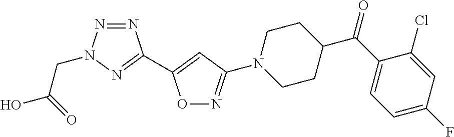 | 50 nM |
| 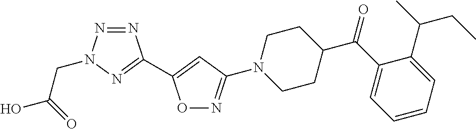 | <20 nM |
| 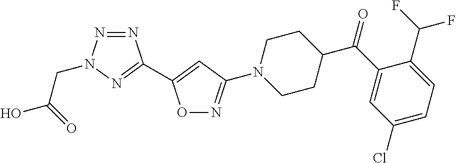 | <20 nM |
| 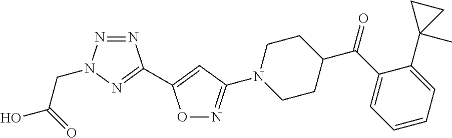 | <20 nM |
| 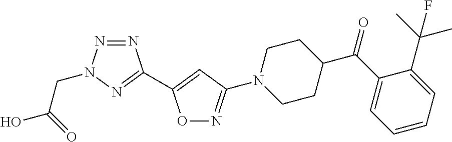 | <20 nM |
| 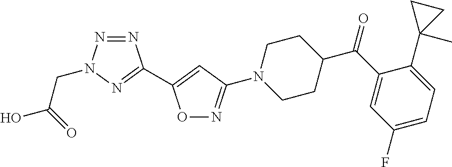 | <20 nM |
| 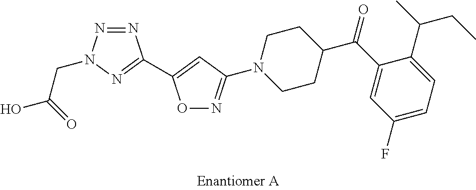 | <20 nM |
| 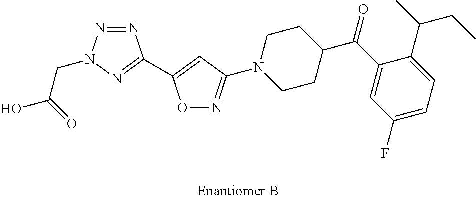 | <20 nM |
| 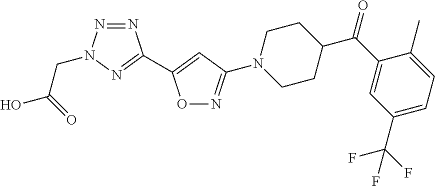 | 48 nM |
| 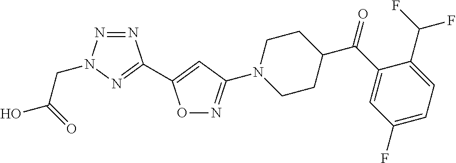 | <20 nM |
| 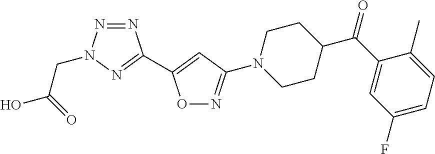 | 20 nM |
| 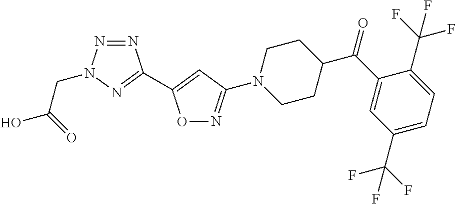 | <20 nM |
| 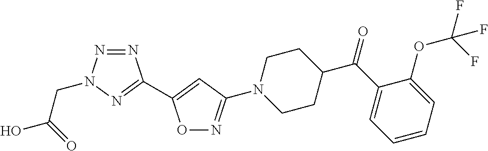 | 66 nM |
| 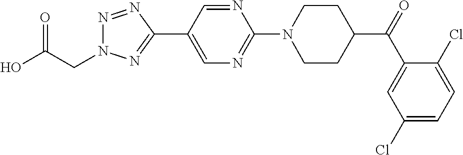 | 58 nM |
| 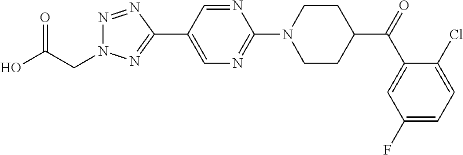 | 46 nM |
| 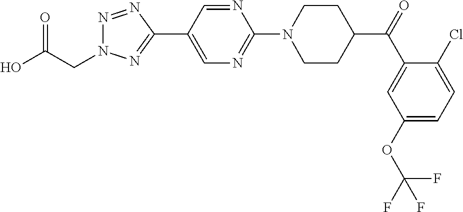 | <20 nM |
| 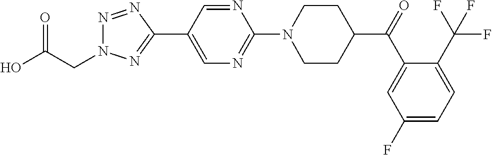 | 27 nM |
| 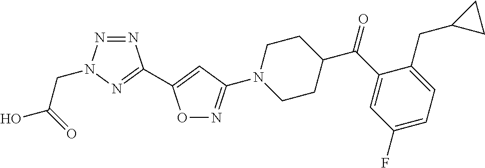 | <20 nM |
| 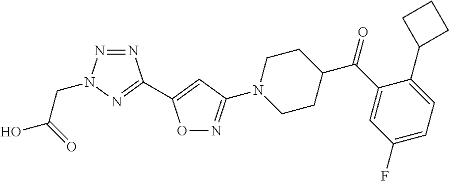 | <20 nM |
| 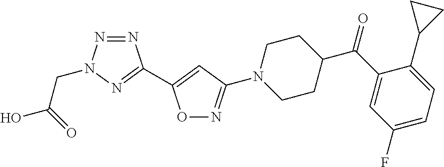 | <20 nM |
| 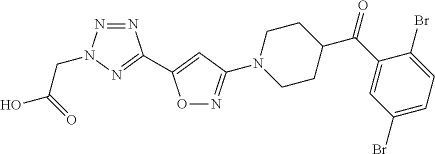 | <20 nM |
| 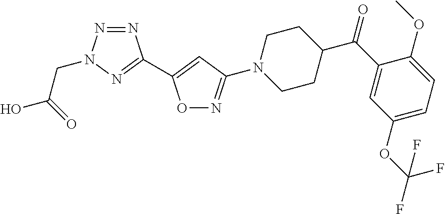 | 85 nM |
| 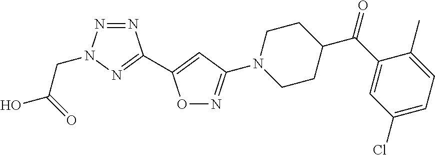 | 28 nM |
| 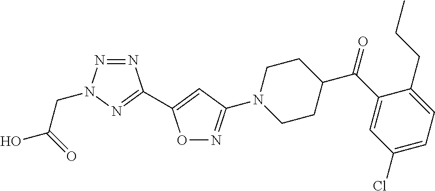 | 23 nM |
| 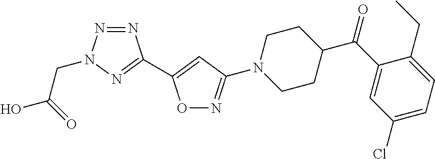 | <20 nM |
| 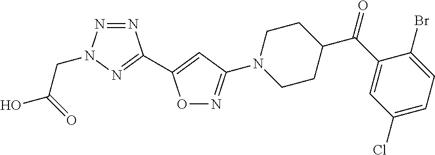 | <20 nM |
| 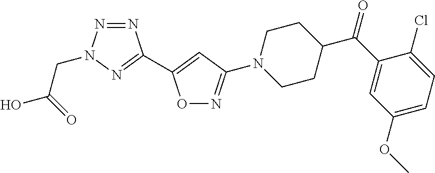 | 95 nM |
| 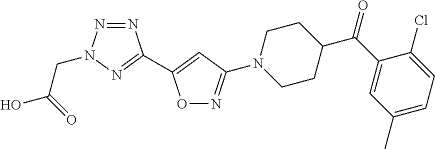 | 79 nM |
| 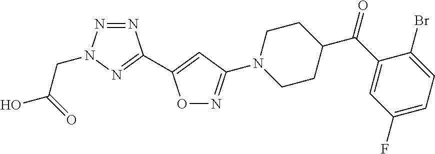 | <20 nM |
| 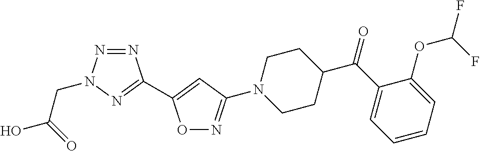 | 40 nM |
| 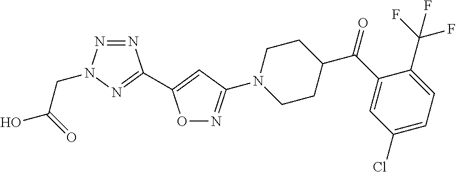 | 32 nM |
| 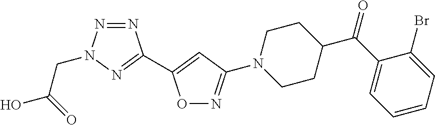 | 28 nM |
| 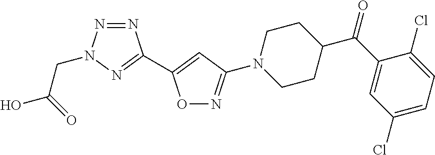 | 31 nM |

and pharmaceutically acceptable salts thereof.

As used herein the following definitions are applicable.

“Alkyl”, as well as other groups having the prefix “alk”, such as alkoxy and alkanoyl, means carbon chains which may be linear or branched, and combinations thereof, unless the carbon chain is defined otherwise. Examples of alkyl groups include methyl, ethyl, propyl, isopropyl, butyl, sec- and tert-butyl, pentyl, hexyl, heptyl, octyl, nonyl, and the like. Where the specified number of carbon atoms permits, e.g., from C₃₋₁₀, the term alkyl also includes cycloalkyl groups, and combinations of linear or branched alkyl chains combined with cycloalkyl structures. When no number of carbon atoms is specified, C₁₋₆ is intended.

“Cycloalkyl” is a subset of alkyl and means a saturated carbocyclic ring having a specified number of carbon atoms. Examples of cycloalkyl include cyclopropyl, cyclobutyl, cyclopentyl, cyclohexyl, cycloheptyl, cyclooctyl, and the like. A cycloalkyl group generally is monocyclic unless stated otherwise. Cycloalkyl groups are saturated unless otherwise defined.

The term “alkoxy” refers to straight or branched chain alkoxides of the number of carbon atoms specified (e.g., C₁₋₆ alkoxy), or any number within this range [i.e., methoxy (MeO—), ethoxy, isopropoxy, etc.].

The term “alkylthio” refers to straight or branched chain alkylsulfides of the number of carbon atoms specified (e.g., C₁₋₆ alkylthio), or any number within this range [i.e., methylthio (MeS—), ethylthio, isopropylthio, etc.].

The term “alkylamino” refers to straight or branched alkylamines of the number of carbon atoms specified (e.g., C₁₋₆ alkylamino), or any number within this range [i.e., methylamino, ethylamino, isopropylamino, t-butylamino, etc.].

The term “alkylsulfonyl” refers to straight or branched chain alkylsulfones of the number of carbon atoms specified (e.g., C₁₋₆ alkylsulfonyl), or any number within this range [i.e., methylsulfonyl (MeSO₂—), ethylsulfonyl, isopropylsulfonyl, etc.].

The term “alkylsulfinyl” refers to straight or branched chain alkylsulfoxides of the number of carbon atoms specified (e.g., C₁₋₆ alkylsulfinyl), or any number within this range [i.e., methylsulfinyl (MeSO—), ethylsulfinyl, isopropylsulfinyl, etc.].

The term “alkyloxycarbonyl” refers to straight or branched chain esters of a carboxylic acid derivative of the present invention of the number of carbon atoms specified (e.g., C₁₋₆ alkyloxycarbonyl), or any number within this range [i.e., methyloxycarbonyl (MeOCO—), ethyloxycarbonyl, or butyloxycarbonyl].

“Aryl” means a mono- or polycyclic aromatic ring system containing carbon ring atoms. The preferred aryls are monocyclic or bicyclic 6-10 membered aromatic ring systems. Phenyl and naphthyl are preferred aryls. The most preferred aryl is phenyl.

“Heterocyclyl” refer to saturated or unsaturated non-aromatic rings or ring systems containing at least one heteroatom selected from O, S and N, further including the oxidized forms of sulfur, namely SO and SO₂. Examples of heterocycles include tetrahydrofuran (THF), dihydrofuran, 1,4-dioxane, morpholine, 1,4-dithiane, piperazine, piperidine, 1,3-dioxolane, imidazolidine, imidazoline, pyrroline, pyrrolidine, tetrahydropyran, dihydropyran, oxathiolane, dithiolane, 1,3-dioxane, 1,3-dithiane, oxathiane, thiomorpholine, 2-oxopiperidin-1-yl, 2-oxopyrrolidin-1-yl, 2-oxoazetidin-1-yl, 1,2,4-oxadiazin-5(6H)-one-3-yl, and the like.

“Heteroaryl” means an aromatic or partially aromatic heterocycle that contains at least one ring heteroatom selected from O, S and N. Heteroaryls thus includes heteroaryls fused to other kinds of rings, such as aryls, cycloalkyls and heterocycles that are not aromatic. Examples of heteroaryl groups include: pyrrolyl, isoxazolyl, isothiazolyl, pyrazolyl, pyridyl, oxazolyl, oxadiazolyl (in particular, 1,3,4-oxadiazol-2-yl and 1,2,4-oxadiazol-3-yl), thiadiazolyl, thiazolyl, imidazolyl, triazolyl, tetrazolyl, furyl, triazinyl, thienyl, pyrimidyl, benzisoxazolyl, benzoxazolyl, benzothiazolyl, benzothiadiazolyl, dihydrobenzofuranyl, indolinyl, pyridazinyl, indazolyl, isoindolyl, dihydrobenzothienyl, indolizinyl, cinnolinyl, phthalazinyl, quinazolinyl, naphthyridinyl, carbazolyl, benzodioxolyl, quinoxalinyl, purinyl, furazanyl, isobenzylfuranyl, benzimidazolyl, benzofuranyl, benzothienyl, quinolyl, indolyl, isoquinolyl, dibenzofuranyl, and the like. For heterocyclyl and heteroaryl groups, rings and ring systems containing from 3-15 atoms are included, forming 1-3 rings.

“Halogen” refers to fluorine, chlorine, bromine and iodine. Chlorine and fluorine are generally preferred. Fluorine is most preferred when the halogens are substituted on an alkyl or alkoxy group (e.g. CF₃O and CF₃CH₂O).

Compounds of structural formula I may contain one or more asymmetric centers and can thus occur as racemates and racemic mixtures, single enantiomers, diastereomeric mixtures and individual diastereomers. The present invention is meant to comprehend all such isomeric forms of the compounds of structural formula I.

Compounds of structural formula I may be separated into their individual diastereoisomers by, for example, fractional crystallization from a suitable solvent, for example methanol or ethyl acetate or a mixture thereof, or via chiral chromatography using an optically active stationary phase. Absolute stereochemistry may be determined by X-ray crystallography of crystalline products or crystalline intermediates which are derivatized, if necessary, with a reagent containing an asymmetric center of known absolute configuration.

Alternatively, any stereoisomer of a compound of the general structural formula I may be obtained by stereospecific synthesis using optically pure starting materials or reagents of known absolute configuration.

If desired, racemic mixtures of the compounds may be separated so that the individual enantiomers are isolated. The separation can be carried out by methods well known in the art, such as the coupling of a racemic mixture of compounds to an enantiomerically pure compound to form a diastereomeric mixture, followed by separation of the individual diastereomers by standard methods, such as fractional crystallization or chromatography. The coupling reaction is often the formation of salts using an enantiomerically pure acid or base. The diasteromeric derivatives may then be converted to the pure enantiomers by cleavage of the added chiral residue. The racemic mixture of the compounds can also be separated directly by chromatographic methods utilizing chiral stationary phases, which methods are well known in the art.

Some of the compounds described herein contain olefinic double bonds, and unless specified otherwise, are meant to include both E and Z geometric isomers.

Some of the compounds described herein may exist as tautomers, which have different points of attachment of hydrogen accompanied by one or more double bond shifts. For example, a ketone and its enol form are keto-enol tautomers. The individual tautomers as well as mixtures thereof are encompassed with compounds of the present invention.
In the compounds of generic Formula I, the atoms may exhibit their natural isotopic abundances, or one or more of the atoms may be artificially enriched in a particular isotope having the same atomic number, but an atomic mass or mass number different from the atomic mass or mass number predominantly found in nature. The present invention is meant to include all suitable isotopic variations of the compounds of generic Formula I. For example, different isotopic forms of hydrogen (H) include protium (¹H) and deuterium (²H). Protium is the predominant hydrogen isotope found in nature. Enriching for deuterium may afford certain therapeutic advantages, such as increasing in vivo half-life or reducing dosage requirements, or may provide a compound useful as a standard for characterization of biological samples. Isotopically-enriched compounds within generic Formula I can be prepared without undue experimentation by conventional techniques well known to those skilled in the art or by processes analogous to those described in the Schemes and Examples herein using appropriate isotopically-enriched reagents and/or intermediates.

It will be understood that, as used herein, references to the compounds of structural formula I are meant to also include the pharmaceutically acceptable salts, and also salts that are not pharmaceutically acceptable when they are used as precursors to the free compounds or their pharmaceutically acceptable salts or in other synthetic manipulations.

The compounds of the present invention may be administered in the form of a pharmaceutically acceptable salt. The term “pharmaceutically acceptable salt” refers to salts prepared from pharmaceutically acceptable non-toxic bases or acids including inorganic or organic bases and inorganic or organic acids. Salts of basic compounds encompassed within the term “pharmaceutically acceptable salt” refer to non-toxic salts of the compounds of this invention which are generally prepared by reacting the free base with a suitable organic or inorganic acid. Representative salts of basic compounds of the present invention include, but are not limited to, the following: acetate, benzenesulfonate, benzoate, bicarbonate, bisulfate, bitartrate, borate, bromide, camsylate, carbonate, chloride, clavulanate, citrate, edetate, edisylate, estolate, esylate, fumarate, gluceptate, gluconate, glutamate, hexylresorcinate, hydrobromide, hydrochloride, hydroxynaphthoate, iodide, isothionate, lactate, lactobionate, laurate, malate, maleate, mandelate, mesylate, methylbromide, methylnitrate, methylsulfate, mucate, napsylate, nitrate, N-methylglucamine ammonium salt, oleate, oxalate, pamoate (embonate), palmitate, pantothenate, phosphate/diphosphate, polygalacturonate, salicylate, stearate, sulfate, subacetate, succinate, tannate, tartrate, teoclate, tosylate, triethiodide and valerate. Furthermore, where the compounds of the invention carry an acidic moiety, suitable pharmaceutically acceptable salts thereof include, but are not limited to, salts derived from inorganic bases including aluminum, ammonium, calcium, copper, ferric, ferrous, lithium, magnesium, manganic, mangamous, potassium, sodium, zinc, and the like. Particularly preferred are the ammonium, calcium, magnesium, potassium, and sodium salts. Salts derived from pharmaceutically acceptable organic non-toxic bases include salts of primary, secondary, and tertiary amines, cyclic amines, and basic ion-exchange resins, such as arginine, betaine, caffeine, choline, N,N-dibenzylethylenediamine, diethylamine, 2-diethylaminoethanol, 2-dimethylaminoethanol, ethanolamine, ethylenediamine, N-ethylmorpholine, N-ethylpiperidine, glucamine, glucosamine, histidine, isopropylamine, lysine, methylglucamine, morpholine, piperazine, piperidine, polyamine resins, procaine, purines, theobromine, triethylamine, trimethylamine, tripropylamine, tromethamine, and the like.

Also, in the case of a carboxylic acid (—COOH) or alcohol group being present in the compounds of the present invention, pharmaceutically acceptable esters of carboxylic acid derivatives, such as methyl, ethyl, or pivaloyloxymethyl, or acyl derivatives of alcohols, such as acetyl, pivaloyl, benzoyl, and aminoacyl, can be employed. Included are those esters and acyl groups known in the art for modifying the solubility or hydrolysis characteristics for use as sustained-release or prodrug formulations.

Solvates, in particular hydrates, of the compounds of structural formula I are included in the present invention as well.

The subject compounds are useful in a method of inhibiting the stearoyl-coenzyme A delta-9 desaturase enzyme (SCD) in a patient such as a mammal in need of such inhibition comprising the administration of an effective amount of the compound. The compounds of the present invention are therefore useful to control, prevent, and/or treat conditions and diseases mediated by high or abnormal SCD enzyme activity.

Thus, one aspect of the present invention concerns a method of treating hyperglycemia, diabetes or insulin resistance in a mammalian patient in need of such treatment, which comprises administering to said patient an effective amount of a compound in accordance with structural formula I or a pharmaceutically salt or solvate thereof.

A second aspect of the present invention concerns a method of treating non-insulin dependent diabetes mellitus (Type 2 diabetes) in a mammalian patient in need of such treatment comprising administering to the patient an antidiabetic effective amount of a compound in accordance with structural formula I.

A third aspect of the present invention concerns a method of treating obesity in a mammalian patient in need of such treatment comprising administering to said patient a compound in accordance with structural formula I in an amount that is effective to treat obesity.

A fourth aspect of the invention concerns a method of treating metabolic syndrome and its sequelae in a mammalian patient in need of such treatment comprising administering to said patient a compound in accordance with structural formula I in an amount that is effective to treat metabolic syndrome and its sequelae. The sequelae of the metabolic syndrome include hypertension, elevated blood glucose levels, high triglycerides, and low levels of HDL cholesterol.

A fifth aspect of the invention concerns a method of treating a lipid disorder selected from the group conisting of dyslipidemia, hyperlipidemia, hypertriglyceridemia, hypercholesterolemia, low HDL and high LDL in a mammalian patient in need of such treatment comprising administering to said patient a compound in accordance with structural formula I in an amount that is effective to treat said lipid disorder.

A sixth aspect of the invention concerns a method of treating atherosclerosis in a mammalian patient in need of such treatment comprising administering to said patient a compound in accordance with structural formula I in an amount effective to treat atherosclerosis.

A seventh aspect of the invention concerns a method of treating cancer in a mammalian patient in need of such treatment comprising administering to said patient a compound in accordance with structural formula I in an amount effective to treat cancer. In one embodiment of this aspect of the invention, the cancer is liver cancer.

A further aspect of the invention concerns a method of treating a condition selected from the group consisting of (1) hyperglycemia, (2) low glucose tolerance, (3) insulin resistance, (4) obesity, (5) lipid disorders, (6) dyslipidemia, (7) hyperlipidemia, (8) hypertriglyceridemia, (9) hypercholesterolemia, (10) low HDL levels, (11) high LDL levels, (12) atherosclerosis and its sequelae, (13) vascular restenosis, (14) pancreatitis, (15) abdominal obesity, (16) neurodegenerative disease, (17) retinopathy, (18) nephropathy, (19) neuropathy, (20) non-alcoholic fatty liver disease or liver steatosis, (21) non-alcoholic steatohepatitis, (22) polycystic ovary syndrome, (23) sleep-disordered breathing, (24) metabolic syndrome, (25) liver fibrosis, (26) cirrhosis of the liver; and (27) other conditions and disorders where insulin resistance is a component, in a mammalian patient in need of such treatment comprising administering to the patient a compound in accordance with structural formula I in an amount that is effective to treat said condition.

Yet a further aspect of the invention concerns a method of delaying the onset of a condition selected from the group consisting of (1) hyperglycemia, (2) low glucose tolerance, (3) insulin resistance, (4) obesity, (5) lipid disorders, (6) dyslipidemia, (7) hyperlipidemia, (8) hypertriglyceridemia, (9) hypercholesterolemia, (10) low HDL levels, (11) high LDL levels, (12) atherosclerosis and its sequelae, (13) vascular restenosis, (14) pancreatitis, (15) abdominal obesity, (16) neurodegenerative disease, (17) retinopathy, (18) nephropathy, (19) neuropathy, (20) non-alcoholic fatty liver disease or liver steatosis, (21) non-alcoholic steatohepatitis, (22) polycystic ovary syndrome, (23) sleep-disordered breathing, (24) metabolic syndrome, (25) liver fibrosis, (26) cirrhosis of the liver; and (27) other conditions and disorders where insulin resistance is a component, in a mammalian patient in need of such treatment comprising administering to the patient a compound in accordance with structural formula I in an amount that is effective to delay the onset of said condition.

Yet a further aspect of the invention concerns a method of reducing the risk of developing a condition selected from the group consisting of (1) hyperglycemia, (2) low glucose tolerance, (3) insulin resistance, (4) obesity, (5) lipid disorders, (6) dyslipidemia, (7) hyperlipidemia, (8) hypertriglyceridemia, (9) hypercholesterolemia, (10) low HDL levels, (11) high LDL levels, (12) atherosclerosis and its sequelae, (13) vascular restenosis, (14) pancreatitis, (15) abdominal obesity, (16) neurodegenerative disease, (17) retinopathy, (18) nephropathy, (19) neuropathy, (20) non-alcoholic fatty liver disease or liver steatosis, (21) non-alcoholic steatohepatitis, (22) polycystic ovary syndrome, (23) sleep-disordered breathing, (24) metabolic syndrome, (25) liver fibrosis, (26) cirrhosis of the liver; and (27) other conditions and disorders where insulin resistance is a component, in a mammalian patient in need of such treatment comprising administering to the patient a compound in accordance with structural formula I in an amount that is effective to reduce the risk of developing said condition.

In addition to primates, such as humans, a variety of other mammals can be treated according to the method of the present invention. For instance, mammals including, but not limited to, cows, sheep, goats, horses, dogs, cats, guinea pigs, rats or other bovine, ovine, equine, canine, feline, rodent, such as a mouse, species can be treated. However, the method can also be practiced in other species, such as avian species (e.g., chickens).

The present invention is further directed to a method for the manufacture of a medicament for inhibiting stearoyl-coenzyme A delta-9 desaturase enzyme activity in humans and animals comprising combining a compound of the present invention with a pharmaceutically acceptable carrier or diluent. More particularly, the present invention is directed to the use of a compound of structural formula I in the manufacture of a medicament for use in treating a condition selected from the group consisting of hyperglycemia, Type 2 diabetes, insulin resistance, obesity, and a lipid disorder in a mammal, wherein the lipid disorder is selected from the group consisting of dyslipidemia, hyperlipidemia, hypertriglyceridemia, hypercholesterolemia, low HDL, and high LDL.

The subject treated in the present methods is generally a mammal, preferably a human being, male or female, in whom inhibition of stearoyl-coenzyme A delta-9 desaturase enzyme activity is desired. The term “therapeutically effective amount” means the amount of the subject compound that will elicit the biological or medical response of a tissue, system, animal or human that is being sought by the researcher, veterinarian, medical doctor or other clinician.

The term “composition” as used herein is intended to encompass a product comprising the specified ingredients in the specified amounts, as well as any product which results, directly or indirectly, from combination of the specified ingredients in the specified amounts. Such term in relation to pharmaceutical composition, is intended to encompass a product comprising the active ingredient(s) and the inert ingredient(s) that make up the carrier, as well as any product which results, directly or indirectly, from combination, complexation or aggregation of any two or more of the ingredients, or from dissociation of one or more of the ingredients, or from other types of reactions or interactions of one or more of the ingredients. Accordingly, the pharmaceutical compositions of the present invention encompass any composition made by admixing a compound of the present invention and a pharmaceutically acceptable carrier. By “pharmaceutically acceptable” it is meant the carrier, diluent or excipient must be compatible with the other ingredients of the formulation and not deleterious to the recipient thereof.

The terms “administration of” and or “administering a” compound should be understood to mean providing a compound of the invention or a prodrug of a compound of the invention to the individual in need of treatment.

The utility of the compounds in accordance with the present invention as inhibitors of stearoyl-coenzyme A delta-9 desaturase (SCD) enzyme activity may be demonstrated by the following microsomal and whole-cell based assays:

### I. SCD Enzyme Activity Assay:

The potency of compounds of formula I against the stearoyl-CoA desaturase was determined by measuring the conversion of radiolabeled stearoyl-CoA to oleoyl-CoA using rat liver microsome or human SCD1 (hSCD-1) following previously published procedures with some modifications (Joshi, et al., J. Lipid Res., 18: 32-36 (1977); Talamo, et al., Anal. Biochem, 29: 300-304 (1969)). Liver microsome was prepared from male Wistar or Sprague Dawley rats on a high carbohydrate diet for 3 days (LabDiet # 5803, Purina). The livers were homogenized (1:10 w/v) in a buffer containing 250 mM sucrose, 1 mM EDTA, 5 mM DTT and 50 mM Tris-HCl (pH 7.5). After a 100,000×g centrifugation for 60 min, the liver microsome pellet was suspended in a buffer containing 100 mM sodium phosphate, 20% glycerol, 2 mM DTT, and stored at −78° C. Human SCD1 desaturase system was reconstituted using human SCD1 from a baculovirus/Sf9 expression system, cytochrome B5 and cytochrome B5 reductase. Typically, test compound in 2 μL DMSO was incubated for 15 min at room temperature with 180 μL of the SCD enzyme in a buffer containing 100 mM Tris-HCl (pH 7.5), ATP (5 mM), Coenzyme-A (0.1 mM), Triton X-100 (0.5 mM) and NADH (2 mM). The reaction was initiated by the addition of 20 μL of [³H]-stearoyl-CoA (final concentration=2 μM, radioactivity concentration=1 μCi/mL). After 10 min, the reaction mixture (80 μL) was mixed with a calcium chloride/charcoal aqueous suspension (100 μl, charcoal (10% w/v) plus 25 μL CaCl₂ (2N). After centrifugation to precipitate the radioactive fatty acid species, tritiated water released from 9,10-[³H]-stearoyl-CoA by the SCD enzyme was quantified on a scintillation counter.

### II. Whole Cell-Based SCD (Delta-9), Delta-5 and Delta-6 Desaturase Assays:

Human HepG2 cells were grown on 96-well plates in MEM media (Gibco cat# 11095-072) supplemented with 10% heat-inactivated fetal bovine serum at 37° C. under 5% CO₂ in a humidified incubator. Test compound dissolved in the media was incubated with the sub-confluent cells for 15 min at 37° C. stearic acid was added to each well to a final concentration of 0.05 μCi/mL to detect SCD-catalyzed [¹⁴C]-oleic acid formation. 0.05 μCi/mL of [1-¹⁴C]-eicosatrienoic acid or [1-¹⁴C]-linolenic acid plus 10 μM of 2-amino-N-(3-chlorophenyl)benzamide (a delta-5 desaturase inhibitor) was used to index the delta-5 and delta-6 desaturase activities, respectively. After 4 h incubation at 37° C., the culture media was removed and the labeled cells were washed with PBS (3×1 mL) at room temperature. The labeled cellular lipids were hydrolyzed under nitrogen at 65° C. for 1 h using 400 μL of 2N sodium hydroxide plus 50 μL of L-α-phosphatidylcholine (2 mg/mL in isopropanol, Sigma #P-3556). After acidification with phosphoric acid (60 μL), the radioactive species were extracted with 300 μL of acetonitrile and quantified on a HPLC that was equipped with a C-18 reverse phase column and a Packard Flow Scintillation Analyzer. The levels of [¹⁴C]-oleic oleic acid over [¹⁴C]-stearic acid, [¹⁴C]-arachidonic acid over [¹⁴C]-eicosatrienoic acid, and [¹⁴C]-eicosatetraenoic acid (8,11,14,17) over [¹⁴C]-linolenic acid were used as the corresponding activity indices of SCD, delta-5 and delta-6 desaturase, respectively.

The SCD inhibitors of structural formula I, particularly the compounds of the present invention denoted as non-limiting specific Examples below, exhibit an inhibition constant IC₅₀ of less than 1 μM, and more typically less than 0.1 μM, against the rat and human SCD enzymes. Generally, the IC₅₀ ratio for delta-5 or delta-6 desaturases to human or rat SCD for a compound of structural formula I, particularly for the specific Examples denoted below, is at least about ten or more, and preferably about one hundred or greater.

### In Vivo Efficacy of Compounds of the Present Invention:

The in vivo efficacy of compounds of formula I can be determined by following the conversion of [1-¹⁴C]-stearic acid to [1-¹⁴C]oleic acid in animals as exemplified below. Mice are dosed with a compound of formula I and one hour later the radioactive tracer, [1-¹⁴C]-stearic acid, is dosed at 20 μCi/kg IV. At 3 h post dosing of the compound, the liver is harvested and then hydrolyzed in 10 N sodium hydroxide for 24 h at 80° C. After phosphoric acid acidification of the extract, the amount of stearic acid and [¹⁴C]-oleic acid is quantified on a HPLC system that was equipped with a C-18 reverse phase column and a Packard Flow Scintillation Analyzer

The subject compounds are further useful in a method for the prevention or treatment of the aforementioned diseases, disorders and conditions in combination with other agents.

The compounds of the present invention are further useful in methods for the prevention or treatment of the aforementioned diseases, disorders and conditions in combination with other therapeutic agents.

The compounds of the present invention may be used in combination with one or more other drugs in the treatment, prevention, suppression or amelioration of diseases or conditions for which compounds of Formula I or the other drugs may have utility, where the combination of the drugs together are safer or more effective than either drug alone. Such other drug(s) may be administered, by a route and in an amount commonly used therefor, contemporaneously or sequentially with a compound of Formula I. When a compound of Formula I is used contemporaneously with one or more other drugs, a pharmaceutical composition in unit dosage form containing such other drugs and the compound of Formula I is preferred, particularly in combination with a pharmaceutically acceptable carrier. However, the combination therapy may also include therapies in which the compound of Formula I and one or more other drugs are administered on different overlapping schedules. It is also contemplated that when used in combination with one or more other active ingredients, the compounds of the present invention and the other active ingredients may be used in lower doses than when each is used singly. Accordingly, the pharmaceutical compositions of the present invention include those that contain one or more other active ingredients, in addition to a compound of Formula I.

When a compound of the present invention is used contemporaneously with one or more other drugs, a pharmaceutical composition containing such other drugs in addition to the compound of the present invention is preferred. Accordingly, the pharmaceutical compositions of the present invention include those that also contain one or more other active ingredients, in addition to a compound of the present invention.

The weight ratio of the compound of the present invention to the second active ingredient may be varied and will depend upon the effective dose of each ingredient. Generally, an effective dose of each will be used. Thus, for example, when a compound of the present invention is combined with another agent, the weight ratio of the compound of the present invention to the other agent will generally range from about 1000:1 to about 1:1000, preferably about 200:1 to about 1:200. Combinations of a compound of the present invention and other active ingredients will generally also be within the aforementioned range, but in each case, an effective dose of each active ingredient should be used.

In such combinations the compound of the present invention and other active agents may be administered separately or in conjunction. In addition, the administration of one element may be prior to, concurrent to, or subsequent to the administration of other agent(s).

Examples of other active ingredients that may be administered in combination with a compound of Formula I, and either administered separately or in the same pharmaceutical composition, include, but are not limited to:

(1) dipeptidyl peptidase-IV (DPP-4) inhibitors;

(2) insulin sensitizers, including (i) PPARγ agonists, such as the glitazones (e.g. pioglitazone, rosiglitazone, netoglitazone, rivoglitazone, and balaglitazone) and other PPAR ligands, including (1) PPARα/γ, dual agonists, such as muraglitazar, aleglitazar, sodelglitazar, and naveglitazar, (2) PPARα agonists, such as fenofibric acid derivatives (gemfibrozil, clofibrate, ciprofibrate, fenofibrate and bezafibrate), (3) selective PPARγ modulators (SPPARγM's), such as those disclosed in WO 02/060388, WO 02/08188, WO 2004/019869, WO 2004/020409, WO 2004/020408, and WO 2004/066963, and (4) PPARγ partial agonists; (ii) biguanides, such as metformin and its pharmaceutically acceptable salts, in particular, metformin hydrochloride, and extended-release formulations thereof, such as Glumetza®, Fortamet®, and GlucophageXR®; (iii) protein tyrosine phosphatase-1B (PTP-1B) inhibitors;

(3) insulin and insulin analogs or derivatives, such as insulin lispro, insulin detemir, insulin glargine, insulin glulisine, and inhalable formulations of each thereof;

(4) leptin and leptin derivatives, agonists, and analogs, such as metreleptin;

(5) amylin; amylin analogs, such as davalintide; and amylin agonists, such as pramlintide;

(6) sulfonylurea and non-sulfonylurea insulin secretagogues, such as tolbutamide, glyburide, glipizide, glimepiride, mitiglinide, and meglitinides, such as nateglinide and repaglinide;

(7) α-glucosidase inhibitors (such as acarbose, voglibose and miglitol);

(8) glucagon receptor antagonists, such as those disclosed in WO 98/04528, WO 99/01423, WO 00/39088, and WO 00/69810;

(9) incretin mimetics, such as GLP-1, GLP-1 analogs, derivatives, and mimetics (See for example, WO 2008/011446, U.S. Pat. No. 5,545,618, U.S. Pat. No. 6,191,102, and U.S. Pat. No. 5,658,311); and GLP-1 receptor agonists, such as oxyntomodulin and its analogs and derivatives (See for example, WO 2003/022304, WO 2006/134340, WO 2007/100535), glucagon and its analogs and derivatives (See for example, WO 2008/101017), exenatide, liraglutide, taspoglutide, albiglutide, AVE0010, CJC-1134-PC, NN9535, LY2189265, LY2428757, and BIM-51077, including intranasal, transdermal, and once-weekly formulations thereof, such as exenatide QW;

(10) LDL cholesterol lowering agents such as (i) HMG-CoA reductase inhibitors (lovastatin, simvastatin, pravastatin, cerivastatin, fluvastatin, atorvastatin, pitavastatin, and rosuvastatin), (ii) bile acid sequestering agents (such as cholestyramine, colestimide, colesevelam hydrochloride, colestipol, and dialkylaminoalkyl derivatives of a cross-linked dextran, (iii) inhibitors of cholesterol absorption, such as ezetimibe, and (iv) acyl CoA:cholesterol acyltransferase inhibitors, such as avasimibe;

(11) HDL-raising drugs, such as niacin or a salt thereof and extended-release versions thereof; MK-524A, which is a combination of niacin extended-release and the DP-1 antagonist MK-524; and nicotinic acid receptor agonists;

(12) antiobesity compounds;

(13) agents intended for use in inflammatory conditions, such as aspirin, non-steroidal anti-inflammatory drugs (NSAIDs), glucocorticoids, and selective cyclooxygenase-2 (COX-2) inhibitors;

(14) antihypertensive agents, such as ACE inhibitors (such as enalapril, lisinopril, ramipril, captopril, quinapril, and tandolapril), A-II receptor blockers (such as losartan, candesartan, irbesartan, olmesartan medoxomil, valsartan, telmisartan, and eprosartan), renin inhibitors (such as aliskiren), beta blockers (such as and calcium channel blockers (such as;

(15) glucokinase activators (GKAs), such as LY2599506;

(16) inhibitors of 11β-hydroxysteroid dehydrogenase type 1, such as those disclosed in U.S. Pat. No. 6,730,690; WO 03/104207; and WO 04/058741;

(17) inhibitors of cholesteryl ester transfer protein (CETP), such as torcetrapib and MK-0859;

(18) inhibitors of fructose 1,6-bisphosphatase, such as those disclosed in U.S. Pat. Nos. 6,054,587; 6,110,903; 6,284,748; 6,399,782; and 6,489,476;

(19) inhibitors of acetyl CoA carboxylase-1 or 2 (ACC1 or ACC2);

(20) AMP-activated Protein Kinase (AMPK) activators;

(21) agonists of the G-protein-coupled receptors: GPR-109, GPR-116, GPR-119, and GPR-40;

(22) SSTR3 antagonists, such as those disclosed in WO 2009/011836;

(23) neuromedin U receptor 1 (NMUR1) and/or neuromedin U receptor 2 (NMUR2) agonists, such as those disclosed in WO2007/109135 and WO2009/042053, including, but not limited to, neuromedin U (NMU) and neuromedin S (NMS) and their analogs and derivatives;

(24) GPR-105 (P2YR14) antagonists, such as those disclosed in WO 2009/000087;

(25) inhibitors of glucose uptake, such as sodium-glucose transporter (SGLT) inhibitors and its various isoforms, such as SGLT-1; SGLT-2, such as dapagliflozin and remogliflozin; and SGLT-3;

(26) inhibitors of acyl coenzyme A:diacylglycerol acyltransferase 1 and 2 (DGAT-1 and DGAT-2);

(27) inhibitors of fatty acid synthase;

(28) inhibitors of acyl coenzyme A:monoacylglycerol acyltransferase 1 and 2 (MGAT-1 and MGAT-2);

(29) agonists of the TGR5 receptor (also known as GPBAR1, BG37, GPCR19, GPR131, and M-BAR);

(30) bromocriptine mesylate and rapid-release formulations thereof;

(31) histamine H3 receptor agonists; and

(32) α2-adrenergic or β3-adrenergic receptor agonists.

Dipeptidyl peptidase-IV (DPP-4) inhibitors that can be used in combination with compounds of Formula I include, but are not limited to, sitagliptin (disclosed in U.S. Pat. No. 6,699,871), vildagliptin, saxagliptin, alogliptin, denagliptin, carmegliptin, dutogliptin, melogliptin, linagliptin, and pharmaceutically acceptable salts thereof, and fixed-dose combinations of these compounds with metformin hydrochloride, pioglitazone, rosiglitazone, simvastatin, atorvastatin, or a sulfonylurea.

Other dipeptidyl peptidase-IV (DPP-4) inhibitors that can be used in combination with compounds of Formula I include, but are not limited to:
(2R,3S,5R)-5-(1-methyl-4,6-dihydropyrrolo[3,4-c]pyrazol-5(1H)-yl)-2-(2,4,5-trifluorophenyl)tetrahydro-2H-pyran-3-amine;
(2R,3S,5R)-5-(1-methyl-4,6-dihydropyrrolo[3,4-c]pyrazol-5(1H)-yl)-2-(2,4,5-trifluorophenyl)tetrahydro-2H-pyran-3-amine;
(2R,3S,5R)-2-(2,5-difluorophenyl)tetrahydro)-5-(4,6-dihydropyrrolo[3,4-c]pyrazol-5(1H)-yl)tetrahydro-2H-pyran-3-amine;
(3R)-4-[(3R)-3-amino-4-(2,4,5-trifluorophenyl)butanoyl]-hexahydro-3-methyl-2H-1,4-diazepin-2-one;
4-[(3R)-3-amino-4-(2,5-difluorophenyl)butanoyl]hexahydro-1-methyl-2H-1,4-diazepin-2-one hydrochloride; and
(3R)-4-[(3R)-3-amino-4-(2,4,5-trifluorophenyl)butanoyl]-hexahydro-3-(2,2,2-trifluoroethyl)-2H-1,4-diazepin-2-one; and
   pharmaceutically acceptable salts thereof.

Antiobesity compounds that can be combined with compounds of Formula I include topiramate; zonisamide; naltrexone; phentermine; bupropion; the combination of bupropion and naltrexone; the combination of bupropion and zonisamide; the combination of topiramate and phentermine; fenfluramine; dexfenfluramine; sibutramine; lipase inhibitors, such as orlistat and cetilistat; melanocortin receptor agonists, in particular, melanocortin-4 receptor agonists; CCK-1 agonists; melanin-concentrating hormone (MCH) receptor antagonists; neuropeptide Y₁ or Y₅ antagonists (such as MK-0557); CB1 receptor inverse agonists and antagonists (such as rimonabant and taranabant); β₃ adrenergic receptor agonists; ghrelin antagonists; bombesin receptor agonists (such as bombesin receptor subtype-3 agonists); histamine H3 receptor inverse agonists; 5-hydroxytryptamine-2c (5-HT2c) agonists, such as lorcaserin; and inhibitors of fatty acid synthase (FAS). For a review of anti-obesity compounds that can be combined with compounds of the present invention, see S. Chaki et al., “Recent advances in feeding suppressing agents: potential therapeutic strategy for the treatment of obesity,” Expert Opin. Ther. Patents, 11: 1677-1692 (2001); D. Spanswick and K. Lee, “Emerging antiobesity drugs,” Expert Opin. Emerging Drugs, 8: 217-237 (2003); J. A. Fernandez-Lopez, et al., “Pharmacological Approaches for the Treatment of Obesity,” Drugs, 62: 915-944 (2002); and K. M. Gadde, et al., “Combination pharmaceutical therapies for obesity,” Exp. Opin. Pharmacother., 10: 921-925 (2009).

Glucagon receptor antagonists that can be used in combination with the compounds of Formula I include, but are not limited to:
N-[4-((1S)-1-{3-(3,5-dichlorophenyl)-5-[6-(trifluoromethoxy)-2-naphthyl]-1H-pyrazol-1-yl}ethyl)benzoyl]-β-alanine;
N-[4-((1R)-1-{3-(3,5-dichlorophenyl)-5-[6-(trifluoromethoxy)-2-naphthyl]-1H-pyrazol-1-yl}ethyl)benzoyl]-O-alanine;
N-(4-{1-[3-(2,5-dichlorophenyl)-5-(6-methoxy-2-naphthyl)-1H-pyrazol-1-yl]ethyl}benzoyl)-β-alanine;
N-(4-{(1S)-1-[3-(3,5-dichlorophenyl)-5-(6-methoxy-2-naphthyl)-1H-pyrazol-1-yl]ethyl}benzoyl)-β-alanine;
N-(4-{(1S)-1-[(R)-(4-chlorophenyl)(7-fluoro-5-methyl-1H-indol-3-yl)methyl]butyl}benzoyl)-β-alanine; and
N-(4-{(1S)-1-[(4-chlorophenyl)(6-chloro-8-methylquinolin-4-yl)methyl]butyl}benzoyl)-β-alanine; and
   pharmaceutically acceptable salts thereof.

Agonists of the GPR-119 receptor that can be used in combination with the compounds of Formula I include, but are not limited to: rac-cis 5-chloro-2-{4-[2-(2-{[5-(methylsulfonyl)pyridin-2-yl]oxy}ethyl)cyclopropyl]piperidin-1-yl}pyrimidine;
5-chloro-2-{4-[(1R,2S)-2-(2-{[5-(methylsulfonyl)pyridin-2-yl]oxy}ethyl)cyclopropyl]piperidin-1-yl}pyrimidine;
rac cis-5-chloro-2-[4-(2-{2-[4-(methylsulfonyl)phenoxy]ethyl}cyclopropyl)piperidin-1-yl]pyrimidine;
5-chloro-2-[4-((1S,2R)-2-{2-[4-(methylsulfonyl)phenoxy]ethyl}cyclopropyl)piperidin-1-yl]pyrimidine;
5-chloro-2-[4-((1R,2S)-2-{2-[4-(methylsulfonyl)phenoxy]ethyl}cyclopropyl)piperidin-1-yl]pyrimidine;
rac cis-5-chloro-2-[4-(2-{2-[3-(methylsulfonyl)phenoxy]ethyl}cyclopropyl)piperidin-1-yl]pyrimidine; and
rac cis-5-chloro-2-[4-(2-{2-[3-(5-methyl-1,3,4-oxadiazol-2-yl)phenoxy]ethyl}cyclopropyl)piperidin-1-yl]pyrimidine; and
   pharmaceutically acceptable salts thereof.

Selective PPARγ modulators (SPPARγM's) that can be used in combination with the compounds of Formula I include, but are not limited to:
(2S)-2-({6-chloro-3-[6-(4-chlorophenoxy)-2-propylpyridin-3-yl]-1,2-benzisoxazol-5-yl}oxy)propanoic acid;
(2S)-2-({6-chloro-3-[6-(4-fluorophenoxy)-2-propylpyridin-3-yl]-1,2-benzisoxazol-5-yl}oxy)propanoic acid;
(2S)-2-{[6-chloro-3-(6-phenoxy-2-propylpyridin-3-yl)-1,2-benzisoxazol-5-yl]oxy}propanoic acid;
(2R)-2-({6-chloro-3-[6-(4-chlorophenoxy)-2-propylpyridin-3-yl]-1,2-benzisoxazol-5-yl}oxy)propanoic acid;
(2R)-2-{3-[3-(4-methoxy)benzoyl-2-methyl-6-(trifluoromethoxy)-1H-indol-1-yl]phenoxy}butanoic acid;
(2S)-2-{3-[3-(4-methoxy)benzoyl-2-methyl-6-(trifluoromethoxy)-1H-indol-1-yl]phenoxy}butanoic acid;
2-{3-[3-(4-methoxy)benzoyl-2-methyl-6-(trifluoromethoxy)-1H-indol-1-yl]phenoxy}-2-methylpropanoic acid; and
(2R)-2-{3-[3-(4-chloro)benzoyl-2-methyl-6-(trifluoromethoxy)-1H-indol-1-yl]phenoxy}propanoic acid; and
   pharmaceutically acceptable salts thereof.

Inhibitors of 11β-hydroxysteroid dehydrogenase type 1 that can be used in combination with the compounds of Formula I include, but are not limited to:
3-[1-(4-chlorophenyl)-trans-3-fluorocyclobutyl]-4,5-dicyclopropyl-r-4H-1,2,4-triazole;
3-[1-(4-chlorophenyl)-trans-3-fluorocyclobutyl]-4-cyclopropyl-5-(1-methylcyclopropyl)-r-4H-1,2,4-triazole;
3-[1-(4-chlorophenyl)-trans-3-fluorocyclobutyl]-4-methyl-5-[2-(trifluoromethoxy)phenyl]-r-4H-1,2,4-triazole;
3-[1-(4-chlorophenyl)cyclobutyl]-4-methyl-5-[2-(trifluoromethyl)phenyl]-4H-1,2,4-triazole;
3-{4-[3-(ethylsulfonyl)propyl]bicyclo[2.2.2]oct-1-yl}-4-methyl-5-[2-(trifluoromethyl)phenyl]-4H-1,2,4-triazole;
4-methyl-3-{4-[4-(methylsulfonyl)phenyl]bicyclo[2.2.2]oct-1-yl}-5-[2-(trifluoromethyl)phenyl]-4H-1,2,4-triazole;
3-(4-{4-methyl-5-[2-(trifluoromethyl)phenyl]-4H-1,2,4-triazol-3-yl}bicyclo[2.2.2]oct-1-yl)-5-(3,3,3-trifluoropropyl)-1,2,4-oxadiazole;
3-(4-{4-methyl-5-[2-(trifluoromethyl)phenyl]-4H-1,2,4-triazol-3-yl}bicyclo[2.2.2]oct-1-yl)-5-(3,3,3-trifluoroethyl)-1,2,4-oxadiazole;
5-(3,3-difluorocyclobutyl)-3-(4-{4-methyl-5-[2-(trifluoromethyl)phenyl]-4H-1,2,4-triazol-3-yl}bicyclo[2.2.2]oct-1-yl)-1,2,4-oxadiazole;
5-(1-fluoro-1-methylethyl)-3-(4-{4-methyl-5-[2-(trifluoromethyl)phenyl]-4H-1,2,4-triazol-3-yl}bicyclo[2.2.2]oct-1-yl)-1,2,4-oxadiazole;
2-(1,1-difluoroethyl)-5-(4-{4-methyl-5-[2-(trifluoromethyl)phenyl]-4H-1,2,4-triazol-3-yl}bicyclo[2.2.2]oct-1-yl)-1,3,4-oxadiazole;
2-(3,3-difluorocyclobutyl)-5-(4-{4-methyl-5-[2-(trifluoromethyl)phenyl]-4H-1,2,4-triazol-3-yl}bicyclo[2.2.2]oct-1-yl)-1,3,4-oxadiazole; and
5-(1,1-difluoroethyl)-3-(4-{4-methyl-5-[2-(trifluoromethyl)phenyl]-4H-1,2,4-triazol-3-yl}bicyclo[2.2.2]oct-1-yl)-1,2,4-oxadiazole; and
   pharmaceutically acceptable salts thereof.

Somatostatin subtype receptor 3 (SSTR3) antagonists that can be used in combination with the compounds of Formula I include, but are not limited to:

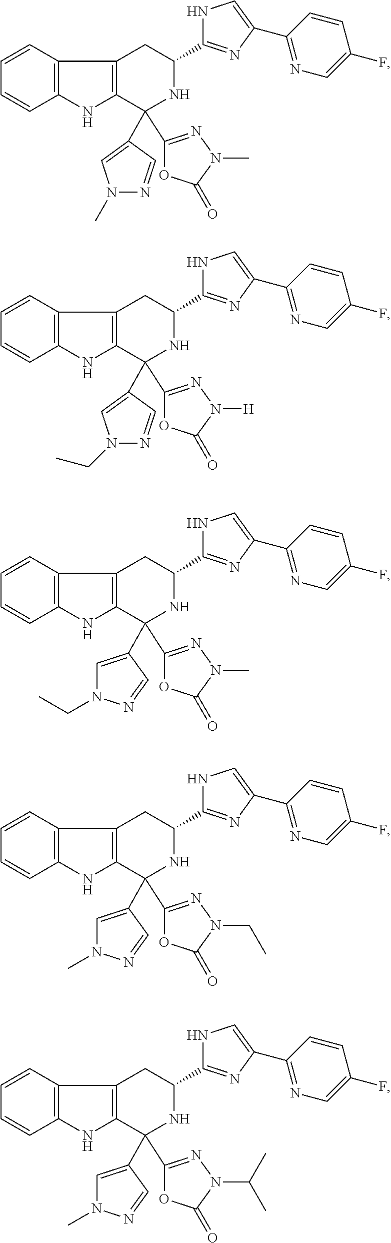

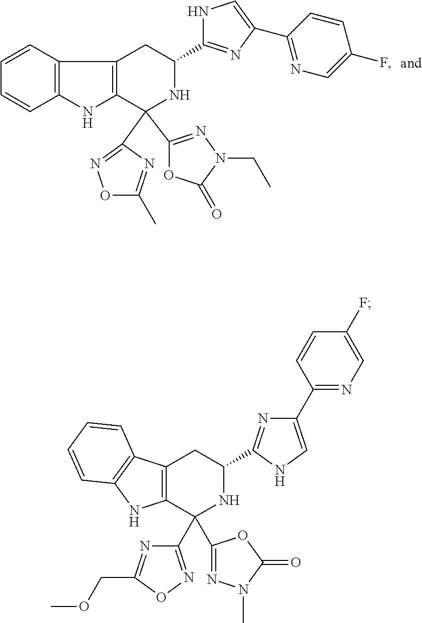

and pharmaceutically acceptable salts thereof.

AMP-activated Protein Kinase (AMPK) activators that can be used in combination with the compounds of Formula I include, but are not limited to:

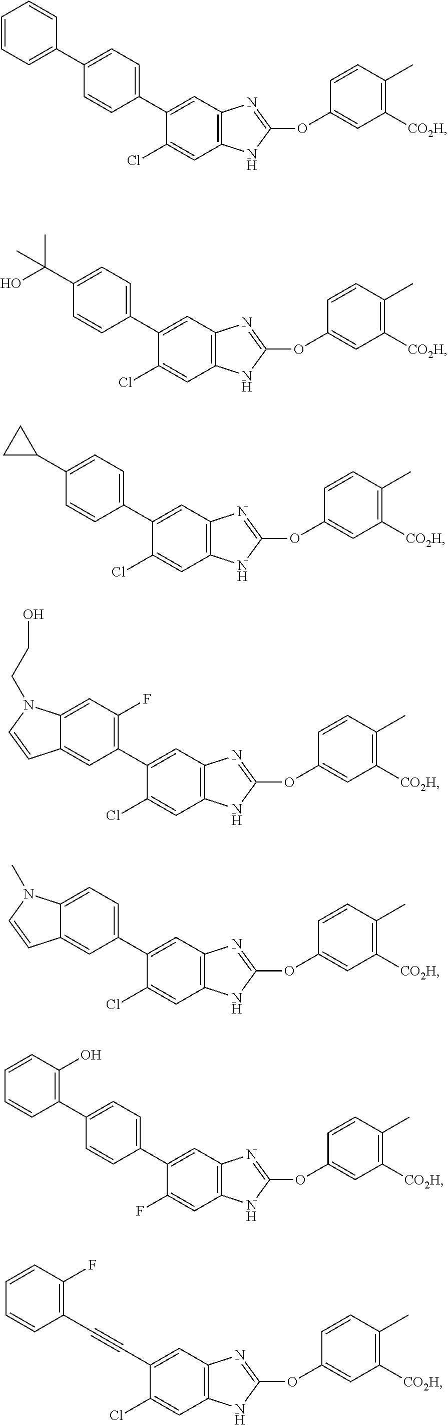

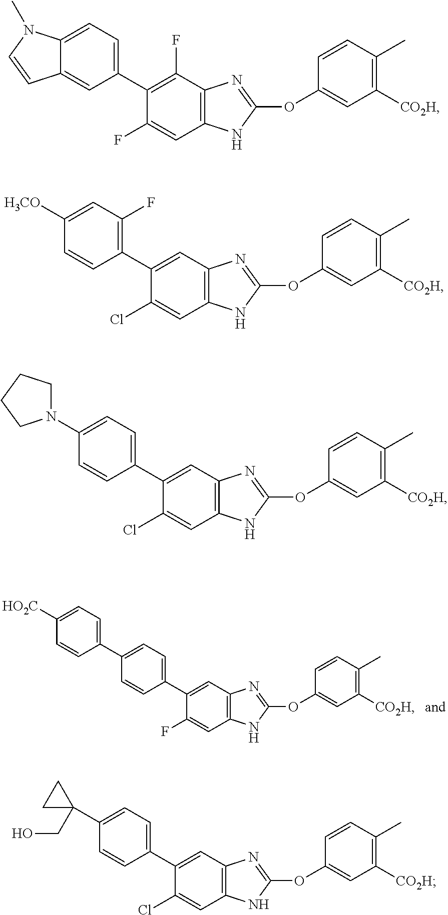

and pharmaceutically acceptable salts thereof.

Inhibitors of acetyl-CoA carboxylase-1 and 2 (ACC-1 and ACC-2) that can be used in combination with the compounds of Formula I include, but are not limited to:
3-{1′-[(1-cyclopropyl-4-methoxy-1H-indol-6-yl)carbonyl]-4-oxospiro[chroman-2,4′-piperidin]-6-yl}benzoic acid;
5-{1′-[(1-cyclopropyl-4-methoxy-1H-indol-6-yl)carbonyl]-4-oxospiro[chroman-2,4′-piperidin]-6-yl}nicotinic acid;
1′-[(1-cyclopropyl-4-methoxy-1H-indol-6-yl)carbonyl]-6-(1H-tetrazol-5-yl)spiro[chroman-2,4′-piperidin]-4-one;
1′-[(1-cyclopropyl-4-ethoxy-3-methyl-1H-indol-6-yl)carbonyl]-6-(1H-tetrazol-5-yl)spiro[chroman-2,4′-piperidin]-4-one;
5-{1′-[(1-cyclopropyl-4-methoxy-3-methyl-1H-indol-6-yl)carbonyl]-4-oxo-spiro[chroman-2,4′-piperidin]-6-yl}nicotinic acid;
4′-({6-(5-carbamoylpyridin-2-yl)-4-oxospiro[chroman-2,4′-piperidin]-1′-yl}carbonyl)-2′,6′-diethoxybiphenyl-4-carboxylic acid;
2′,6′-diethoxy-4′-{[6-(1-methyl-1H-pyrazol-4-yl)-4-oxospiro[chroman-2,4′-piperidin]-1′-yl]carbonyl}biphenyl-4-carboxylic acid;
2′,6′-diethoxy-3-fluoro-4′-{[6-(1-methyl-1H-pyrazol-4-yl)-4-oxospiro[chroman-2,4′-piperidin]-1′-yl]carbonyl}biphenyl-4-carboxylic acid;
5-[4-({6-(3-carbamoylphenyl)-4-oxospiro[chroman-2,4′-piperidin]-1′-yl}carbonyl)-2,6-diethoxyphenyl]nicotinic acid;
sodium 4′-({6-(5-carbamoylpyridin-2-yl)-4-oxospiro[chroman-2,4′-piperidin]-1′-yl}carbonyl)-2′,6′-diethoxybiphenyl-4-carboxylate;
methyl 4′-({6-(5-carbamoylpyridin-2-yl)-4-oxospiro[chroman-2,4′-piperidin]-1′-yl}carbonyl)-2′,6′-diethoxybiphenyl-4-carboxylate;
1′-[(4,8-dimethoxyquinolin-2-yl)carbonyl]-6-(1H-tetrazol-5-yOspiro[chroman-2,4′-piperidin]-4-one;
(5-{1′-[(4,8-dimethoxyquinolin-2-yl)carbonyl]-4-oxospiro[chroman-2,4′-piperidin]-6-yl}-2H-tetrazol-2-yl)methyl pivalate;
5-{1′-[(8-cyclopropyl-4-methoxyquinolin-2-yl)carbonyl]-4-oxospiro[chroman-2,4′-piperidin]-6-yl}nicotinic acid;
1′-(8-methoxy-4-morpholin-4-yl-2-naphthoyl)-6-(1H-tetrazol-5-yl)spiro[chroman-2,4′-piperidin]-4-one; and
1′-[(4-ethoxy-8-ethylquinolin-2-yl)carbonyl]-6-(1H-tetrazol-5-yl)spiro[chroman-2,4′-piperidin]-4-one; and
   pharmaceutically acceptable salts and esters thereof.

One particular aspect of combination therapy concerns a method of treating a condition selected from the group consisting of hypercholesterolemia, atherosclerosis, low HDL levels, high LDL levels, hyperlipidemia, hypertriglyceridemia, and dyslipidemia, in a mammalian patient in need of such treatment comprising administering to the patient a therapeutically effective amount of a compound of structural formula I and an HMG-CoA reductase inhibitor.

More particularly, this aspect of combination therapy concerns a method of treating a condition selected from the group consisting of hypercholesterolemia, atherosclerosis, low HDL levels, high LDL levels, hyperlipidemia, hypertriglyceridemia and dyslipidemia in a mammalian patient in need of such treatment wherein the HMG-CoA reductase inhibitor is a statin selected from the group consisting of lovastatin, simvastatin, pravastatin, cerivastatin, fluvastatin, atorvastatin, and rosuvastatin.

In another aspect of the invention, a method of reducing the risk of developing a condition selected from the group consisting of hypercholesterolemia, atherosclerosis, low HDL levels, high LDL levels, hyperlipidemia, hypertriglyceridemia and dyslipidemia, and the sequelae of such conditions is disclosed comprising administering to a mammalian patient in need of such treatment a therapeutically effective amount of a compound of structural formula I and an HMG-CoA reductase inhibitor.

In another aspect of the invention, a method for delaying the onset or reducing the risk of developing atherosclerosis in a human patient in need of such treatment is disclosed comprising administering to said patient an effective amount of a compound of structural formula I and an HMG-CoA reductase inhibitor.

More particularly, a method for delaying the onset or reducing the risk of developing atherosclerosis in a human patient in need of such treatment is disclosed, wherein the HMG-CoA reductase inhibitor is a statin selected from the group consisting of: lovastatin, simvastatin, pravastatin, cerivastatin, fluvastatin, atorvastatin, and rosuvastatin.

In another aspect of the invention, a method for delaying the onset or reducing the risk of developing atherosclerosis in a human patient in need of such treatment is disclosed, wherein the HMG-Co A reductase inhibitor is a statin and further comprising administering a cholesterol absorption inhibitor.

More particularly, in another aspect of the invention, a method for delaying the onset or reducing the risk of developing atherosclerosis in a human patient in need of such treatment is disclosed, wherein the HMG-Co A reductase inhibitor is a statin and the cholesterol absorption inhibitor is ezetimibe.

The compounds of the present invention may be administered by oral, parenteral (e.g., intramuscular, intraperitoneal, intravenous, ICV, intracisternal injection or infusion, subcutaneous injection, or implant), by inhalation spray, nasal, vaginal, rectal, sublingual, or topical routes of administration and may be formulated, alone or together, in suitable dosage unit formulations containing conventional non-toxic pharmaceutically acceptable carriers, adjuvants and vehicles appropriate for each route of administration. In addition to the treatment of warm-blooded animals such as mice, rats, horses, cattle, sheep, dogs, cats, monkeys, etc., the compounds of the invention are effective for use in humans.

The pharmaceutical compositions for the administration of the compounds of this invention may conveniently be presented in dosage unit form and may be prepared by any of the methods well known in the art of pharmacy. All methods include the step of bringing the active ingredient into association with the carrier which constitutes one or more accessory ingredients. In general, the pharmaceutical compositions are prepared by uniformLy and intimately bringing the active ingredient into association with a liquid carrier or a finely divided solid carrier or both, and then, if necessary, shaping the product into the desired formulation. In the pharmaceutical composition the active object compound is included in an amount sufficient to produce the desired effect upon the process or condition of diseases. As used herein, the term “composition” is intended to encompass a product comprising the specified ingredients in the specified amounts, as well as any product which results, directly or indirectly, from combination of the specified ingredients in the specified amounts.

The pharmaceutical compositions containing the active ingredient may be in a form suitable for oral use, for example, as tablets, troches, lozenges, aqueous or oily suspensions, dispersible powders or granules, emulsions, hard or soft capsules, or syrups or elixirs. Compositions intended for oral use may be prepared according to any method known to the art for the manufacture of pharmaceutical compositions and such compositions may contain one or more agents selected from the group consisting of sweetening agents, flavoring agents, coloring agents and preserving agents in order to provide pharmaceutically elegant and palatable preparations. Tablets contain the active ingredient in admixture with non-toxic pharmaceutically acceptable excipients which are suitable for the manufacture of tablets. These excipients may be for example, inert diluents, such as calcium carbonate, sodium carbonate, lactose, calcium phosphate or sodium phosphate; granulating and disintegrating agents, for example, corn starch, or alginic acid; binding agents, for example starch, gelatin or acacia, and lubricating agents, for example magnesium stearate, stearic acid or talc. The tablets may be uncoated or they may be coated by known techniques to delay disintegration and absorption in the gastrointestinal tract and thereby provide a sustained action over a longer period. For example, a time delay material such as glyceryl monostearate or glyceryl distearate may be employed. They may also be coated by the techniques described in the U.S. Pat. Nos. 4,256,108; 4,166,452; and 4,265,874 to form osmotic therapeutic tablets for control release.

Formulations for oral use may also be presented as hard gelatin capsules wherein the active ingredient is mixed with an inert solid diluent, for example, calcium carbonate, calcium phosphate or kaolin, or as soft gelatin capsules wherein the active ingredient is mixed with water or an oil medium, for example peanut oil, liquid paraffin, or olive oil.

Aqueous suspensions contain the active materials in admixture with excipients suitable for the manufacture of aqueous suspensions. Such excipients are suspending agents, for example sodium carboxymethylcellulose, methylcellulose, hydroxypropylmethylcellulose, sodium alginate, polyvinyl-pyrrolidone, gum tragacanth and gum acacia; dispersing or wetting agents may be a naturally-occurring phosphatide, for example lecithin, or condensation products of an alkylene oxide with fatty acids, for example polyoxyethylene stearate, or condensation products of ethylene oxide with long chain aliphatic alcohols, for example heptadecaethyleneoxycetanol, or condensation products of ethylene oxide with partial esters derived from fatty acids and a hexitol such as polyoxyethylene sorbitol monooleate, or condensation products of ethylene oxide with partial esters derived from fatty acids and hexitol anhydrides, for example polyethylene sorbitan monooleate. The aqueous suspensions may also contain one or more preservatives, for example ethyl or n-propyl p-hydroxybenzoate, one or more coloring agents, one or more flavoring agents, and one or more sweetening agents, such as sucrose or saccharin.

Oily suspensions may be formulated by suspending the active ingredient in a vegetable oil, for example arachis oil, olive oil, sesame oil or coconut oil, or in a mineral oil such as liquid paraffin. The oily suspensions may contain a thickening agent, for example beeswax, hard paraffin or cetyl alcohol. Sweetening agents such as those set forth above, and flavoring agents may be added to provide a palatable oral preparation. These compositions may be preserved by the addition of an anti-oxidant such as ascorbic acid.

Dispersible powders and granules suitable for preparation of an aqueous suspension by the addition of water provide the active ingredient in admixture with a dispersing or wetting agent, suspending agent and one or more preservatives. Suitable dispersing or wetting agents and suspending agents are exemplified by those already mentioned above. Additional excipients, for example sweetening, flavoring and coloring agents, may also be present.

The pharmaceutical compositions of the invention may also be in the form of oil-in-water emulsions. The oily phase may be a vegetable oil, for example olive oil or arachis oil, or a mineral oil, for example liquid paraffin or mixtures of these. Suitable emulsifying agents may be naturally—occurring gums, for example gum acacia or gum tragacanth, naturally-occurring phosphatides, for example soy bean, lecithin, and esters or partial esters derived from fatty acids and hexitol anhydrides, for example sorbitan monooleate, and condensation products of the said partial esters with ethylene oxide, for example polyoxyethylene sorbitan monooleate. The emulsions may also contain sweetening and flavoring agents.

Syrups and elixirs may be formulated with sweetening agents, for example glycerol, propylene glycol, sorbitol or sucrose. Such formulations may also contain a demulcent, a preservative and flavoring and coloring agents.

The pharmaceutical compositions may be in the form of a sterile injectable aqueous or oleagenous suspension. This suspension may be formulated according to the known art using those suitable dispersing or wetting agents and suspending agents which have been mentioned above. The sterile injectable preparation may also be a sterile injectable solution or suspension in a non-toxic parenterally-acceptable diluent or solvent, for example as a solution in 1,3-butanediol. Among the acceptable vehicles and solvents that may be employed are water, Ringer's solution and isotonic sodium chloride solution. In addition, sterile, fixed oils are conventionally employed as a solvent or suspending medium. For this purpose any bland fixed oil may be employed including synthetic mono- or diglycerides. In addition, fatty acids such as oleic acid find use in the preparation of injectables.

The compounds of the present invention may also be administered in the form of suppositories for rectal administration of the drug. These compositions can be prepared by mixing the drug with a suitable non-irritating excipient which is solid at ordinary temperatures but liquid at the rectal temperature and will therefore melt in the rectum to release the drug. Such materials are cocoa butter and polyethylene glycols.

For topical use, creams, ointments, jellies, solutions or suspensions, etc., containing the compounds of the present invention are employed. (For purposes of this application, topical application shall include mouthwashes and gargles.)

The pharmaceutical composition and method of the present invention may further comprise other therapeutically active compounds as noted herein which are usually applied in the treatment of the above mentioned pathological conditions.

In the treatment or prevention of conditions which require inhibition of stearoyl-CoA delta-9 desaturase enzyme activity an appropriate dosage level will generally be about 0.01 to 500 mg per kg patient body weight per day which can be administered in single or multiple doses. Preferably, the dosage level will be about 0.1 to about 250 mg/kg per day; more preferably about 0.5 to about 100 mg/kg per day. A suitable dosage level may be about 0.01 to 250 mg/kg per day, about 0.05 to 100 mg/kg per day, or about 0.1 to 50 mg/kg per day. Within this range the dosage may be 0.05 to 0.5, 0.5 to 5 or 5 to 50 mg/kg per day. For oral administration, the compositions are preferably provided in the form of tablets containing 1.0 to 1000 mg of the active ingredient, particularly 1.0, 5.0, 10.0, 15.0. 20.0, 25.0, 50.0, 75.0, 100.0, 150.0, 200.0, 250.0, 300.0, 400.0, 500.0, 600.0, 750.0, 800.0, 900.0, and 1000.0 mg of the active ingredient for the symptomatic adjustment of the dosage to the patient to be treated. The compounds may be administered on a regimen of 1 to 4 times per day, preferably once or twice per day.

When treating or preventing diabetes mellitus and/or hyperglycemia or hypertriglyceridemia or other diseases for which compounds of the present invention are indicated, generally satisfactory results are obtained when the compounds of the present invention are administered at a daily dosage of from about 0.1 mg to about 100 mg per kilogram of animal body weight, preferably given as a single daily dose or in divided doses two to six times a day, or in sustained release form. For most large mammals, the total daily dosage is from about 1.0 mg to about 1000 mg, preferably from about 1 mg to about 50 mg. In the case of a 70 kg adult human, the total daily dose will generally be from about 7 mg to about 350 mg. This dosage regimen may be adjusted to provide the optimal therapeutic response.

It will be understood, however, that the specific dose level and frequency of dosage for any particular patient may be varied and will depend upon a variety of factors including the activity of the specific compound employed, the metabolic stability and length of action of that compound, the age, body weight, general health, sex, diet, mode and time of administration, rate of excretion, drug combination, the severity of the particular condition, and the host undergoing therapy.

### Preparation of Compounds of the Invention:

Synthetic methods for preparing the compounds of the present invention are illustrated in the following Schemes, Methods, and Examples. Starting materials are commercially available or may be made according to procedures known in the art or as illustrated herein. The compounds of the invention are illustrated by means of the specific examples shown below. However, these specific examples are not to be construed as forming the only genus that is considered as the invention. These examples further illustrate details for the preparation of the compounds of the present invention. Those skilled in the art will readily understand that known variations of the conditions and processes of the following preparative procedures can be used to prepare these compounds. All temperatures are degrees Celsius unless otherwise noted. Mass spectra (MS) were measured by electrospray ion-mass spectroscopy (ESI). ¹H NMR spectra were recorded on Bruker instruments at 400 or 500 MHz.

### LIST OF ABBREVIATIONS

Alk=alkyl
Aq=aqueous
Ar=aryl
BINAP=2,2′-bis(diphenylphosphino)-1,1′-binaphthalene
Boc=tert-butoxycarbonyl
br=broad
n-BuLi=n-butyllithium
t-BuLi=tert-butyllithium
CAN=ceric ammonium nitrate
CH₂Cl₂=dichloromethane
d=doublet
DAST=Diethylaminosulfur trifluoride
dd=doublet of doublet
DBU=1,8-diazabicyclo[5.4.0]undec-7-ene
DDQ=2,3-dichloro-5,6-dicyano-1,4-benzoquinone
DEAD=diethyl azodicarboxylate
DIPEA=N,N-diisopropylethylamine
DMAP=4-dimethylaminopyridine
DMF=N,N-dimethylformamide
DMSO=dimethyl sulfoxide
ESI=electrospray ionization
Et=ethyl
Et₂O=diethyl ether
Et₃N=triethylamine
EtOAc=ethyl acetate
EtOH=ethyl alcohol
HATU=O-(7-azabenzotriazol-1-yl)-N,N,N,N′-tetramethyluronium hexafluorophosphate
HOAc=acetic acid
LDA=lithium diisopropylamide
LiOH=lithium hydroxide
m=multiplet
Me=methyl
MeCN=acetonitrile
MeOH=methyl alcohol
MeTHF=2-methyltetrahydrofuran
min=minutes
MgSO₄=magnesium sulfate
MS=mass spectroscopy
MTBE=methyl tert-butyl ether
N=normal
NaOH=sodium hydroxide
Na₂SO₄=sodium sulfate
NBS=N-bromosuccinimide
NMP=N-methyl 2-pyrrolidinone
NMR=nuclear magnetic resonance spectroscopy
PG=protecting group
Ph=phenyl
rt=room temperature
s=singlet
sat.=saturated
t=triplet
td=triplet of doublet
TFAA=trifluoroacetic anhydride
THF=tetrahydrofuran
TMEDA=N,N,N′,N′-tetramethylethylenediamine

### Method A:

An appropriately substituted heteroaryl bromide 1 is reacted with concentrated ammonium hydroxide in a solvent such as THF to give amide 2. Dehydration with TFAA in a solvent like CH₂Cl₂ gives the nitrile intermediate 3. The nitrile intermediate 3 is reacted with NaN₃ in the presence of a Lewis acid catalyst such as ZnBr₂ and a solvent such as 2-propanol. The tetrazole 4 is then reacted with ethyl bromoacetate in the presence of a base such as Et₃N or an alkali metal (K, Na, Cs) carbonate in a solvent such as THF, 1,4-dioxane or DMF at a temperature range of room temperature to refluxing temperature. The 2-alkylated ester tetrazole 5 is typically obtained together with the 1-alkylated isomer 6 which can be separated by standard chromatographic methods.

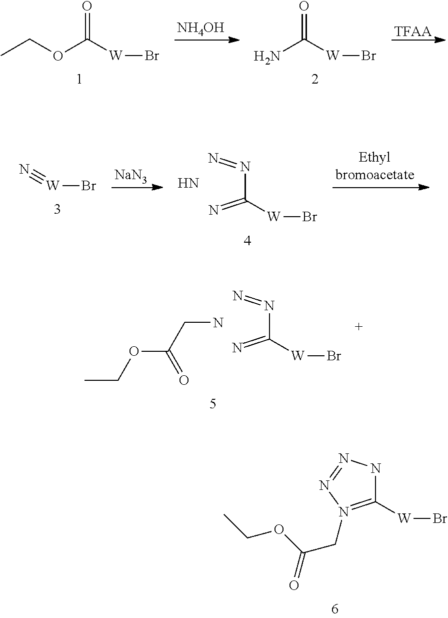

### Method B:

Alternatively, the tetrazole intermediate 4 can be reacted with t-butyl bromoacetate in the presence of a base such as Et₃N or an alkali metal (K, Na, Cs) carbonate in a solvent such as THF, 1,4-dioxane or DMF at a temperature range of room temperature to refluxing temperature. The 2-alkylated ester tetrazole 7 is typically obtained together with the 1-alkylated isomer 8 which can be separated by standard chromatographic methods.

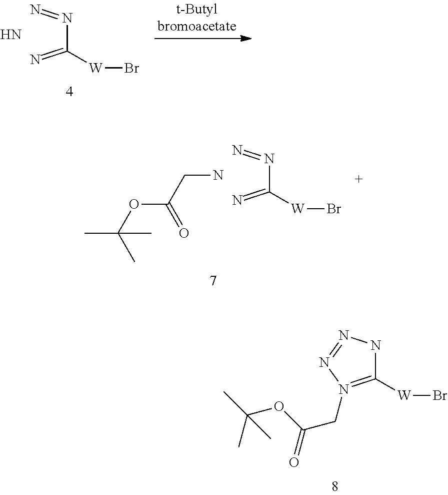

### Method C:

Where W represents an isoxazole ring, a mixture of the oxime 9 and an acrylate 10 is reacted at a temperature range of −78° C. to room temperature in the presence of a base such as alkaline metal (Na, K) bicarbonate in a solvent system such as THF, DMF, or DMF-H₂O to give the intermediate 11. The ester 11 is converted into the primary amide 12 according to Method A.

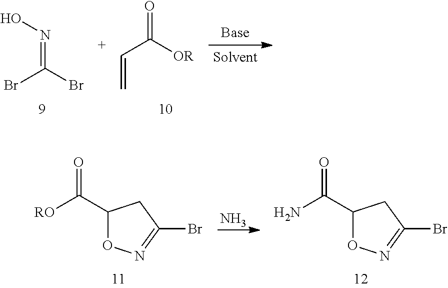

### Method D:

The intermediate 12 is dehydrated with TFAA and tetrazole 13 is obtained following procedures shown in Method A. Alkylation of the tetrazole 13 to give intermediate 14 is also achieved according to Method B.

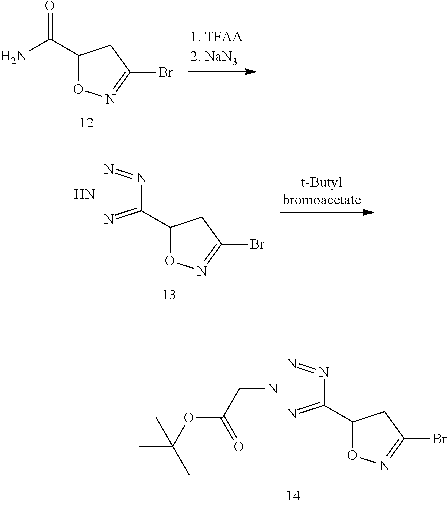

### Method E:

The dihalogenated (X═Cl, Br) pyrimidine 15 is reacted with benzylamine in the presence of a base such as DIPEA in an alcoholic solvent such as 2-propanol. The bromide 16 is reacted with CuCN in the presence of a solvent such as DMF or NMP at a temperature range of about room temperature to about reflux temperature. The intermediate 17 is converted into 18 according to Method A. The benzylamine 18 is cleaved in the presence of an oxidant such as DDQ or CAN and the resulting amine is reacted with SbCl₃ to give the chloride 19.

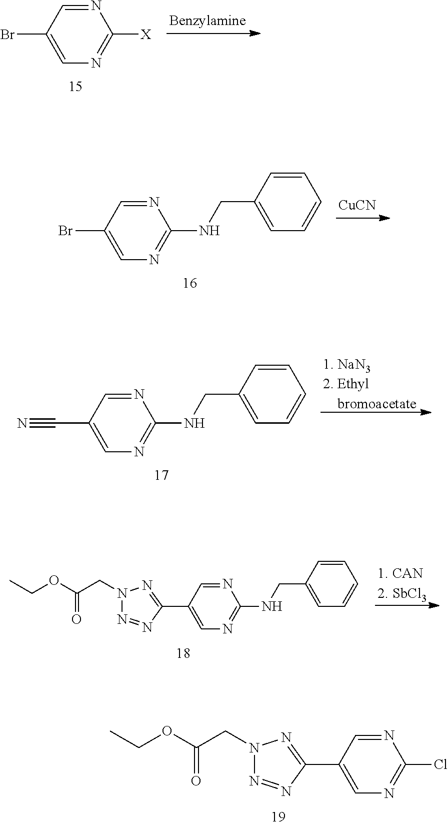

### Method F:

The pyrimidine 20 is reacted with tert-butyl piperazine-1-carboxylate according to the first step of Method E. The 2-alkylated tetrazole 23 is obtained by first forming the nitrile intermediate 22, then the tetrazole, followed by alkylation and separation by chromatography according to Method E. Lastly, the Boc group is cleaved in the presence of a protic acid such as HCl in a solvent such as THF or dioxane.

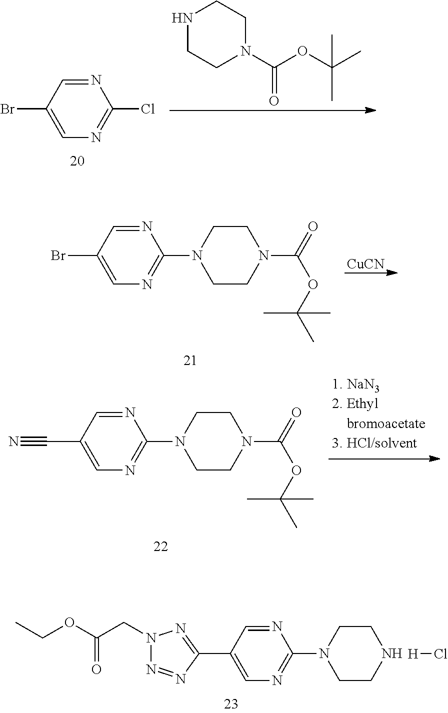

### Method G:

The intermediate 24 is reacted with tert-butyl piperazine-1-carboxylate with a base such as an alkaline metal (Na, K) carbonate in a solvent such as THF or dioxane at a temperature range of room temperature to refluxing temperature to give 25. The ethyl ester is cleaved to the corresponding carboxylic acid with an alkaline metal (Li, Na, K) hydroxide in a solvent system such as MeOH—H₂O or THF—H₂O. The carboxylic acid is then reacted with (COCl₂ or SOCl₂ in a solvent such as toluene or CH₂Cl₂ with a catalytic amount of DMF. The resulting acid chloride is reacted with concentrated ammonium hydroxide in a solvent such as THF or dioxane to give intermediate 26. The intermediate 26 is dehydrated with TFAA to a nitrile, the alkylated tetrazole is elaborated, and the Boc group is cleaved following procedures described in Method F to give intermediate 27.

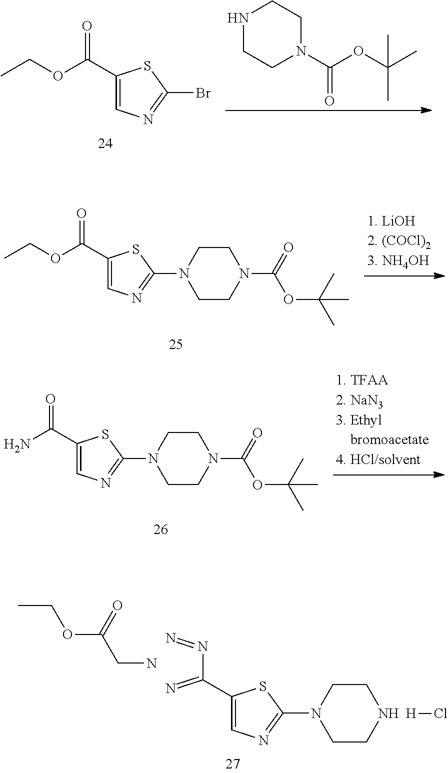

### Method H:

2-Amino-1,3,4-thiadiazole (28) is reacted with bromine in the presence of a base such as sodium acetate in a solvent such as acetic acid to give intermediate 29. The intermediate 30 is obtained following a diazotation with t-butyl nitrite in the presence of CuCN in a solvent such as acetonitrile. The intermediate 30 is reacted with tert-butyl piperazine-1-carboxylate with a base such as DIPEA in a solvent such as THF or dioxane to give nitrile 31. The nitrile is then reacted following procedures shown in Method F to give the 2-alkylated tetrazole hydrochloride salt intermediate 32.

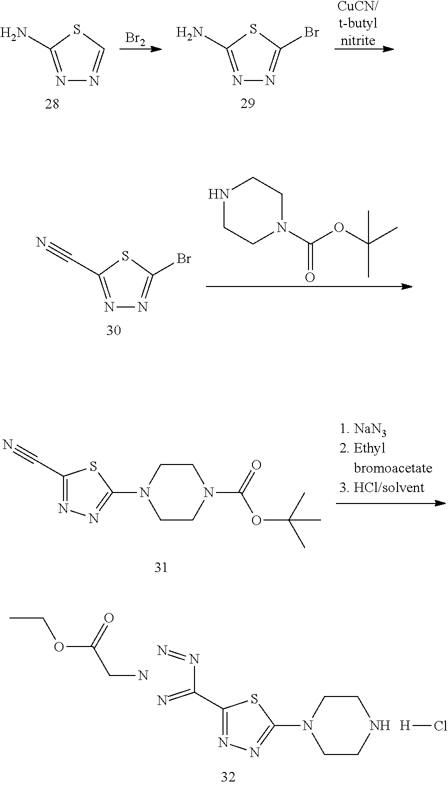

### Method I:

The intermediate 12 is reacted with benzyl piperazine-1-carboxylate in the presence of a base such as DIPEA in an alcoholic solvent such as EtOH or 1-propanol at a temperature range of about room temperature to about reflux temperature to give the intermediate 33. The isoxazole intermediate 34 is obtained by oxidation with iodine in the presence of sodium acetate. Intermediate 34 is further processed following procedures shown in Method G to give intermediate 35. Piperazine 36 is obtained by hydrogenation with Pd/C in an alcoholic solvent such as EtOH.

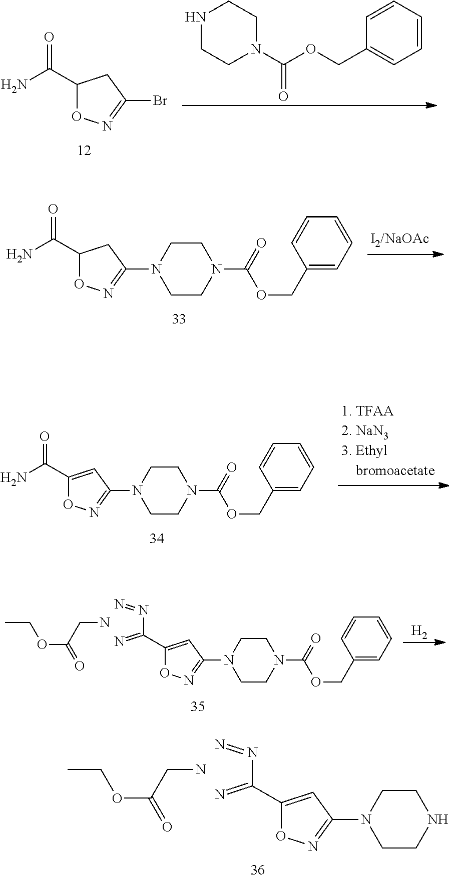

### Method J:

2-Chloropyrazine 37 is reacted with tert-butyl piperazine-1-carboxylate with a base such as an alkaline metal (Na, K, Cs) carbonate and a solvent system such as dioxane, DMF, dioxane-DMF to give the intermediate 38. The intermediate 38 is reacted with NBS in CH₂Cl₂ to give the intermediate 39. The nitrile intermediate 40 is obtained by reacting 39 with CuCN in a solvent such as DMF or NMP at a temperature range of room temperature to reflux temperature. The intermediate 40 is then converted into intermediate 41 following procedures shown in Method H.

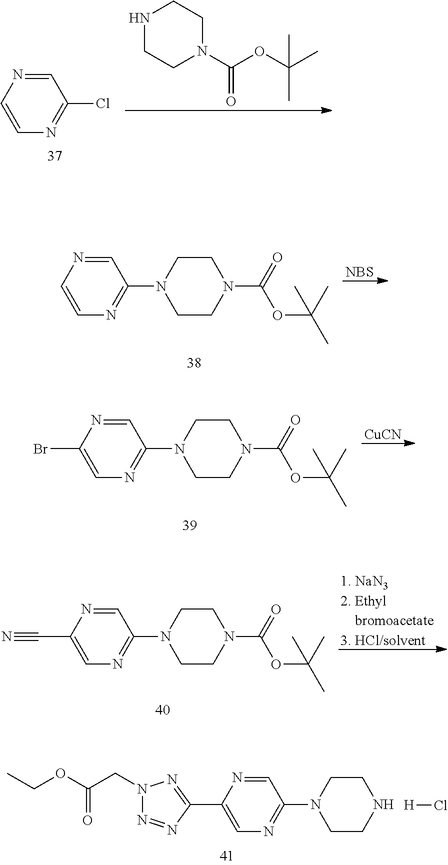

### Method K:

The intermediate 41 is reacted with an appropriately substituted acid chloride in the presence of a base such as Et₃N and a solvent such as CH₂Cl₂ or DMF to give intermediate 42. The carboxylic acid 43 is obtained by reacting the intermediate 42 with an alkali metal (Li, Na, K) hydroxide in a solvent system such as THF—H₂O or MeOH—H₂O.

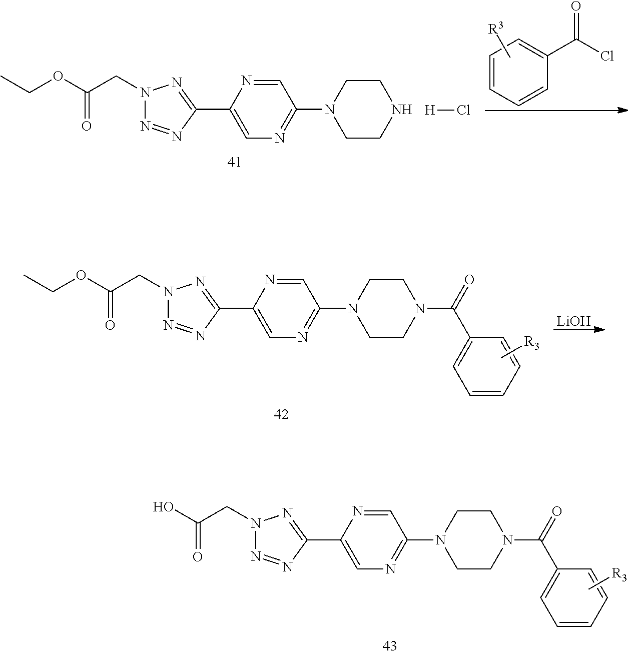

### Method L:

The intermediate 23 is reacted with an appropriately substituted carboxylic acid in the presence of a base such as Et₃N and a coupling agent such as HATU in a solvent such as DMF to give intermediate 44. Hydrolysis of the ester group of the 2-alkylated tetrazole intermediate 44 is carried out according to procedures shown for Method K.

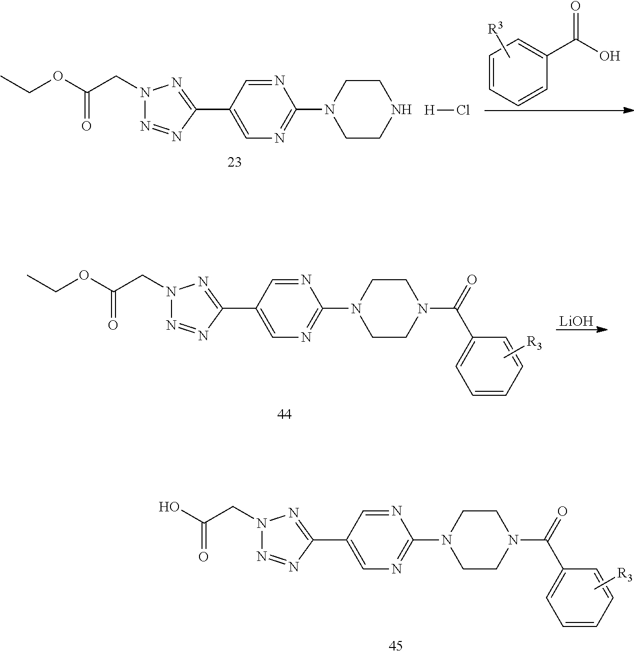

### Method M:

The intermediate 47 is obtained following procedures shown for Method K.

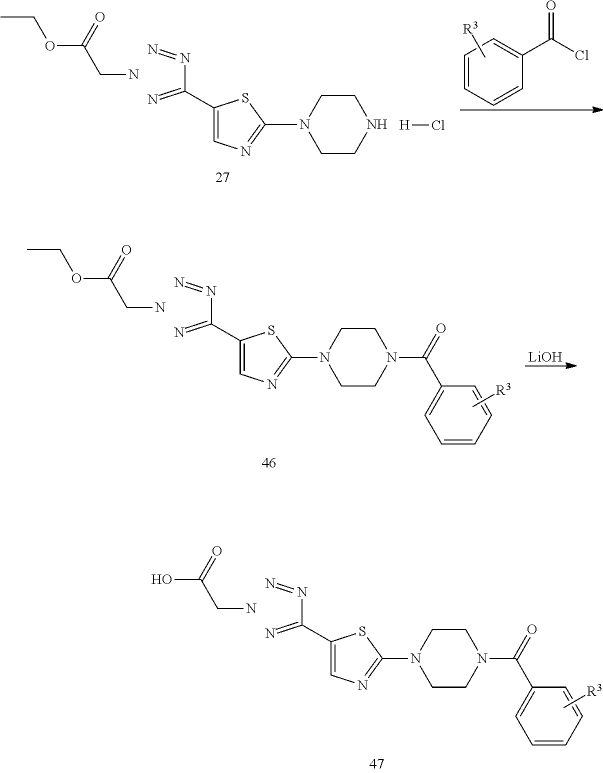

### Method N:

Intermediate 49 is obtained following procedures shown for Method K.

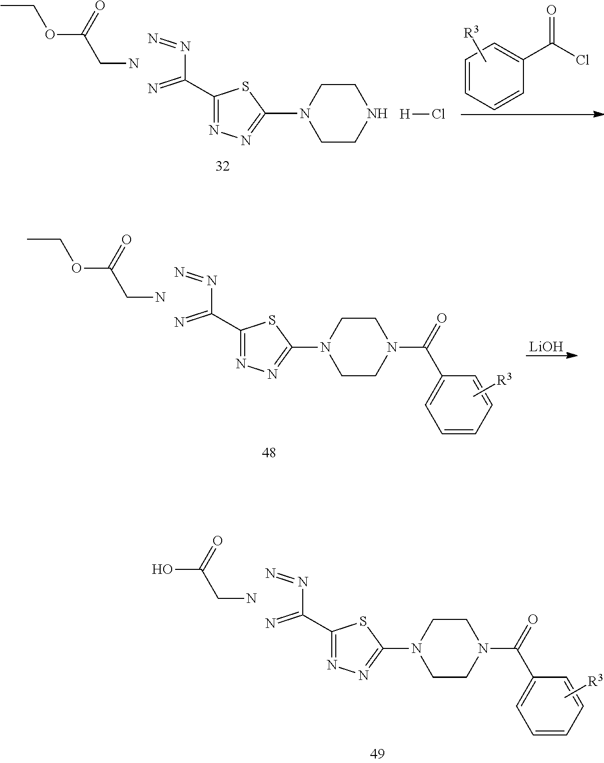

### Method O:

The intermediate 50 is reacted under aryl amination conditions with an appropriately substituted aryl bromide in the presence of a ligand such as BINAP, a catalyst such as palladium(II) acetate and a solvent such as toluene at a temperature range from about room temperature to about reflux temperature to give intermediate 51. The Boc group in 51 is cleaved following procedures for Method H to give intermediate 52.

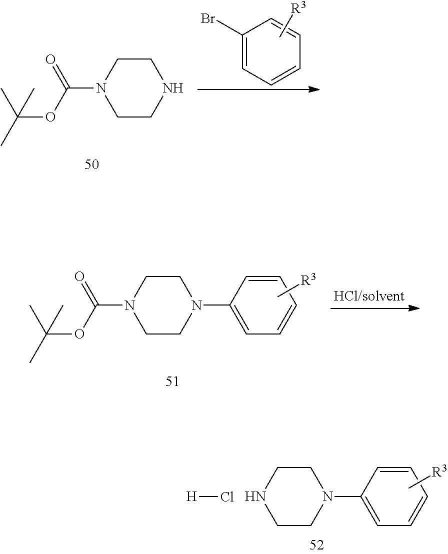

### Method P:

The intermediates 12 and 50 are reacted together in the presence of a base such as an alkali metal (Li, Na, K) carbonate in an alcoholic solvent such as 1-butanol at a temperature range of room temperature to reflux temperature. The isoxazole intermediate 53 is obtained by oxidation with iodine in the presence of a base such as imidazole. The primary amide 53 is reacted following procedures shown for Methods G and D to give intermediate 54. The carboxylic acid 55 is obtained by ester cleavage under acidic conditions such as neat formic acid.

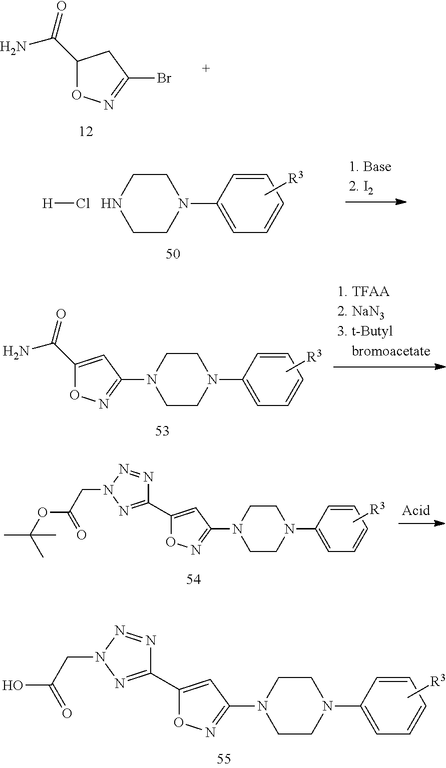

### Method Q:

The Weinreb amide intermediate 56 is reacted with the appropriately substituted aryl bromide in the presence of an alkyllithium such as tert-butyllithium, n-butyllithium or lithium tri-n-butyl magnesate (n-Bu₃MgLi) in a solvent such as THF or Et₂O to give the ketone intermediate 57. The intermediate 57 is reacted following procedures shown for Method H to give intermediate 58.

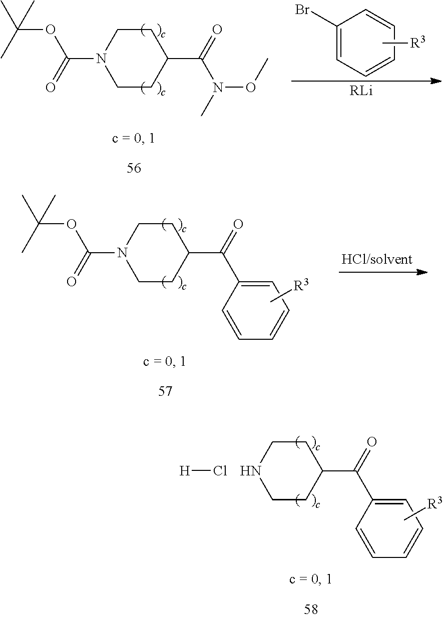

### Method R:

The intermediates 7 and 58 are reacted together in the presence of a base such as DBU in a solvent such as NMP at a temperature range to room temperature to reflux temperature to give intermediate 59. The intermediate 60 is obtained following procedures shown for Method P.

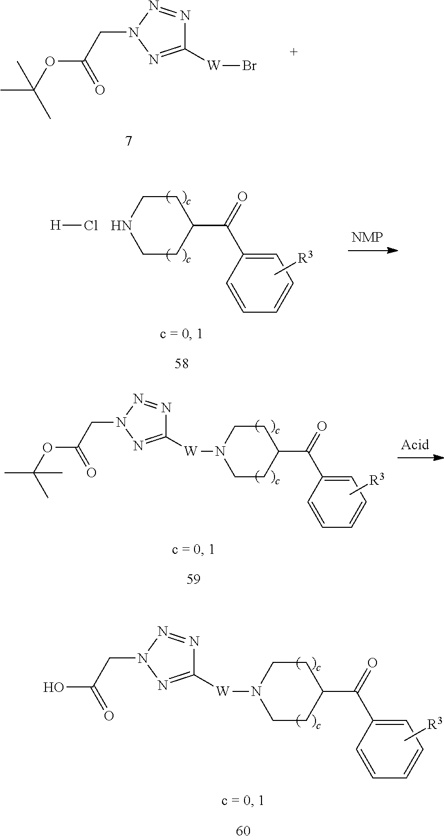

### Method S:

The intermediates 61 and 62 are reacted together in the presence of a catalytic amount of DMAP to give the intermediate 63. The intermediate 63 is reacted with the appropriately substituted boronic acid in the presence of a catalyst such as Pd(OAc)₂ to give the intermediate 57. The intermediate 58 is obtained following procedures shown in Method H. The intermediates 58 and 14 are reacted together in the presence of an alkali metal (Na, K) bicarbonate in a solvent such as t-butanol at a temperature range from about room temperature to about refluxing temperature to give the intermediate M. The isoxazole intermediate 65 is obtained by oxidation of 64 with CAN in a solvent such as THF. The final product 66 is obtained following ester cleavage as shown in Method P.

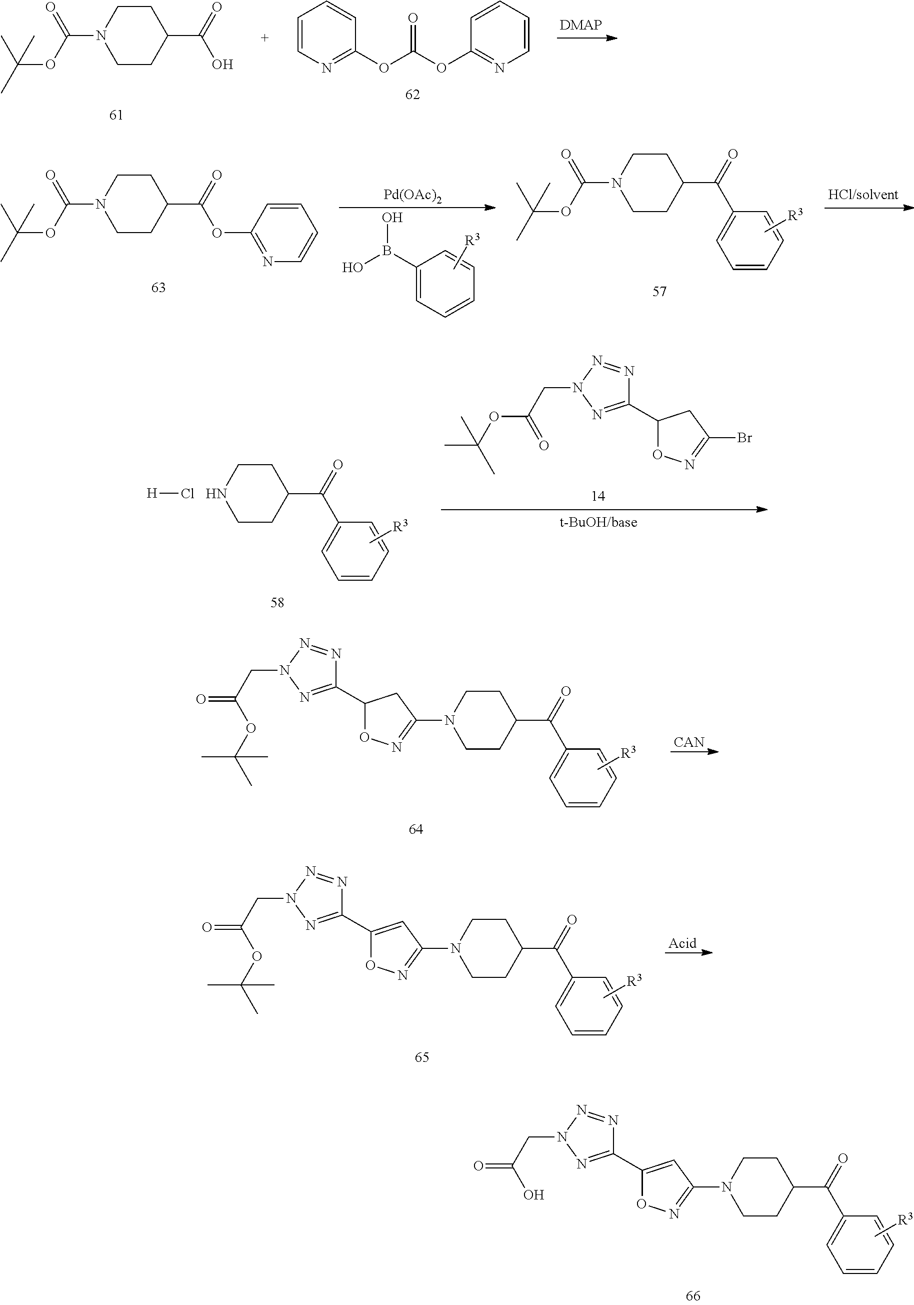

### Method T:

The intermediate 67 is reacted with base such as LDA and N-phenylbis(trifluoromethanesulfonimide) in a solvent such as THF at a temperature range to −78° C. to 0° C. to give the intermediate 68. The intermediate 68 is reacted with an appropriately substituted boronic acid in the presence of a catalyst such as Pd(PPh₃)₄ to give the intermediate 69. The intermediate 69 is converted into intermediate 70 following procedures shown in Method H.

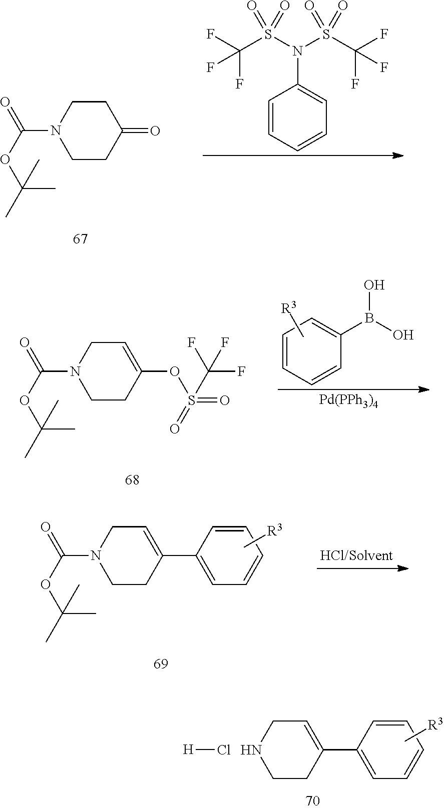

### Method U:

The intermediates 7 and 70 are reacted together following procedures shown in Method R to give the intermediate 71. The intermediate 72 is obtained by ester cleavage as shown in Method P.

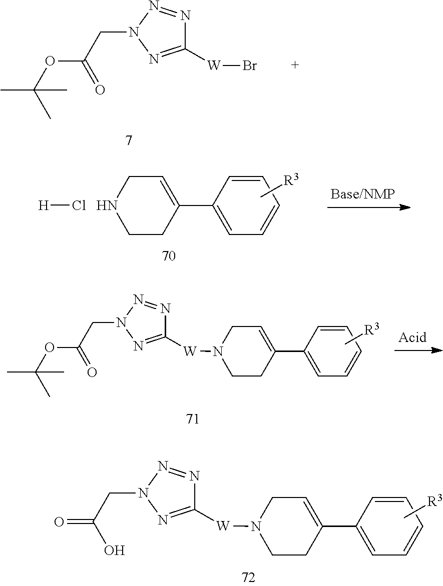

### Method V:

The intermediates 5 and 73 are reacted together following procedures shown in Method R to give the intermediate 74. The carboxylic acid intermediate 75 is obtained by ester cleavage following procedures shown in Method K.

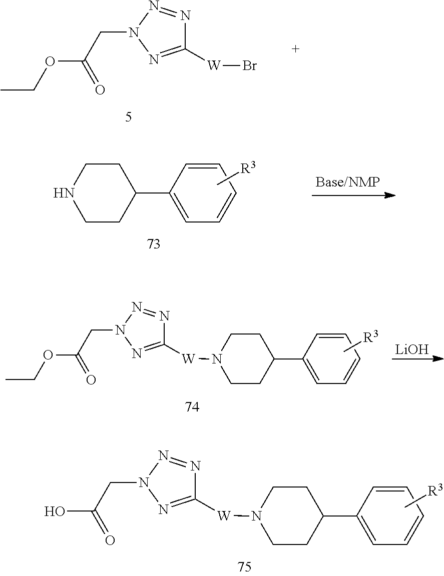

### Method W:

The intermediate 76 is reacted with cyanogen bromide in the presence of a base such as Et₃N in a solvent such as THF at a temperature range of 0° C. to room temperature to give the intermediate 77. The nitrile intermediate 77 is reacted with hydroxylamine in the presence of a base such as Et₃N in an alcoholic solvent such as EtOH to give the intermediate 78. The intermediate 79 is formed by reacting the intermediate 78 with methyl oxalyl chloride followed by reaction with gaseous ammonia. The primary amide is dehydrated according to procedures shown in Method F to give the intermediate 80. The intermediate 81 is obtained following procedures shown in Method G.

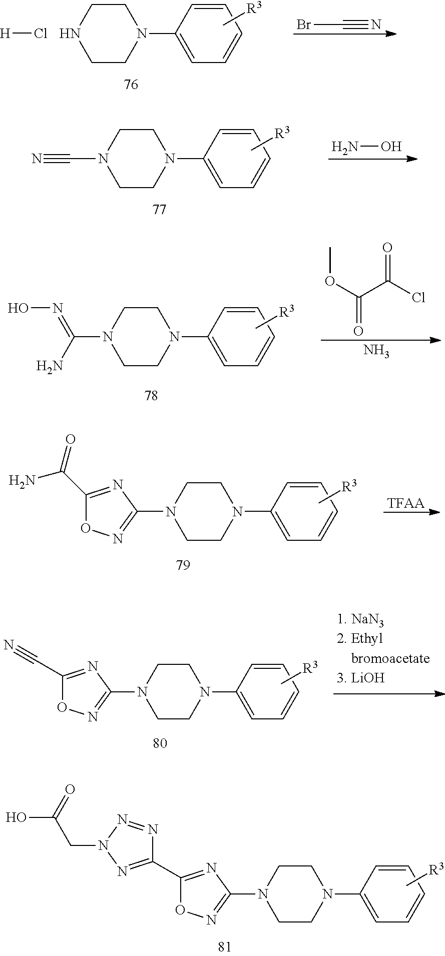

### Method X:

The intermediates 23 and 58 are reacted together in the presence of a base such as an alkali metal (Na, K) carbonate in a solvent such as dioxane at a temperature range to room temperature to refluxing temperature to give the intermediate 82 after cleavage of the ethyl ester following procedures shown in Method K.

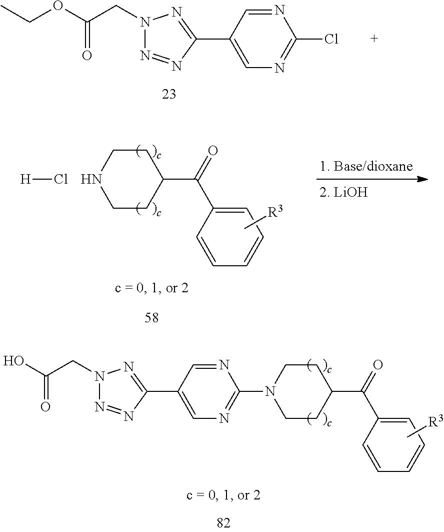

### Method Y:

The intermediate 83 is obtained following procedures shown in Method U.

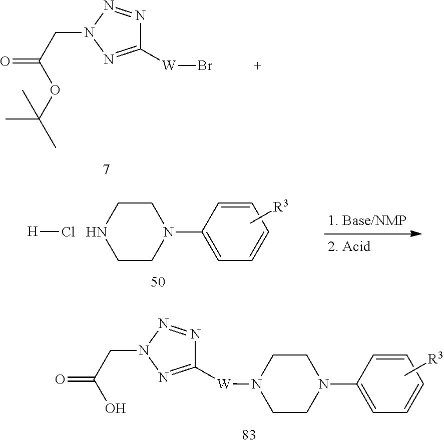

### Method Z:

The Weinreb amide intermediate 56 is reacted with the appropriately substituted aryl bromide in the presence of an alkyllithium such as tert-butyllithium in a solvent such as THF or Et₂O to give the ketone intermediate 57. The intermediate 84 is reacted with bis(pinacolato)diboron in the presence of a Pd catalyst, a phosphine and an inorganic base such as potassium acetate to give intermediate 85. The intermediate 85 is reacted with copper(II) bromide in an alcoholic solvent like methanol and water to provide the aryl bromide 86. The intermediate 86 is reacted following procedures shown for Method H to give intermediate 87.

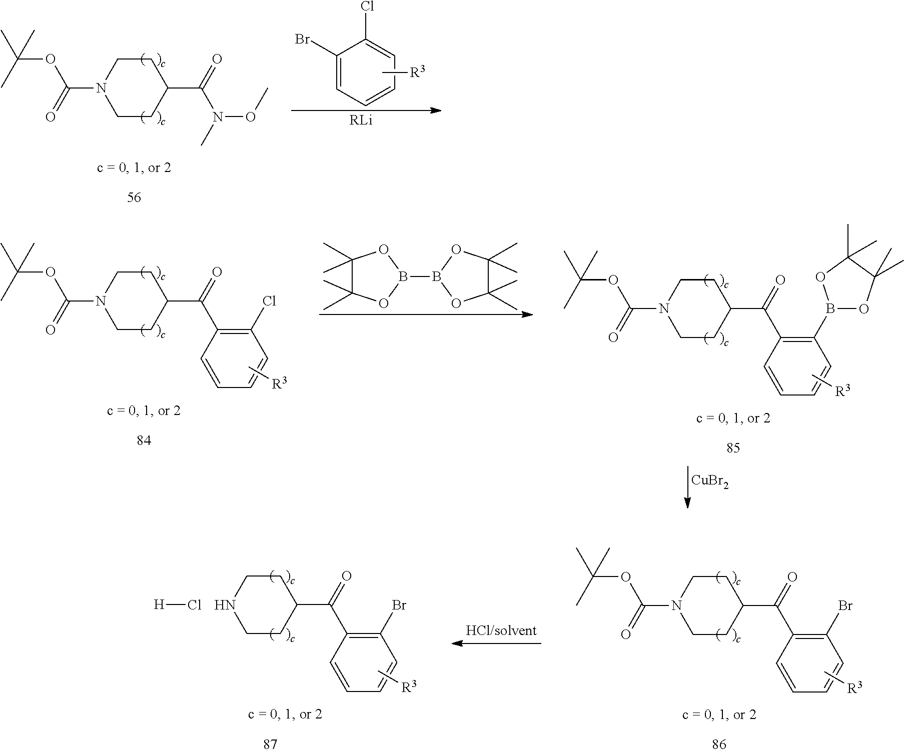

### Method AA:

The aldehyde intermediate 88 is reacted with the appropriately substituted aryl bromide in the presence of an alkyllithium such as tert-butyllithium in a solvent such as THF or Et₂O to give the alcohol intermediate 89. The alcohol intermediate 89 is oxidized to the corresponding ketone with an oxidant such as Dess-Martin periodinane or SO₃. Pyridine to provide the ketone intermediate 57 which is then reacted following procedures shown for Method H to give intermediate 58.

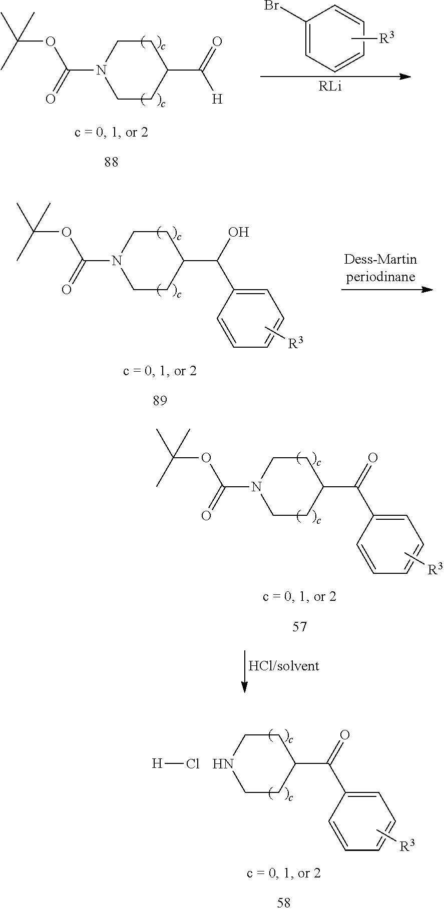

### Method AB:

The chloro intermediate 84 is reacted with a boronic acid or a boroxime in the presence of a palladium catalyst and an inorganic base in a mixture of organic solvents such as toluene or dioxane and water to yield the intermediate 90 which is reacted following procedures shown for Method H to give intermediate 91.

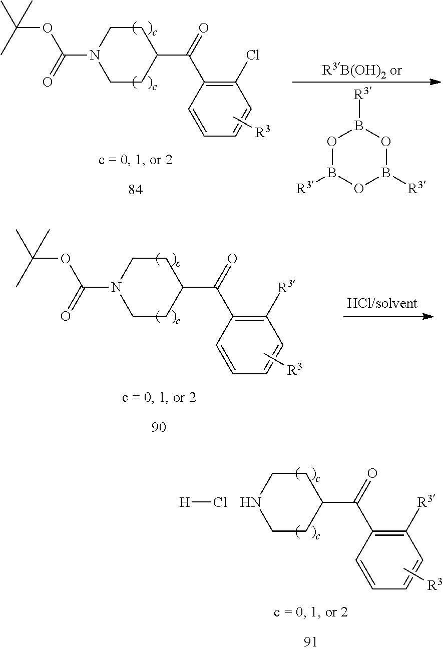

### Method AC:

The appropriately substituted benzoic acid 92 is heated with thionyl chloride or oxalyl chloride to provide the acid chloride intermediate 93. The acid chloride intermediate 93 is reacted with a Grignard reagent in a solvent such as diethyl ether or THF to yield the arylbromide 94. Then, a mixture of dilithium tetrachloromanganate (2-) and a Grignard reagent is reacted with the intermediate 94 to give the appropriately substituted ketone 95. The N-methyl group of 95 is cleaved in an organic solvent such as 1,2-dichloroethane in the presence of a chloroformate like 1-chloroethyl chloroformate. The hydrochloride salt 91 is obtained after addition of an alcoholic solvent like methanol.

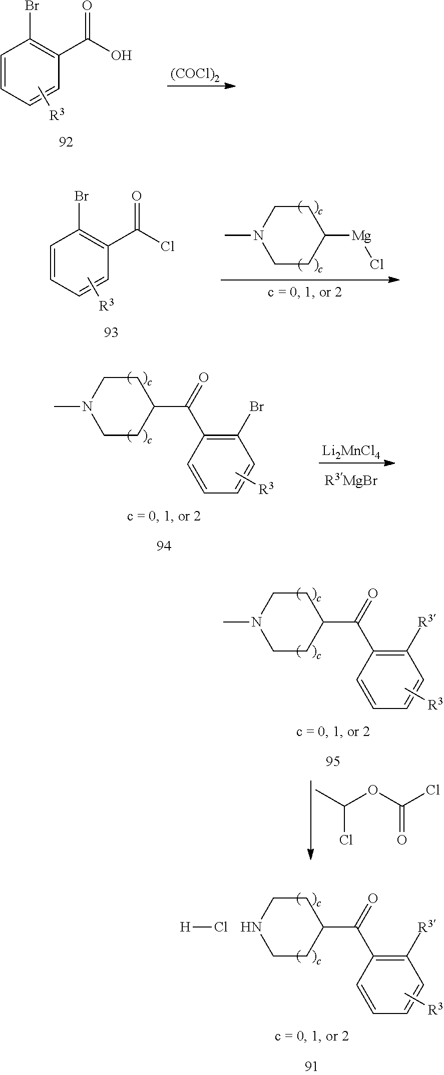

### Method AD:

Alternatively, intermediate 94 is reacted with a mixture formed by zinc chloride and the appropriately substituted Grignard reagent in the presence of a palladium and copper catalyst to give the aryl ketone 95. The N-methyl group of 95 is cleaved as shown for Method AC to give the hydrochloride salt 96.

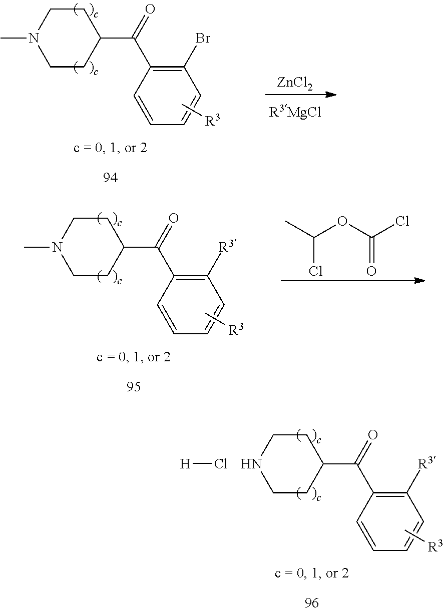

### Preparation of Key Intermediates

### Intermediate 1

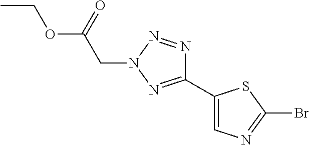

### Ethyl [5-(2-bromo-1,3-thiazol-5-yl)-2H-tetrazol-2-yl]acetate

### Step 1: 2-Bromo-1,3-thiazole-5-carboxamide

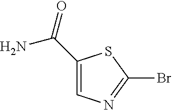

Into a 2 L round-bottom flask was added ethyl 2-bromothiazole-5-carboxylate (50.0 g, 212 mmol), THF (500 mL) and MeOH (250 mL). To this was added concentrated ammonium hydroxide in water (590 mL) and the reaction mixture was stirred at room temperature for 4 h. The solvents were removed under reduced pressure and the crude mixture poured into a separatory funnel containing brine (1 L). The aqueous layer was extracted with EtOAc (4×500 mL) and the combined organic layers were washed with brine, dried over Na₂SO₄, filtered and concentrated under reduced pressure.

### Step 2: 2-Bromo-1,3-thiazole-5-carbonitrile

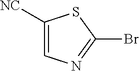

Into a 2 L round-bottom flask containing 2-bromo-1,3-thiazole-5-carboxamide (41.5 g, 201 mmol) in CH₂Cl₂ (1.3 L) was added triethylamine (70 mL, 502 mmol). The resulting solution was cooled to 0° C. and TFAA (34 mL, 241 mmol) was added slowly over 15 min. The reaction mixture was allowed to warm to room temperature and stirred for 2 h. The reaction mixture was poured into a 3 L separatory funnel containing saturated aqueous NaHCO₃ solution (500 mL). The aqueous layer was extracted with CH₂Cl₂ (2×1.2 L) and the combined organic layers were washed with brine, dried over Na₂SO₄, filtered and concentrated under reduced pressure. The crude reaction mixture was filtered through a short plug of silica gel on a sintered glass funnel, washing with copious quantities of EtOAc. The filtrate was concentrated under reduced pressure to provide the title compound.

### Step 3: 5-(2-Bromo-1,3-thiazol-5-yl)-2H-tetrazole

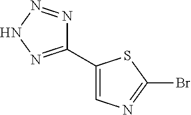

A solution of 2-bromo-1,3-thiazole-5-carbonitrile (5.00 g, 26.5 mmol) in 2-propanol (75 mL) and water (38 mL) was treated with ZnBr₂ (5.96 g, 26.5 mmol) and sodium azide (2.58 g, 39.7 mmol). The reaction mixture was heated at 120° C. for 5 h. The cooled reaction mixture was diluted with water (50 mL) and acidified to pH 3 using aqueous 1 NHCl solution (about 20 mL). The mixture was poured into a 500 mL separatory funnel and the aqueous layer was extracted with EtOAc (4×100 mL). The combined organic layers were washed with brine, dried over Na₂SO₄, filtered and concentrated under reduced pressure to provide the tetrazole compound.

### Step 4: Ethyl [5-(2-bromo-1,3-thiazol-5-yl)-2H-tetrazol-2-yl]acetate

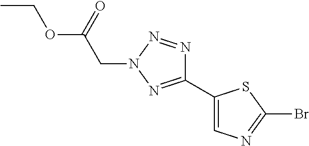

Into a 250 mL round-bottom flask containing 5-(2-bromo-1,3-thiazol-5-yl)-2H-tetrazole (5.43 g, 22.5 mmol) in THF (81 mL) was added triethylamine (7.2 mL, 52 mmol) and ethyl bromoacetate (3.8 mL, 34 mmol). The resulting mixture was heated at 80° C. for 1 h, and then cooled to room temperature. The reaction mixture was poured into a separatory funnel containing water (80 mL) and the aqueous layer was extracted with EtOAc (2×160 mL). The combined organic layers were washed with brine, dried over Na₂SO₄, filtered and concentrated under reduced pressure. Purification by column chromatography through silica gel, eluting with 100% hexanes to 50:50 hexanes:EtOAc as a gradient provided the desired alkylated tetrazole as a single regioisomer.

¹H NMR (d₆-DMSO, 400 MHz): δ 8.39 (1H, s), 5.93 (2H, s), 4.21 (2H, q, J=7.0 Hz), 1.22 (3H, t, J=7.0 Hz).

### Intermediate 2

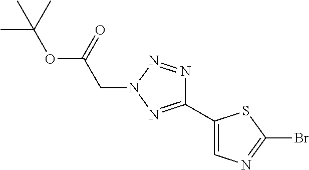

### tert-Butyl [5-(2-bromo-1,3-thiazol-5-yl)-2H-tetrazol-2-yl]acetate

This compound was synthesized in a similar manner as ethyl [5-(2-bromo-1,3-thiazol-5-yl)-2H-tetrazol-2-yl]acetate (Intermediate 1) using tert-butyl bromoacetate in place of ethyl bromoacetate.

¹H NMR (CDCl₃, 400 MHz): δ 8.22 (1H, s), 5.32 (2H, s), 1.47 (9H, s).

MS (ESI, Q⁺) m/z 346, 348 (M+1, ⁷⁹Br, ⁸¹Br).

### Intermediate 3

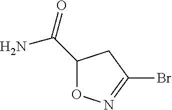

### 3-Bromo-4,5-dihydroisoxazole-5-carboxamide

### Step 1: Ethyl 3-bromo-4,5-dihydroisoxazole-5-carboxylate

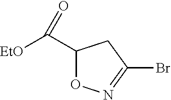

To a round-bottom flask containing hydroxycarbonimidic dibromide (100 g, 490 mmol) was slowly added DMF (300 mL) followed by ethyl acrylate (59 g, 590 mmol). The mixture was cooled to −10° C. and then a solution of KHCO₃ (99 g, 990 mmol) in water (400 mL) was added dropwise over 90 min, at a rate which maintained the internal temperature below 0° C. Stirring was continued at 0° C. for 1.5 h. The reaction mixture was poured into a 4 L separatory funnel containing water (500 mL) and the aqueous layer was extracted with MTBE (3×500 mL). The combined organic layers were washed with brine, dried over MgSO₄, filtered and concentrated under reduced pressure to give a yellow oil which was used directly in Step 2.

### Step 2: 3-Bromo-4,5-dihydroisoxazole-5-carboxamide

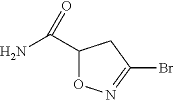

Ethyl 3-bromo-4,5-dihydroisoxazole-5-carboxylate (109 g, 490 mmol) was added to a 1 L round-bottom flask containing 2.0 M NH₃ in MeOH (295 mL). The reaction mixture was heated at 50° C. for 2.5 h and then cooled to room temperature and stirred overnight for 16 h. The resulting slurry was diluted with 500 mL of diethyl ether and stirred in an ice-bath for 1 h. The product was isolated by filtration under vacuum, affording the title compound as a tan solid. ¹H NMR (CDCl₃, 400 MHz): δ 6.70 (1H, bs), 5.92 (1H, bs), 5.06 (1H, dd, J=11.0, 6.5 Hz), 3.64-3.51 (2H, m). MS (ESI, Q⁺) m/z 193, 195 (M+1, ⁷⁹Br, ⁸¹Br).

### Intermediate 4

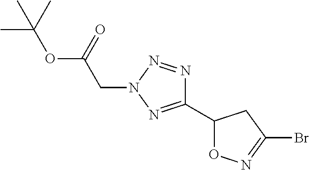

### tert-Butyl [5-(3-bromo-4,5-dihydroisoxazol-5-yl)-2H-tetrazol-2-yl]acetate

### Step 1: 3-Bromo-4,5-dihydroisoxazole-5-carbonitrile

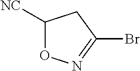

To a solution of 3-bromo-4,5-dihydroisoxazole-5-carboxamide (Intermediate 3, 30.0 g, 155 mmol) in THF (360 mL) was added triethylamine (43.0 mL, 311 mmol). The solution was cooled to 0° C. and TFAA (33.0 mL, 233 mmol) was added slowly over 20 min, at a rate which maintained the internal temperature below 15° C. The reaction mixture was stirred at 0° C. for 1 h. The reaction mixture was poured into a 2 L separatory funnel containing water (500 mL) and the aqueous layer was extracted with MTBE (3×500 mL). The combined organic layers were washed with a saturated aqueous NaHCO₃ solution (2×250 mL), brine, dried over MgSO₄, filtered and concentrated under reduced pressure to afford the title compound.

### Step 2: 5-(3-Bromo-4,5-dihydroisoxazol-5-yl)-2H-tetrazole

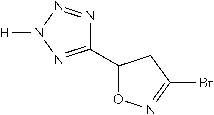

Into a 2 L round-bottom flask equipped with a reflux condenser, heating mantle and under N₂ was added 3-bromo-4,5-dihydroisoxazole-5-carbonitrile (39.4 g, 225 mmol), zinc oxide (1.8 g, 23 mmol), THF (40 mL) and water (200 mL). To this solution was added in slowly a solution of sodium azide (16 g, 250 mmol) in water (10 mL) over 5 min and the mixture was warmed to 75° C. for 16 h. Heating was applied at a rate in where the internal temperature of the reaction mixture did not exceed 80° C. The reaction mixture was cooled to 0° C. and acidified to pH 3-4 with slow addition of 2 N aqueous HCl solution. During the acidification, the internal temperature was maintained below 5° C. The reaction mixture was poured into a 2 L separatory funnel and the aqueous layer was extracted with EtOAc (3×500 mL). The combined organic layers were washed with brine, dried over MgSO₄, filtered and concentrated under reduced pressure to afford the title compound.

### Step 3: tert-Butyl [5-(3-bromo-4,5-dihydroisoxazol-5-yl)-2H-tetrazol-2-yl]acetate

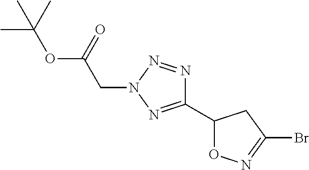

Into a 2 L round-bottom flask equipped with a reflux condenser, heating mantle and under N₂ was added 5-(3-bromo-4,5-dihydroisoxazol-5-yl)-2H-tetrazole (49 g, 225 mmol) and THF (500 mL). Triethylamine (53 mL, 383 mmol) was added to the mixture and the solution heated to 55° C. while tert-butyl bromoacetate (66 g, 338 mmol) was added. The mixture was heated at 55° C. for 1 h and then cooled to room temperature. The reaction mixture was poured into a 2 L separatory funnel containing 1 N aqueous HCl solution (500 mL) and the aqueous layer was extracted with EtOAc (3×500 mL). The combined organic layers were washed with brine, dried over MgSO₄, filtered and concentrated under reduced pressure. Purification by column chromatography through iatrobead silica gel, eluting with 75:15:5 hexanes:EtOAc:CH₂Cl₂, afforded the title product in a greater than 10:1 regioisomeric purity. ¹H NMR (CDCl₃, 400 MHz): δ 5.98 (1H, dd, J=11.0, 7.5 Hz), 5.35 (2H, s), 3.87 (1H, dd, J=17.5, 7.5 Hz), 3.70 (1H, dd, J=17.5, 11.0 Hz), 1.50 (9H, s).

MS (ESI, Q⁺) m/z 332, 334 (M+1, ⁷⁹Br, ⁸¹Br).

### Intermediate 5

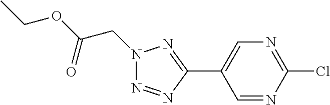

### Ethyl [5-(2-chloropyrimidin-5-yl)-2H-tetrazol-2-yl]acetate

### Step 1: N-Benzyl-5-bromopyrimidin-2-amine

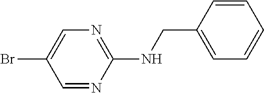

Into a 2 L round-bottom flask equipped with a heating mantle, reflux condenser and under N₂ was added 2-chloro-5-bromopyrimidine (125 g, 646 mmol), DIPEA (251 mL, 1435 mmol) and benzylamine (95 mL, 872 mmol) in 2-propanol (250 mL). The reaction mixture was heated to 100° C. for 1 h and then cooled to room temperature and stirred for 16 h. The crude reaction mixture was filtered under vacuum on a sintered glass funnel, and the filter cake was rinsed with ethanol (2×50 mL) and hexanes (200 mL). The filter cake was further dried under vacuum to provide the title compound as a white crystalline solid.

### Step 2: 2-(Benzylamino)pyrimidine-5-carbonitrile

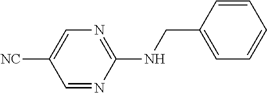

Into a 5 L round-bottom flask equipped with a reflux condenser and heating mantle and under N₂ was added N-benzyl-5-bromopyrimidin-2-amine (150 g, 568 mmol), copper(I) cyanide (64 g, 710 mmol) and DMF (1.5 L). The reaction mixture was heated to 150° C. for 16 h. The reaction mixture was cooled to room temperature and poured into a 3 L separatory funnel containing 750 mL of a 1:1:2 aqueous solution of saturated NH₄Cl:concentrated NH₄OH:water. The aqueous layer was extracted with MeTHF (3×500 mL) and the combined organic layers were washed with brine, dried over Na₂SO₄, filtered and concentrated under reduced pressure. The obtained product was utilized in the subsequent step without further purification.

### Step 3: N-Benzyl-5-(2H-tetrazol-5-yl)pyrimidin-2-amine

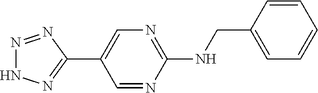

A suspension of 2-(benzylamino)pyrimidine-5-carbonitrile (34 g, 162 mmol), sodium azide (13 g, 202 mmol) and ammonium chloride (35 g, 647 mmol) in DMF (340 mL) was heated at 100° C. A steady flow of N₂ (170 mL/min) was placed above the reaction mixture and the reaction flask was kept open and well-vented. At t=1.5 h, t=3 h and t=4 h, an additional 1 equiv of sodium azide (10.5 g, 162 mmol) was added to the mixture. After 5 h total reaction time, the mixture was allowed to cool to room temperature. The reaction was poured into a 2 L separatory funnel containing aqueous 1 N NaOH solution (750 mL) and the aqueous layer was extracted with MTBE (2×200 mL). The aqueous layer was cooled to 0° C. in an ice bath and acidified to pH 1-2 with aqueous 2 M HCl solution. During the acidification, the internal temperature was maintained below 15° C. The aqueous mixture was poured into a separatory funnel and extracted with EtOAc (3×300 mL). The combined organic layers were washed with brine, dried over Na₂SO₄, filtered and concentrated under reduced pressure to afford to the title compound as a beige solid.

### Step 4: Ethyl {5-[2-(benzylamino)pyrimidin-5-yl]-2H-tetrazol-2-yl}acetate

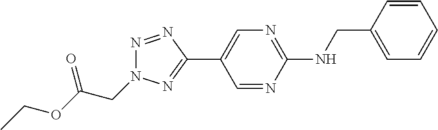

To a 2 L round-bottom flask equipped with a heating mantle and reflux condenser was added N-benzyl-5-(2H-tetrazol-5-yl)pyrimidin-2-amine (31.9 g, 126 mmol), ethyl bromoacetate (21 mL, 188 mmol), triethylamine (35 mL, 251 mmol) and THF (390 mL). The reaction mixture was heated to 65° C. for 1 h and then cooled to room temperature. Water (1 L) was added and the mixture was stirred at room temperature for 1 h, then filtered under vacuum on a sintered glass funnel. The filter cake was further washed with water:THF (2.5:1, 300 mL) and then with water (500 mL). The resulting cake was re-suspended in THF (320 mL) and then water (640 mL) was added gradually over 0.5 h. The suspension was stirred an additional 0.5 h at room temperature and then filtered under vacuum on a sintered glass funnel. The filter cake was washed with 2:1 water:THF (2×200 mL) and dried under vacuum for several hours, affording the title compound as white powder.

### Step 5: Ethyl[5-(2-aminopyrimidin-5-yl)-2H-tetrazol-2-yl]acetate

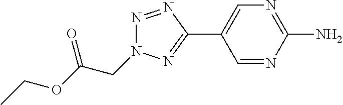

Into a 1 L round-bottom flask was dissolved ethyl {5-[2-(benzylamino)pyrimidin-5-yl]-2H-tetrazol-2-yl}acetate (30.7 g, 90 mmol) in MeCN (300 mL) and water (60 mL). To this solution was added cerium ammonium nitrate (114 g, 208 mmol) portion wise over 15 min. The mixture was stirred at room temperature for 1 h and was poured into a separatory funnel containing water (500 mL). The aqueous layer was extracted with EtOAc (3×250 mL). The combined organic layers were washed with aqueous 0.1 N HCl solution/brine (1:1; 250 mL), brine, dried over Na₂SO₄, filtered and concentrated under reduced pressure to afford the title compound.

### Step 6: Ethyl [5-(2-chloropyrimidin-5-yl)-2H-tetrazol-2-yl]acetate

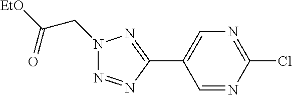

A solution of ethyl [5-(2-aminopyrimidin-5-yl)-2H-tetrazol-2-yl]acetate (16.6 g, 66 mmol) in 1,2-dichloroethane (330 mL) was treated with antimony(III) chloride (19.3 mL, 266 mmol). The mixture was cooled to 0° C. in an ice bath and tert-butyl nitrite (44 mL, 332 mmol) was added dropwise to the reaction mixture over 15 min. After 3 h, the mixture was diluted with saturated aqueous NaHCO₃ solution (200 mL) and CH₂Cl₂ (200 mL) and the resulting suspension was filtered through a pad of celite on a sintered glass funnel under vacuum. The filtrate was poured into a 2 L separatory funnel containing saturated aqueous NaHCO₃ solution (250 mL) and the aqueous layer was extracted with CH₂Cl₂ (3×200 mL). The combined organic layers were washed with brine, dried over Na₂SO₄, filtered and concentrated under reduced pressure. Purification by column chromatography through silica gel, eluting with 85:15 hexanes:EtOAc to 50:50 hexanes:EtOAc as a gradient afforded the title compound as an off-white solid.

¹H NMR (d₆-DMSO, 400 MHz): δ 9.40 (2H, s), 6.01 (2H, s), 4.24 (2H, q, J=7.0 Hz), 1.25 (3H, t, J=7.0 Hz). MS (ESI, Q⁺) m/z 269, 271 (M+1, ³⁵C1, ³⁷C1).

### Intermediate 6

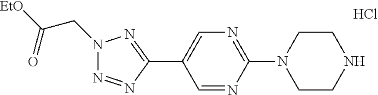

### Ethyl {5-[2-(piperazin-1-yl)pyrimidin-5-yl]-2H-tetrazol-2-yl}acetate Hydrochloride

### Step 1: tent-Butyl 4-(5-bromopyrimidin-2-yl)piperazine-1-carboxylate

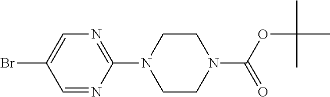

Into a 200 mL pressure flask equipped with a magnetic stir bar was added tert-butyl piperazine-1-carboxylate (4.8 g, 25.8 mmol), 5-bromo-2-chloropyrimidine (5.0 g, 25.8 mmol) and 2-propanol (50 mL). DIPEA (5.0 mL, 28.4 mmol) was added, the vial was sealed and the reaction mixture heated to 120° C. for 1 h. The cooled reaction mixture was poured into a 250 mL separatory funnel containing water (125 mL) and extracted with EtOAc (3×75 mL). The combined organic layers were washed with brine, dried over MgSO₄, filtered and concentrated. Recrystallization from EtOAc (about 40 mL) and hexanes (about 150 mL) at −78° C. afforded crystals which were collected by filtration through filter paper on a Hirsch funnel under vacuum.

### Step 2: tert-Butyl 4-(5-cyanopyrimidin-2-yl)piperazine-1-carboxylate

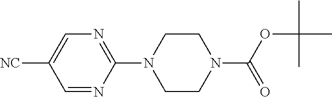

Into a 200 mL pressure flask equipped with a magnetic stir bar was added tert-butyl 4-(5-bromopyrimidin-2-yl)piperazine-1-carboxylate (5.0 g, 14.6 mmol) and DMF (73 mL). Copper(I) cyanide (2.6 g, 29.0 mmol) was added and the flask was sealed and heated to 140° C. for 19 h. The reaction mixture was diluted with water (100 mL) and EtOAc (75 mL) and filtered through a short plug of celite on a sintered glass funnel under vacuum. The filtrate was poured into a 250 mL separatory funnel containing water (50 mL) and the aqueous layer was extracted with EtOAc (3×75 mL). The combined organic layers were washed with brine, dried over MgSO₄, filtered and concentrated under reduced pressure. Purification by column chromatography through silica gel, eluting with 0% EtOAc in hexanes to 40% EtOAc in hexanes as a gradient provided the desired compound. MS (ESI, Q⁺) m/z 312 (M+Na).

### Step 3: tert-Butyl 4-[5-(2H-tetrazol-5-yl)pyrimidin-2-yl]piperazine-1-carboxylate

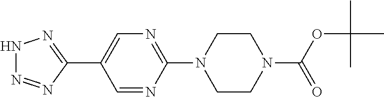

Into a 100 mL pressure flask equipped with a magnetic stir bar was added tert-butyl 4-(5-cyanopyrimidin-2-yl)piperazine-1-carboxylate (1.43 g, 4.93 mmol), sodium azide (640 mg, 9.86 mmol) and ammonium chloride (790 mg, 14.8 mmol) in DMF (40 mL). The vial was sealed and the reaction mixture was heated to 130° C. for 19 h. The cooled reaction mixture was poured into a 250 mL separatory funnel containing 1 N aqueous NaOH solution (100 mL) and washed with diethyl ether (2×50 mL). The aqueous layer was acidified to pH 1 with concentrated HCl and extracted with EtOAc (3×50 mL). The combined organic layers were washed with brine, dried over MgSO₄, filtered and concentrated to provide the desired compound as a solid.

### Step 4: tert-Butyl 4-{5-[2-(2-ethoxy-2-oxoethyl)-2H-tetrazol-5-yl]pyrimidin-2-yl}piperazine-1-carboxylate

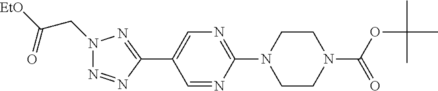

Into a 50 mL pressure tube equipped with a magnetic stir bar was added tert-butyl 4-[5-(2H-tetrazol-5-yl)pyrimidin-2-yl]piperazine-1-carboxylate (1.55 g, 4.66 mmol), ethyl bromoacetate (0.78 mL, 7.00 mmol) and triethylamine (0.95 mL, 9.33 mmol) and THF (24 mL). The reaction mixture was heated to 80° C. for 1 h, cooled to room temperature and the solvent removed under vacuum. The reaction mixture was dissolved in a minimal amount of CH₂Cl₂ with gentle heating and then MeOH was added until precipitation occurred. The suspension was cooled to −78° C. for 15 min and then filtered through filter paper. The resulting white solid was washed with MeOH (2 mL), to afford the title compound.

### Step 5: Ethyl {5-[2-(piperazin-1-yl)pyrimidin-5-yl]-2H-tetrazol-2-yl}acetate Hydrochloride

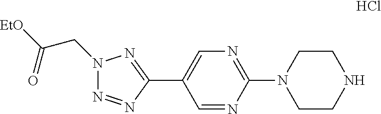

Into a 100 mL round-bottom flask equipped with a magnetic stir bar was added tert-butyl 4-{5-[2-(2-ethoxy-2-oxoethyl)-2H-tetrazol-5-yl]pyrimidin-2-yl}piperazine-1-carboxylate (800 mg, 1.91 mmol) and 4.0 M hydrochloric acid in dioxane (4.8 mL, 19.1 mmol).

The resulting solution was stirred at room temperature for 16 h, becoming a white suspension. The suspension was filtered through filter paper on a Hirsch funnel, washing with diethyl ether (5 mL) to afford the desired product as a white solid.

¹H NMR (CD₃OD, 400 MHz): δ 7.55 (2H, s), 4.17 (2H, s), 2.76 (2H, q, J=7.0 Hz), 2.69-2.67 (4H, m), 1.80-1.78 (4H, m), −0.21 (3H, t, J=7.0 Hz). MS (ESI, Q) m/z 319 (M+1).

### Intermediate 7

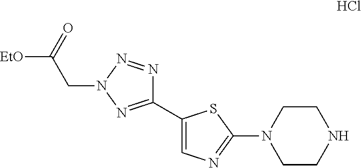

### Ethyl {5-[2-(piperazin-1-yl)-1,3-thiazol-5-yl]-2H-tetrazol-2-yl}acetate hydrochloride

### Step 1: tert-Butyl 4-[5-(ethoxycarbonyl)-1,3-thiazol-2-yl]piperazine-1-carboxylate

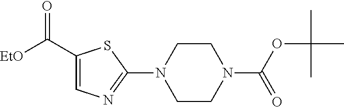

Into a 100 mL pressure flask equipped with a magnetic stir bar was added ethyl 5-carboxylate 2-bromothiazole (6.00 g, 25.4 mmol), tert-butyl piperazine-1-carboxylate (4.75 g, 25.4 mmol) and potassium carbonate (5.27 g, 38.1 mmol). The solids were suspended in dioxane (20 mL) and the vial was sealed and heated to 90° C. for 16 h. The resulting suspension was cooled to room temperature and diluted with water (75 mL). The mixture was stirred at room temperature for 15 min and filtered through filter paper on a Hirsch funnel, washing with water (5 mL). The title compound was obtained as a light yellow solid.

### Step 2: tert-Butyl 4-(5-carbamoyl-1,3-thiazol-2-yl)piperazine-1-carboxylate

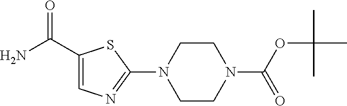

Into a 250 mL round-bottom flask equipped with a magnetic stir bar was added tert-butyl 4-[5-(ethoxycarbonyl)-1,3-thiazol-2-yl]piperazine-1-carboxylate (3.00 g, 8.79 mmol) and THF (75 mL). The solution was treated with 1 N aqueous LiOH solution (17.5 mL, 17.5 mmol) and stirred at room temperature for 6 h until complete conversion of starting material was observed. The reaction mixture was concentrated under reduced pressure to remove the THF and then acidified to pH 4 with 1 N aqueous HCl. The resulting suspension was poured into a 250 mL separatory funnel and extracted with EtOAc (3×50 mL). The combined organic layers were washed with brine, dried over MgSO₄, filtered and the solvent was evaporated under reduced pressure to give an off-white solid. The crude carboxylic acid was placed into a 250 mL round-bottom flask equipped with a magnetic stir bar and containing DMF (0.14 mL, 1.76 mmol) and CH₂Cl₂ (75 mL). The suspension was treated with dropwise addition of oxalyl chloride (0.85 mL, 9.7 mmol) and stirred at room temperature for 30 min. The reaction mixture was concentrated under reduced pressure to remove excess oxalyl chloride and dichloromethane and the residue was dissolved in THF (75 mL). The suspension was treated with concentrated NH₄OH (1.7 mL, 44 mmol) and stirred at room temperature for 16 h, becoming a white suspension. The reaction mixture was poured into a 500 mL separatory funnel containing water (75 mL) and the mixture was extracted with EtOAc (3×125 mL). The combined organic layers were washed with brine, dried over MgSO₄, filtered and the solvent was evaporated under reduced pressure. The desired product was obtained as an off-white solid.

MS (ESI, Q⁺) m/z 313 (M+1).

### Step 3: tert-Butyl 4-(5-cyano-1,3-thiazol-2-yl)piperazine-1-carboxylate

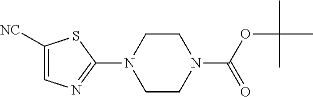

Into a 100 mL round-bottom flask equipped with a magnetic stir bar was added tert-butyl 4-(5-carbamoyl-1,3-thiazol-2-yl)piperazine-1-carboxylate (2.50 g, 8.00 mmol) and THF (50 mL). The suspension was treated with triethylamine (3.35 mL, 24.0 mmol) followed by dropwise addition of TFAA (1.7 mL, 12.0 mmol) over 20 min. The resulting solution was stirred at room temperature for 30 min and then poured into a 250 mL separatory funnel containing saturated aqueous NaHCO₃ (50 mL) and the mixture was extracted with ethyl acetate (3×50 mL). The combined organic layers were washed with brine, dried over MgSO₄, filtered and the solvent evaporated under reduced pressure. Purification by column chromatography through silica gel, eluting with 0% EtOAc in hexanes to 50% EtOAc in hexanes as a gradient, afforded the title compound as a white solid.

### Step 4: tert-Butyl 4-[5-(2H-tetrazol-5-yl)-1,3-thiazol-2-yl]piperazine-1-carboxylate

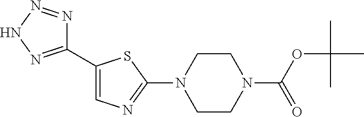

Into a 100 mL pressure flask equipped with a magnetic stir bar was added tert-butyl 4-(5-cyano-1,3-thiazol-2-yl)piperazine-1-carboxylate (1.50 g, 5.10 mmol), sodium azide (1.65 g, 25.5 mmol), ammonium chloride (1.36 g, 25.5 mmol) and dioxane (25 mL). The vial was sealed and the reaction mixture was stirred at 110° C. in an oil bath for 16 h. The cooled reaction mixture was diluted with water (25 mL) and acidified to pH 3 with 1 N aqueous HCl solution. The resulting suspension was filtered through filter paper on a Hirsch funnel, washing with water (5 mL). The grey solid was dried under vacuum for 6 h to afford the title compound.

### Step 5: tent-Butyl 4-{5-[2-(2-ethoxy-2-oxoethyl)-2H-tetrazol-5-yl]-1,3-thiazol-2-yl}piperazine-1-carboxylate

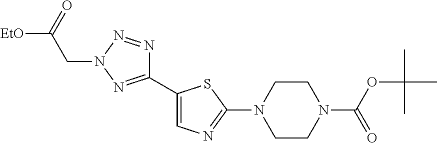

Into a 75 mL sealable pressure flask equipped with a magnetic stir bar was added tert-butyl 4-[5-(2H-tetrazol-5-yl)-1,3-thiazol-2-yl]piperazine-1-carboxylate (1.30 g, 3.85 mmol) in THF (15 mL). The solution was treated with triethylamine (1.1 mL, 7.70 mmol) followed by ethyl bromoacetate (1.3 mL, 11.6 mmol). The vial was sealed and heated to 80° C. in an oil bath for 1 h. The mixture was cooled to room temperature and poured into a 250 mL separatory funnel containing water (100 mL) and extracted with ethyl acetate (3×30 mL). The combined organic layers were washed with brine, dried over MgSO₄, filtered and the solvent was evaporated under reduced pressure. Purification by column chromatography through silica gel, eluting with 10% EtOAc in hexanes to 75% EtOAc in hexanes as a gradient, afforded the desired product as a single regioisomer.

### Step 6: Ethyl {5-[2-(piperazin-1-yl)-1,3-thiazol-5-yl]-2H-tetrazol-2-yl}acetate Hydrochloride

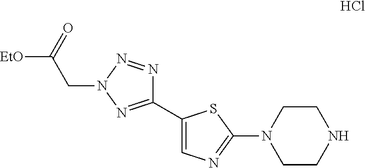

Into a 100 mL round-bottom flask equipped with a magnetic stir bar was added tert-butyl 4-{5-[2-(2-ethoxy-2-oxoethyl)-2H-tetrazol-5-yl]-1,3-thiazol-2-yl}piperazine-1-carboxylate (1.30 g, 3.07 mmol) and 4.0 M HCl in dioxane (14.8 mL, 59 mmol). The resulting suspension was stirred at room temperature for 3 h. The suspension was filtered through filter paper on a Hirsch funnel, washing with diethyl ether (5 mL) and the resulting white solid was dried under vacuum for 2 h. MS (ESI, Q⁺) m/z 324 (M+1).

### Intermediate 8

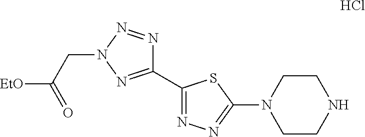

### Ethyl {5-[5-(piperazin-1-yl)-1,3,4-thiadiazol-2-yl]-2H-tetrazol-2-yl}acetate hydrochloride

### Step 1: 5-Bromo-1,3,4-thiadiazol-2-amine

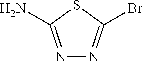

Into a 250 mL round-bottom flask equipped with a magnetic stir bar was added 1,3,4-thiadiazol-2-amine (10.0 g, 99 mmol) and sodium acetate (8.92 g, 109 mmol) in concentrated acetic acid (57 mL). The suspension was treated with dropwise addition of bromine (5.60 mL, 109 mmol) and the yellow-orange suspension was stirred at room temperature for 3 h. The reaction mixture was diluted with water (100 mL) and filtered through filter paper on a Hirsch funnel, washing with water to give a light beige solid.

### Step 2: 5-Bromo-1,3,4-thiadiazole-2-carbonitrile

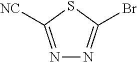

Into a 100 mL round-bottom flask equipped with a magnetic stir bar was added 5-bromo-1,3,4-thiadiazol-2-amine (6.00 g, 33.3 mmol) and copper(I) cyanide (6.57 g, 73.3 mmol) in MeCN (111 mL). The suspension was cooled to 0° C. and tert-butyl nitrite (8.30 mL, 70.0 mmol) was added dropwise over 0.5 h. After stirring at room temperature for an additional 1 h, the reaction mixture was filtered through a pad of silica gel on a sintered glass funnel, washing with ethyl acetate (100 mL). The filtrate was concentrated under reduced pressure and purified by column chromatography through silica gel, eluting with 0% EtOAc in hexanes to 50% EtOAc in hexanes as a gradient. The desired product was obtained as an off-white solid.

### Step 3: tert-Butyl 4-(5-cyano-1,3,4-thiadiazol-2-yl)piperazine-1-carboxylate

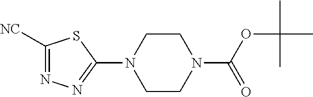

Into a 50 mL round-bottom flask equipped with a magnetic stir bar was added 5-bromo-1,3,4-thiadiazole-2-carbonitrile (1.00 g, 5.26 mmol) and dioxane (30 mL). The solution was treated with tert-butyl piperazine-1-carboxylate (1.08 g, 5.79 mmol) followed by DIPEA (2.3 mL, 13.2 mmol) and the reaction mixture was stirred for 1 h at room temperature. The mixture was poured into a 250 mL separatory funnel containing saturated aqueous NH₄Cl (100 mL) and extracted with ethyl acetate (3×30 mL). The combined organic layers were washed with brine, dried over MgSO₄, filtered and the solvent was evaporated under reduced pressure. Purification by column chromatography through silica gel, eluting with 0% EtOAc in hexanes to 50% EtOAc in hexanes as a gradient, afforded the desired product as a yellow solid.

### Step 4: tert-Butyl 4-[5-(2H-tetrazol-5-yl)-1,3,4-thiadiazol-2-yl]piperazine-1-carboxylate

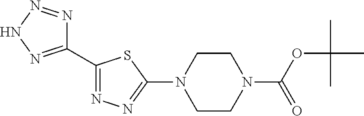

Into a 100 mL pressure flask equipped with a magnetic stir bar was added tert-butyl 4-(5-cyano-1,3,4-thiadiazol-2-yl)piperazine-1-carboxylate (1.40 g, 4.74 mmol), sodium azide (1.54 g, 23.7 mmol), ammonium chloride (1.27 g, 23.7 mmol) and dioxane (25 mL). The vial was sealed and the reaction mixture was stirred at 110° C. in an oil bath for 16 h. The reaction mixture was cooled to room temperature and diluted with water (25 mL). The mixture was acidified to pH 3 with 1 N aqueous HCl solution and stirred for 0.5 h. The resulting suspension was filtered through filter paper on a Hirsch funnel under vacuum, washing with water (5 mL). The resulting beige cake was dried under vacuum for 6 h.

MS (ESI, Q⁺) m/z 337 (M+1).

### Step 5: tert-Butyl 4-{5-[2-(2-ethoxy-2-oxoethyl)-2H-tetrazol-5-yl]-1,3,4-thiadiazol-2-yl}piperazine-1-carboxylate

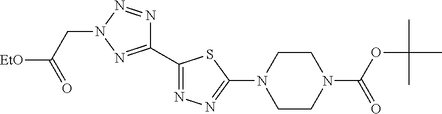

Into a 10 mL sealable pressure flask equipped with a magnetic stir bar was added tert-butyl 4-[5-(2H-tetrazol-5-yl)-1,3,4-thiadiazol-2-yl]piperazine-1-carboxylate (300 mg, 0.89 mmol) in THF (3.0 mL). The solution was treated with triethylamine (0.25 mL, 1.77 mmol) followed by ethyl bromoacetate (0.30 mL, 2.66 mmol). The vial was sealed and heated to 80° C. in an oil bath for 1 h. The reaction mixture was cooled to room temperature and diluted with water (5 mL). The mixture was poured into a phase separation cartridge and extracted with dichloromethane (2×5 mL) and the combined organics were concentrated under reduced pressure. Purification by column chromatography through silica gel, eluting with 10% EtOAc in hexanes to 75% EtOAc in hexanes as a gradient, afforded the desired regioisomeric product.

### Step 6: Ethyl {5-[5-(piperazin-1-yl)-1,3,4-thiadiazol-2-yl]-2H-tetrazol-2-yl}acetate Hydrochloride

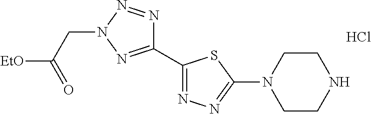

Into a 50 mL round-bottom flask equipped with a magnetic stir bar was added tert-butyl 4-{5-[2-(2-ethoxy-2-oxoethyl)-2H-tetrazol-5-yl]-1,3,4-thiadiazol-2-yl}piperazine-1-carboxylate (200 mg, 0.47 mmol) and 4.0 M HCl in dioxane (2.4 mL, 9.5 mmol). The resulting suspension was stirred at room temperature for 3 h, filtered through filter paper on a Hirsch funnel under vacuum and washed with diethyl ether (3 mL). The title compound was obtained as a white solid. MS (ESI, Q⁺) m/z 335 (M+1).

### Intermediate 9

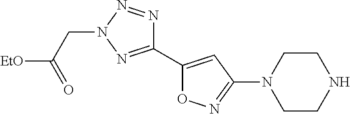

### Ethyl [5-(3-piperazin-1-ylisoxazol-5-yl)-2H-tetrazol-2-yl]acetate

### Step 1: Benzyl 4-[5-(aminocarbonyl)-4,5-dihydroisoxazol-3-yl]piperazine-1-carboxylate

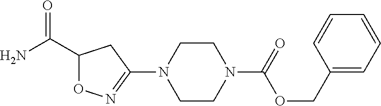

A mixture of 3-bromo-4,5-dihydroisoxazole-5-carboxamide (Intermediate 3, 6.0 g, 31.1 mmol), benzyl piperazine-1-carboxylate (1.4 g, 6.22 mmol) and DIPEA (2.3 mL, 12.95 mmol) in ethanol (60 mL) was heated at 100° C. for 18 h. The solvent was evaporated, the mixture diluted with 5% aqueous citric acid solution (50 mL), and the suspension was filtered through filter paper on a Hirsch funnel, washing the resulting solid with water and Et₂O. The solid was dried under high vacuum to afford the title product. MS (ESI, Q⁺) m/z 333 (M+1).

### Step 2: Benzyl 4-[5-(aminocarbonyl)isoxazol-3-yl]piperazine-1-carboxylate

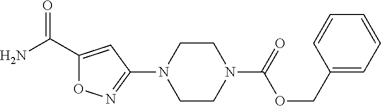

To a stirred suspension of benzyl 4-[5-(aminocarbonyl)-4,5-dihydroisoxazol-3-yl]piperazine-1-carboxylate (4.5 g, 13.5 mmol) and sodium acetate (2.78 g, 33.8 mmol) in toluene (45 mL) was added iodine (4.47 g, 17.6 mmol). The mixture was heated at reflux temperature for 12 h. After cooling, the mixture was diluted with saturated aqueous Na₂S₂O₃ solution (10 mL). The solvents were evaporated under reduced pressure and the mixture was triturated with Et₂O (25 mL). The resulting suspension was filtered through filter paper on a Hirsch funnel, washing with water followed by Et₂O. The title compound was obtained as a solid. MS (ESI, Q⁺) m/z 331 (M+1).

### Step 3: Benzyl 4-(5-cyanoisoxazol-3-yl)piperazine-1-carboxylate

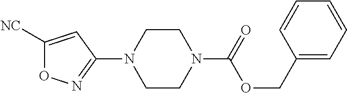

To a solution of benzyl 4-[5-(aminocarbonyl)isoxazol-3-yl]piperazine-1-carboxylate (3.8 g, 11.5 mmol) and triethylamine (4.0 mL, 29 mmol) in THF (38 mL) was added TFAA (1.95 mL, 13.8 mmol) at 0° C. The reaction mixture was warmed to room temperature and stirred for 0.5 h. The solvent was evaporated under reduced pressure and the mixture was purified by column chromatography through silica gel, eluting with 10% EtOAc in hexanes to 40% EtOAc in hexanes as a gradient, to afford the title compound.

MS (ESI, Q⁺) m/z 335 (M+Na).

### Step 4: Benzyl 4-[5-(1H-tetrazol-5-yDisoxazol-3-yl]piperazine-1-carboxylate

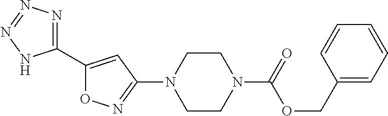

A mixture of benzyl 4-(5-cyanoisoxazol-3-yl)piperazine-1-carboxylate (3.1 g, 9.93 mmol), sodium azide (1.94 g, 29.8 mmol) and ammonium chloride (2.12 g, 39.7 mmol) in DMF (20 mL) was heated at 100° C. for 1 h. The mixture was cooled to rt, diluted with aqueous 2 M HCl solution (50 mL) and hexanes (25 mL). The mixture was filtered through filter paper on a Hirsch funnel, washing with water followed by hexanes. The solid was dried under high vacuum to afford the title compound. MS (ESI, Q⁺) m/z 356 (M+1).

### Step 5: Benzyl 4-{5-[2-(2-ethoxy-2-oxoethyl)-2H-tetrazol-5-yl] isoxazol-3-yl}piperazine-1-carboxylate

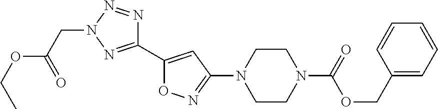

A mixture of benzyl 4-[5-(1H-tetrazol-5-yl)isoxazol-3-yl]piperazine-1-carboxylate (3.2 g, 9.0 mmol), triethylamine (2.51 mL, 18.0 mmol) and ethyl bromoacetate (1.5 mL, 13.5 mmol) in THF (30 mL) was heated at 80° C. for 1 h. The solvent was evaporated under reduced pressure and the mixture diluted with water (25 mL) and Et₂O (10 mL). The resulting suspension was filtered through filter paper on a Hirsch funnel, washing with water and Et₂O. The solid was dried under high vacuum to afford the title product as a single regioisomer. The more polar isomer, benzyl 4-{5-[1-(2-ethoxy-2-oxoethyl)-1H-tetrazol-5-yl]isoxazol-3-yl}piperazine-1-carboxylate, was present in the filtrate.

MS (ESI, Q⁺) m/z 442 (M+1).

### Step 6: Ethyl [5-(3-piperazin-1-ylisoxazol-5-yl)-2H-tetrazol-2-yl]acetate

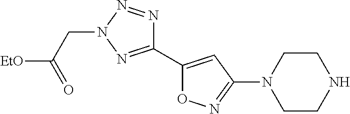

A mixture of benzyl 4-{5-[2-(2-ethoxy-2-oxoethyl)-2H-tetrazol-5-yl]isoxazol-3-yl}piperazine-1-carboxylate (3.2 g, 7.25 mmol) and Pd/C (0.077 g, 0.725 mmol) in THF (24 mL) and ethanol (12 mL) was hydrogenated at rt for 3 h. The mixture was filtered through celite and the solvent was evaporated to afford the title product as a solid which was used without purification.

¹H NMR (Acetone-d₆, 500 MHz): δ 6.94 (1H, s), 5.82 (2H, s), 4.30 (2H, q, J=7.0 Hz), 3.31 (4H, t, J=5.0 Hz), 2.93 (4H, t, J=5.0 Hz), 1.30 (3H, t, J=7.0 Hz). MS (ESI, Q⁺) m/z 308 (M+1).

### Intermediate 10

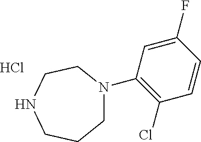

### 1-(2-Chloro-5-fluorophenyl)-1,4-diazepane Hydrochloride

### Step 1: tert-Butyl 4-(2-chloro-5-fluorophenyl)-1,4-diazepane-1-carboxylate

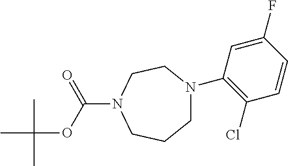

Into a 25 mL pressure vial equipped with a magnetic stir bar was added racemic BINAP (0.622 g, 1.00 mmol), palladium acetate (0.112 g, 0.50 mmol) and sodium tert-butoxide (1.152 g, 12.0 mmol). The flask was evacuated under vacuum (1 mm Hg) and backfilled with nitrogen (repeated 3 times). To the flask was added toluene (5 ml), 2-chloro-5-fluoro-iodobenzene (2.82 g, 11.0 mmol) and tert-butyl 1,4-diazepane-1-carboxylate (2.00 g, 10.0 mmol). The dark suspension was degassed with a steady flow of nitrogen for 10 min and then heated to 120° C. for 16 h. The resulting dark brown suspension was cooled to room temperature and filtered through a pad of silica gel on a sintered glass funnel, washing with ethyl acetate (200 mL). The filtrate was concentrated and purified by column chromatography through silica gel, eluting with 0% EtOAc in hexanes to 50% EtOAc in hexanes as a gradient. The title compound was obtained as a yellow oil. MS (ESI, Q⁺) m/z 229 (M+1-tert-butoxycarbonyl).

### Step 2: 1-(2-Chloro-5-fluorophenyl)-1,4-diazepane Hydrochloride

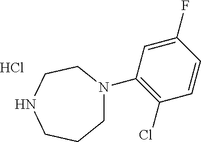

Into a 100 mL round-bottom flask equipped with a magnetic stir bar was added tert-butyl 4-(2-chloro-5-fluorophenyl)-1,4-diazepane-1-carboxylate (1.079 g, 3.28 mmol) and 4.0 M HCl in dioxane (8.20 ml, 32.8 mmol). The resulting mixture was stirred at room temperature for 1 h. The suspension was diluted with diethyl ether (5 mL) and filtered through filter paper, washing with diethyl ether (5 mL). The resulting light yellow solid was dried on the vacuum pump for 1 h.

### Intermediate 11

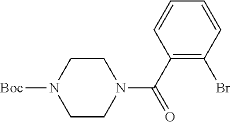

### tert-Butyl 4-[(2-bromophenyl)carbonyl]piperidine-1-carboxylate

### Step 1: tert-Butyl 4-{[2-(4,4,5,5-tetramethyl-1,3,2-dioxaborolan-2-yl)phenyl]carbonyl}piperidine-1-carboxylate

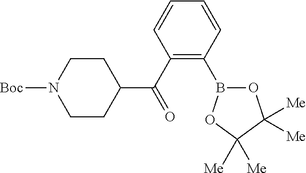

Into a 100 mL round-bottom flask equipped with a magnetic stir bar was added bis(pinacolato)diboron (1.18 g, 4.63 mmol), tert-butyl 4-[(2-chlorophenyl)carbonyl]piperidine-1-carboxylate (1.25 g, 3.86 mmol), Pd₂ dba₃ (0.21 g, 0.23 mmol), tricyclohexylphosphine (0.26 g, 0.93 mmol) and potassium acetate (1.14 g, 11.6 mmol). The flask was evacuated under vacuum (1 mm Hg) and backfilled with N₂ (repeated 3 times). The solids were diluted with 1,4-dioxane (25 mL) and degassed for 10 minutes before being heated to 80° C. for 3 days. The cooled reaction mixture was diluted with diethyl ether (50 mL) and filtered through a pad of celite on a sintered glass funnel, washing with diethyl ether (2×25 mL). The filtrate was concentrated to an oil and purified by column chromatography through silica gel, eluting with 0% EtOAc in hexanes to 40% EtOAc in hexanes as a gradient to give the title compounds as a yellow oil.

### Step 2: tert-Butyl 4-[(2-bromophenyl)carbonyl]piperidine-1-carboxylate

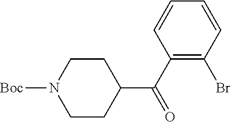

Into a 50 mL round-bottom flask equipped with a magnetic stir bar was added tert-butyl 4-{[2-(4,4,5,5-tetramethyl-1,3,2-dioxaborolan-2-yl)phenyl]carbonyl}piperidine-1-carboxylate (900 mg, 2.167 mmol) and methanol (10 mL). To this was added a solution of copper(II) bromide (1.40 g, 6.50 mmol) in water (10 mL) and the mixture was heated to reflux for 2 h. The cooled reaction mixture was filtered through a pad of celite on a sintered glass funnel, washing with ethyl acetate (100 mL). The filtrate was concentrated and diluted with ethyl acetate (50 mL) and water (50 mL) poured into a 250 mL separatory funnel. The aqueous layer was extracted with ethyl acetate (50 mL), and the combined organic layers were set aside. The aqueous layer was basified to pH=10 with 10 M aqueous NaOH solution (0.650 ml, 6.50 mmol) followed by the addition of di-tert-butyl dicarbonate (2.00 ml, 8.67 mmol). The reaction mixture was stirred at room temperature for 4 h. The mixture was poured into a 250 mL separatory funnel and extracted with ethyl acetate (3×30 mL). The combined organic layers (including the organic layer from the previous work-up above) were washed with brine, dried over MgSO₄, filtered and the solvent was evaporated under reduced pressure. Purification by column chromatography through silica gel, eluting with 0% EtOAc in hexanes to 50% EtOAc in hexanes as a gradient afforded the title compound as a light yellow oil. MS (ESI, Q⁺) m/z 390, 392 (M+Na).

### Intermediate 12

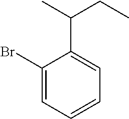

### 1-Bromo-2-(butan-2-yl)benzene

Into a 50 mL round-bottom flask cooled to 0° C. equipped with a magnetic stir bar was dissolved 2-sec-butylaniline (5.0 g, 33.5 mmol) in hydrobromic acid (9.5 mL). A solution of sodium nitrite (2.3 g, 33.5 mmol) in water (4.2 mL) was added drop wise to the first solution. The resulting solution was added to a refluxing copper(I) bromide (2.6 g, 18.4 mmol) solution in hydrobromic acid (2.3 mL). The mixture was cooled to room temperature and then poured into a 500 mL separatory funnel and extracted with ethyl acetate (3×100 mL). The combined organic layers were washed with brine, dried over MgSO₄, filtered and the solvent was evaporated under reduced pressure. Purification by column chromatography through silica gel, eluting with 0% EtOAc in hexanes to 15% EtOAc in hexanes as a gradient afforded the title compound.

### Intermediate 13

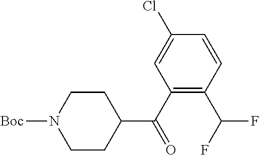

### tert-Butyl-4-{[5-chloro-2-(difluoromethyl)phenyl]carbonyl}piperidine-1-carboxylate

### Step 1: (2-Bromo-4-chlorophenyl)methanol

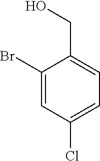

Into a 500 mL round-bottom flask equipped with a magnetic stir bar was dissolved 2-bromo-4-chlorophenylbenzoic acid (10.0 g, 42.5 mmol) in THF (42.5 mL). It was stirred at rt for 18 h. The mixture was quenched with 10% aqueous HCl and it was poured into a 1000 mL separatory funnel and extracted with ethyl acetate (3×200 mL). The combined organic layers were washed with brine, dried over MgSO₄, filtered and the solvent was evaporated under reduced pressure to afford the title compound.

### Step 2: [(2-bromo-4-chlorobenzyl)oxy](tripropan-2-yl)silane

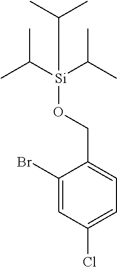

Into a microwave vial equipped with a magnetic stir bar was mixed (2-bromo-4-chlorophenyl)methanol (5.0 g, 22.6 mmol) with chlorotriisopropylsilane (6.5 g, 33.9 mmol) and imidazole (6.2 g, 90.0 mmol). The tube was sealed and it was heated in the microwave oven at 110° C. for 10 min. The mixture was quenched with 10% aqueous HCl and it was poured into a 500 mL separatory funnel and extracted with ethyl acetate (3×100 mL). The combined organic layers were washed with brine, dried over MgSO₄, filtered and the solvent was evaporated under reduced pressure. Purification by column chromatography through silica gel, eluting with 0% EtOAc in hexanes to 15% EtOAc in hexanes as a gradient afforded the title compound.

### Step 3: tert-Butyl-4-[(5-chloro-2-{[(tripropan-2-ylsilyl)oxy]methyl}phenyl)carbonyl]piperidine-1-carboxylate

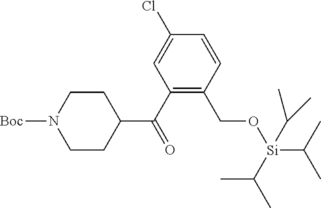

tert-Butyl-4-[(5-chloro-2-{[(tripropan-2-ylsilyl)oxy]methyl}phenyl)carbonyl]piperidine-1-carboxylate was obtained following step 3 in example 16. Purification by column chromatography through silica gel, eluting with 0% EtOAc in hexanes to 20% EtOAc in hexanes as a gradient afforded the title compound.

### Step 4: tert-Butyl-4-{[5-chloro-2-(hydroxymethyl)phenyl]carbonyl}piperidine-1-carboxylate

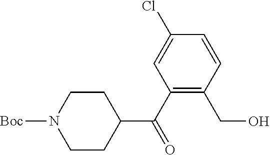

Into a 400 mL Nalgene beaker equipped with a magnetic stir bar was dissolved tert-butyl-4-[(5-chloro-2-{[(tripropan-2-ylsilyl)oxy]methyl}phenyl)carbonyl]piperidine-1-carboxylate (7.0 g, 13.7 mmol) in THF (46 mL) and it was cooled to −78° C. HF.pyridine (12 mL, 120 mmol) was added and it was warmed up to rt. TBAF (1M, 10 mL, 10 mmol) was added. After 1 h, it was carefully quenched over sat. aq. NaHCO₃ (400 mL). It was then transferred into a 1 L separatory funnel and extracted with ethyl acetate (3×200 mL). The combined organic layers were washed with brine, dried over MgSO₄, filtered and the solvent was evaporated under reduced pressure. Purification by column chromatography through silica gel, eluting with 0% EtOAc in hexanes to 30% EtOAc in hexanes as a gradient afforded the title compound.

### Step 5: tert-Butyl-4-[(5-chloro-2-formylphenyl)carbonyl]piperidine-1-carboxylate

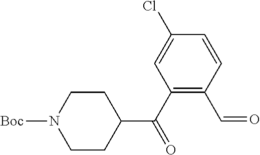

Into a 100 mL round-bottom flask equipped with a magnetic stir bar was dissolved tert-butyl-4-{[5-chloro-2-(hydroxymethyl)phenyl]carbonyl}piperidine-1-carboxylate (2.4 g, 6.8 mmol) in CH₂Cl₂ (22 mL) and it was cooled to 0° C. Dess-Martin periodinane (3.2 g, 7.5 mmol) was added. The ice bath was removed. After 2 h, the reaction mixture was transferred into a 250 mL separatory funnel containing 100 mL of 1N NaOH and extracted with CH₂Cl₂ (3×50 mL). The combined organic layers were washed with brine, dried over MgSO₄, filtered and the solvent was evaporated under reduced pressure. Purification by column chromatography through silica gel, eluting with 20% EtOAc in hexanes to 50% EtOAc in hexanes as a gradient afforded the title compound.

### Step 6: tert-Butyl-4-{[5-chloro-2-(difluoromethyl)phenyl]carbonyl}piperidine-1-carboxylate

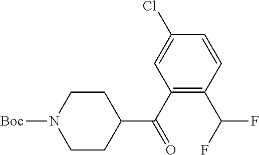

Into a 100 mL round-bottom flask equipped with a magnetic stir bar was dissolved tert-butyl-4-[(5-chloro-2-formylphenyl)carbonyl]piperidine-1-carboxylate (1.2 g, 3.4 mmol) in CH₂Cl₂ (11.5 mL) and it was cooled to −78° C. DAST (1.2 g, 7.6 mmol) was added and it was warmed up to rt. After 1 h, it was transferred into a 125 mL separatory funnel containing 40 mL of sat aq. NaHCO₃ and extracted with ethyl acetate (3×25 mL). The combined organic layers were washed with brine, dried over MgSO₄, filtered and the solvent was evaporated under reduced pressure. Purification by column chromatography through silica gel, eluting with 15% EtOAc in hexanes to 50% EtOAc in hexanes as a gradient and by reverse phase HPLC afforded the title compound.

### Intermediate 14

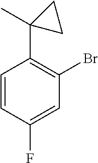

### 2-Bromo-4-fluoro-1-(1-methylcyclopropyl)benzene

### Step 1: 2-Bromo-4-fluoro-1-(prop-1-en-2-yl)benzene

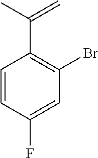

Into a 500 mL round-bottom flask equipped with a magnetic stir bar was dissolved methyl triphenylphosphonium bromide (24.7 g, 69.1 mmol) in THF and it was cooled to 0° C. n-BuLi (27.6 mL, 2.5 M in hexanes, 69.1 mmol) was added. After 20 min, 1-(2-bromo-4-fluorophenyl)ethanone (10 g, 46.1 mmol) in 5 mL of THF was added to the reaction mixture. It was stirred at rt for 18 h. It was transferred into a 1000 mL separatory funnel containing 300 mL of 10% aq HCl and extracted with ethyl acetate (3×200 mL). The combined organic layers were washed with brine, dried over MgSO₄, filtered and the solvent was evaporated under reduced pressure. Purification by column chromatography through silica gel, eluting with 0% EtOAc in hexanes to 15% EtOAc in hexanes as a gradient afforded the title compound.

### Step 2: 2-Bromo-4-fluoro-1-(1-methylcyclopropyl)benzene

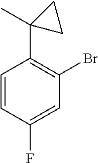

Into a 100 mL flame-dried round-bottom flask equipped with a magnetic stir bar was dissolved diethylzinc (1.1 g, 9.3 mmol) in CH₂Cl₂ (10 mL) and it was cooled to 0° C. Trifluoroacetic acid. (1.1 g, 9.3 mmol) in 5 mL of CH₂Cl₂ was added very slowly. After 20 min, diiodomethane (2.5 g, 9.3 mmol) in 5 mL of CH₂Cl₂ was added. After 20 min, 2-bromo-4-fluoro-1-(prop-1-en-2-yl)benzene (1.0 g, 4.7 mmol) in 5 mL of CH₂Cl₂ was added. The reaction mixture was allowed to warm up to rt and it was stirred for 40 min. It was then transferred into a 250 mL separatory funnel containing 75 mL of 10% aq HCl and extracted with ethyl acetate (2×70 mL). The combined organic layers were washed with brine, dried over MgSO₄, filtered and the solvent was evaporated under reduced pressure. Purification by column chromatography through silica gel, eluting with 100% hexanes afforded the title compound.

### Intermediate 15

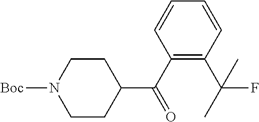

### tert-Butyl-4-{[2-(2-fluoropropan-2-yl)phenyl]carbonyl}piperidine-1-carboxylate

### Step 1: 1-Bromo-2-(2-fluoropropan-2-yl)benzene

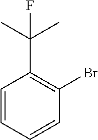

Into a 100 mL round-bottom flask equipped with a magnetic stir bar was dissolved 2-(2-bromophenyl)propan-2-ol (1.0 g, 4.7 mmol) in CH₂Cl₂ (15 mL) and it was cooled to −78° C. DAST (1.1 g, 7.0 mmol) was added and the reaction was monitored by TLC. After disappearance of the starting material, the reaction mixture was quenched over 50 mL of a sat aq. NaHCO₃ solution in a beaker. It was then transferred into a 250 mL separatory funnel and extracted with ethyl acetate (2×70 mL). Combined organic layers were washed with brine, dried over MgSO₄, filtered and the solvent was evaporated under reduced pressure. Purification by column chromatography through silica gel, eluting with 100% hexanes afforded the title compound.

### Step 2: tert-Butyl-4-{[2-(2-fluoropropan-2-yl)phenyl](hydroxy)methyl}piperidine-1-carboxylate

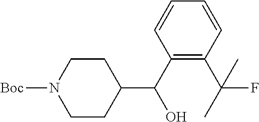

Into a 100 mL flame-dried round-bottom flask equipped with a magnetic stir bar was dissolved 1-bromo-2-(2-fluoropropan-2-yl)benzene (952 mg, 4.4 mmol) in THF (11 mL) and it was cooled to −78° C. t-BuLi (5.2 mL, 8.8 mmol, 1.7 M in pentane) was added drop wise. Then, tert-butyl-4-formylpiperidine-1-carboxylate (850 mg, 4.0 mmol) in THF (2.0 mL) was added. After 15 min, the reaction mixture was warmed up to rt. After 1.5 h, it was transferred into a 125 mL separatory funnel containing 50 mL of 10% aq HCl and extracted with ethyl acetate (2×40 mL). The combined organic layers were washed with brine, dried over MgSO₄, filtered and the solvent was evaporated under reduced pressure. Purification by column chromatography through silica gel, eluting with 0% EtOAc in hexanes to 60% EtOAc in hexanes as a gradient afforded the title compound.

### Step 3: tert-Butyl-4-{[2-(2-fluoropropan-2-yl)phenyl]carbonyl}piperidine-1-carboxylate

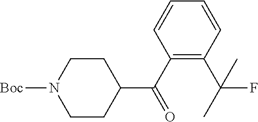

Into a 100 mL round-bottom flask equipped with a magnetic stir bar was dissolved tert-butyl-4-{[2-(2-fluoropropan-2-yl)phenyl](hydroxy)methyl}piperidine-1-carboxylate (620 mg, 1.8 mmol) in CH₂Cl₂ (8.8 mL) and Dess-Martin periodinane (748 mg, 1.8 mmol) was added. It was stirred at rt for 16 h. It was transferred into a 125 mL separatory funnel containing 50 mL of 1N NaOH and extracted with diethyl ether (2×40 mL). The combined organic layers were washed with brine, dried over MgSO₄, filtered and the solvent was evaporated under reduced pressure. Purification by column chromatography through silica gel, eluting with 10% EtOAc in hexanes to 60% EtOAc in hexanes as a gradient afforded the title compound.

### Intermediate 16

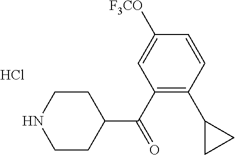

### [2-Cyclopropyl-5-(trifluoromethoxy)phenyl](piperidin-4-yl)methanone Hydrochloride

### Step 1: tert-Butyl-4-{[2-chloro-5-(trifluoromethoxy)phenyl]carbonyl}piperidine-1-carboxylate

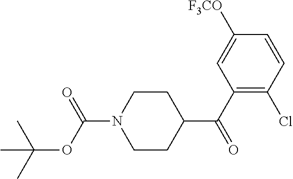

To a −78° C. solution of 2-bromo-1-chloro-4-(trifluoromethoxy)benzene (2.95 g, 10.7 mmol) in THF (55 mL) was slowly added tert-butyllithium (1.7 M in pentanes, 12.6 mL, 21.4 mmol). After stirring at −78° C. for a few minutes, a solution of tert-butyl-4-(methoxy(methyl)carbamoyl)piperidine-1-carboxylate (2.65 g, 9.73 mmol) in THF (2 mL) was added to the reaction mixture. At the end of the addition, the cold bath was removed and the reaction mixture was warmed to room temperature and stirred at this temperature for 1 h. The reaction mixture was re-cooled to −10° C. and quenched with a saturated solution of ammonium chloride. The mixture was poured into a separatory funnel and extracted with ethyl acetate. The combined organic layers were washed with brine, dried over MgSO₄, filtered and the solvent removed under reduced pressure. Purification by column chromatography through silica gel, eluting with 2% EtOAc in hexanes to 30% EtOAc in hexanes as a gradient, afforded the title compound.

### Step 2: tert-Butyl-4-{[2-cyclopropyl-5-(trifluoromethoxy)phenyl]carbonyl}piperidine-1-carboxylate

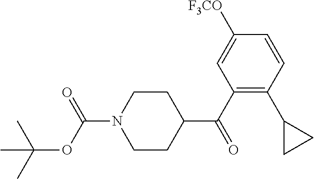

Into a 100 mL round bottom flask equipped with a stir bar was added tert-butyl-4-{[2-chloro-5-(trifluoromethoxy)phenyl]carbonyl}piperidine-1-carboxylate (998 mg, 2.5 mmol), cyclopropylboronic acid (631 mg, 7.3 mmol), palladium (II) acetate (55 mg, 0.25 mmol), potassium phosphate tribasic (6.2 g, 29.4 mmol), toluene (15 mL) and water (1.5 mL). The reaction mixture was degassed for 10 minutes, by passing nitrogen through a needle immersed in the reaction mixture. Then, tricyclohexylphosphine (1M in THF, 0.48 mL, 0.48 mmol) was added and the reaction mixture was heated at 80° C. under nitrogen atmosphere for 24 h, after which it was quenched with water. The reaction mixture was filtered through celite. The filtrate was poured into a separatory funnel and extracted with ethyl acetate. The combined organic layers were washed with brine, dried over Na₂SO₄, filtered and the solvent removed under reduced pressure. Purification by column chromatography through silica gel, eluting with 0% EtOAc in hexanes to 40% EtOAc in hexanes as a gradient, afforded the title compound as a colorless oil. MS (ESI, Q⁺) m/z 436 (M+Na).

### Step 3: [2-Cyclopropyl-5-(trifluoromethoxy)phenyl](piperidin-4-yl)methanone Hydrochloride

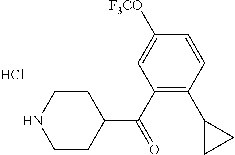

Into a 25 mL round bottom flask equipped with a magnetic stir bar was added a solution of tert-butyl-4-{[2-cyclopropyl-5-(trifluoromethoxy)phenyl]carbonyl}piperidine-1-carboxylate (0.83 g, 2.0 mmol) in dioxane (10 mL). To this was added 4 M HCl in dioxane (5.04 mL, 20.1 mmol), and the reaction mixture was stirred at room temperature for 17 h. The solvent was evaporated under reduced pressure, and the resulting material triturated in diethyl ether (5 mL) for 30 min. The solid was collected by filtration and dried under vacuum. The title compound was obtained as a white solid. MS (ESI, Q⁺) m/z 314 (M+H).

### Intermediate 17

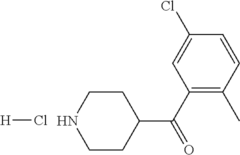

### (5-Chloro-2-methylphenyl)(piperidin-4-yl)methanone Hydrochloride

### Step 1: tert-Butyl 4-(5-chloro-2-methylbenzoyl)piperidine-1-carboxylate

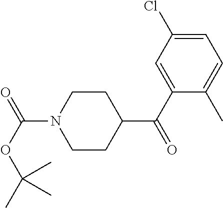

To a solution of n-butylmagnesium chloride (1.5 mL, 3.0 mmol) in THF (15 mL) stirred at −78° C., n-BuLi (3.75 mL, 6.0 mmol) was added drop wise followed by the addition of 2-bromo-4-chloro-1-methylbenzene (2.00 mL, 15.0 mmol) drop wise. The mixture was stirred at 30° C. for 1 h. Then 1-Boc-4-(methoxy-methyl-carbamoyl)piperidine (1.36 g, 5.0 mmol) was added and the mixture was stirred at rt overnight. The reaction was worked up by the addition of aqueous citric acid, extracted with ethyl acetate, dried over Na₂SO₄, and evaporated. The residue was purified by combiflash (0-20% EtOAc/hexanes) to afford the desired product tert-butyl 4-(5-chloro-2-methylbenzoyl)piperidine-1-carboxylate as clear oil. ¹H NMR (500 MHz, DMSO-d₆): δ 7.75 (s, 1H), 7.48 (d, 1H), 7.34 (d, 1H), 3.93 (d, 2H), 3.25-3.35 (m, 1H), 2.85 (br s, 2H), 2.28 (s, 3H), 1.73 (d, 2H), 1.40 (s, 9H), 1.35 (d, 2H). MS (ESI, Q⁺) m/z 360 (M+Na)

### Step 2: (5-Chloro-2-methylphenyl)(piperidin-4-yl)methanone Hydrochloride

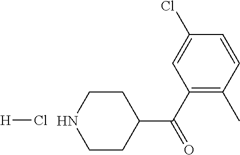

A solution of tert-butyl 4-(5-chloro-2-methylbenzoyl)piperidine-1-carboxylate (1.6 g, 4.7 mmol) in 4 M HCl/1,4-dioxane (20 mL) was stirred at rt for 2 h. Then the reaction mixture was diluted with diethyl ether and filtered to collect the solid, washed with 20 mL ether and dried under vacuum to afford (5-chloro-2-methylphenyl)(piperidin-4-yl)methanone hydrochloride.

¹H NMR (500 MHz, DMSO-d₆): δ 7.80 (s, 1H), 7.52 (d, 1H), 7.37 (d, 1H), 3.55 (t, 1H), 3.23-3.34 (m, 2H), 2.97 (t, 2H), 2.31 (s, 3H), 1.91 (d, 2H), 1.73-1.62 (m, 2H). MS (ESI, Q⁺) m/z 238 (M+1).

### Intermediate 18

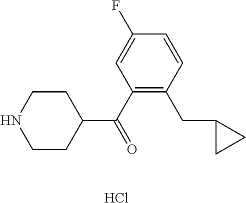

### [2-(Cyclopropylmethyl)-5-fluorophenyl](piperidin-4-yl)methanone Hydrochloride

### Step 1: Dilithium Tetrachloromanganate (Li₂MnCl₄)

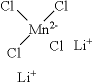

A mixture of 0.25 mol of MnCl₂ and 0.5 mol of LiCl in a 1 L flask was heated to 200° C. under vacuum for 3 h. At the end a heat gun was used to dry the flask wall and stopper. The reaction mixture was then cooled down to room temperature and THF was added to adjust the volume to 1 L. The mixture was stirred at rt overnight and afforded a slightly cloudy solution. The reagent was used in the following reaction by transferring the desired volume from prepared reagent with stirring.

### Step 2: Chloro(1-methylpiperidin-4-yl)magnesium

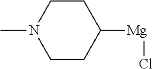

Magnesium turnings (14.5 g, 600 mmol) were mixed with THF (700 mL) at room temperature and 1,2-dibromoethane (2.59 mL, 30.0 mmol) was added drop wise. After the gas evolution was finished, freshly distilled 4-chloro-1-methylpiperidine (80 g, 600 mmol) was added drop wise to magnesium (14.58 g, 600 mmol) at a pace to maintain gentle reflux. The mixture was refluxed for 2 h once the addition was completed and it was then cooled down. This Grignard reagent was used as such in the following reaction.

### Step 3: (2-Bromo-5-fluorophenyl)(1-methylpiperidin-4-yl)methanone

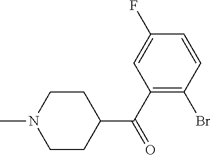

To a suspension of 2-bromo-5-fluorobenzoic acid (5.0 g, 22.8 mmol) in 1,2-dichloroethane (30 mL) stirred at room temperature was added oxalyl chloride (4.0 mL, 45.7 mmol) in one portion. The reaction mixture was stirred at 70° C. for 6 h and the solvent was evaporated under vacuum. The residue was dissolved in THF (60 mL) and cooled to −78° C. Then, chloro(1-methylpiperidin-4-yl)magnesium (28.5 mL, 22.8 mmol) was added drop wise. The mixture was stirred at −78° C. for 10 min and was allowed to warm up to rt. The reaction mixture was cooled in an ice bath, water (200 mL) was added and the mixture was extracted with ethyl acetate (2×100 mL). The combined organic layers were washed with brine, dried (MgSO₄), filtered and the solvent was evaporated under reduced pressure. The residue was purified by column chromatography on silica gel (Isolute Flash Si; 100 g prepacked), eluting with 0-15% CH₂Cl₂/MeOH to give (2-bromo-5-fluorophenyl)(1-methylpiperidin-4-yl)methanone as a yellowish solid. ¹H NMR (400 MHz, DMSO-d₆): δ 7.75 (dd, 1H), 7.53 (dd, 1H), 7.32 (td, 1H), 2.97 (tt, 1H), 2.76 (d, 2H), 2.15 (s, 3H), 1.94-1.84 (m, 2H), 1.76 (d, 2H), 1.60-1.48 (m, 2H). MS (ESI, Q⁺) m/z 300 (M+1).

### Step 4: [2-(Cyclopropylmethyl)-5-fluorophenyl](1-methylpiperidin-4-yl)methanone

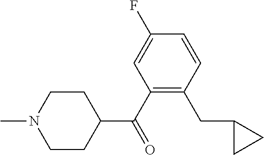

To a flame dried round bottom flask equipped with a stir bar was added Li₂MnCl₄ (46.6 mL, 11.7 mmol). The solution was cooled to −78° C. and cyclopropylmagnesium bromide (15.14 mL, 11.7 mmol) was added drop wise. The reaction mixture was stirred at −46° C. (MeCN and dry ice) for 15 min. It was cooled to −78° C. and a solution of (2-bromo-5-fluorophenyl)(1-methylpiperidin-4-yl)methanone (2.5 g, 8.3 mmol) in THF (20 mL) was added quickly. After stirring for 2 h at −20° C., the reaction mixture was quenched at −20° C. with saturated aqueous NaHCO₃, extracted with EtOAc, then dried (MgSO₄), filtered and evaporated. The residue was purified by column chromatography on silica gel (Isolute Flash Si; 100 g prepacked), eluting with 0-15% CH2Cl2/MeOH to give [2-(cyclopropylmethyl)-5-fluorophenyl](1-methylpiperidin-4-yl)methanone as a yellowish oil. MS (ESI, Q⁺) m/z 276 (M+1).

### Step 5: [2-(Cyclopropylmethyl)-5-fluorophenyl](piperidin-4-yl)methanone Hydrochloride

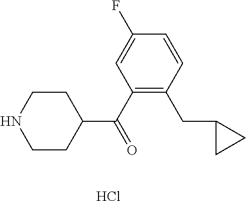

[2-(Cyclopropylmethyl)-5-fluorophenyl](1-methylpiperidin-4-yl)methanone (1.13 g, 4.1 mmol) was dissolved in 1,2-dichloroethane (12 mL) in a reaction tube and 1-chloroethyl chloroformate (0.54 mL, 4.9 mmol) was added at room temperature. Then the reaction was heated to 75° C. for 1 h. The reaction mixture was cooled and 2.5 mL of methanol was added; the reaction was heated to 75° C. for 0.5 h. The mixture was cooled down to rt and the precipitate was collected by filtration and washed with diethyl ether to afford [2-(cyclopropylmethyl)-5-fluorophenyl](piperidin-4-yl)methanone hydrochloride as a white solid. MS (ESI, Q+) m/z 262 (M+1).

### Intermediate 19

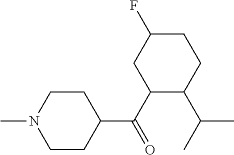

### [5-Fluoro-2-(propan-2-yl)cyclohexyl](1-methylpiperidin-4-yl)methanone

Into a flame dried 25 mL round bottom flask equipped with a magnetic stir bar was added dry zinc chloride* (0.681 g, 5.00 mmol) and anhydrous THF (8.33 mL). To this was then slowly added under nitrogen isopropylmagnesium chloride (2M in THF, 2.50 mL, 5.00 mmol). The resulting white slurry was stirred at 50° C. for 3 h. *Commercially available zinc chloride was carefully melted under a flame and then dried under vacuum for 1 h to yield a white powder. Into a separate 50 mL flame dried round bottom flask equipped with a magnetic stir bar, a solution of (2-bromo-5-fluorocyclohexyl)(1-methylpiperidin-4-yl)methanone (1 g, 3.33 mmol) in anhydrous THF (8.33 ml) was sequentially treated with [1,1′-bis(diphenylphosphino)ferrocene]dichloropalladium(II) (0.122 g, 0.167 mmol) then copper(I) iodide (0.038 g, 0.200 mmol). The alkyl zinc slurry that had been stirring at 50° C. for 3 h was then cannulated slowly into the aryl bromide starting material at rt and the resulting dark brown mixture was left to stir at rt in the dark for 48 h. The reaction was then quenched with 100 mL Na₂CO₃ and extracted with 100 mL EtOAc. The emulsion which resulted was filtered over celite, washing several times with EtOAc and the layers separated. The aqueous phase was further extracted with 100 mL of EtOAc and the combined organic extracts were dried (Na₂SO₄), filtered and concentrated under reduced pressure. Purification by automated flash chromatography on silica gel (0-10% MeOH in CH₂Cl₂) followed by trituration of the resultant oil with ether/hexanes afforded the title compound as an orange oil.

¹H NMR (CDCl₃, 400 MHz): δ 7.32-7.40 (1H, dd), 7.05-7.14 (1H, m), 6.96-7.01 (1H, dd), 3.0-3.1 (1H, m), 2.85-2.95 (2H, m), 2.78-2.84 (1H, m), 2.3 (3H, s), 1.95-2.07 (2H, m), 1.85-1.91 (2H, m), 1.70-1.82 (2H, m), 1.20-1.24 (6H, d).

Ref J Med Chem 2001, vol 44 no. 20 p 3307.

### Intermediate 20

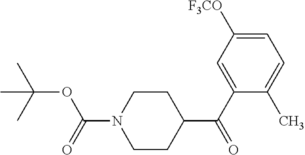

### tert-Butyl-4-{[2-methyl-5-(trifluoromethoxy)phenyl]carbonyl}piperidine-1-carboxylate

To a degassed solution of tert-butyl 4-{[2-chloro-5-(trifluoromethoxy)phenyl]carbonyl}piperidine-1-carboxylate (1.5 g, 3.68 mmol), tetrabutylammonium bromide (1.186 g, 3.68 mmol) and K₂CO₃ (1.017 g, 7.36 mmol) in water (15 mL) and dioxane (15 mL) was added trimethylboroxine (0.693 g, 5.52 mmol) followed by Najera's catalyst (di-μ-chlorobis [5-hydroxy-2-[1-(hydroxyimino)ethyl]phenyl]palladium(II) dimer) (0.021 g, 0.037 mmol). The mixture was refluxed for 20 h under nitrogen. The reaction was further charged with an additional 3×210 mg of Najera's catalyst and 2×3.5 g of trimethylboroxine over a 2 h period. After 4.5 h of reflux, the reaction was cooled and poured into 150 mL water and extracted with EtOAc (3×150 mL). The combined organic extracts were washed with brine (200 mL), dried (Na₂SO₄), filtered and concentrated under reduced pressure. Purification by automated flash chromatography on silica gel (0-50% EtOAc in hexanes) gave a 2:1 inseparable mixture of the title compound and starting material. The impure product was carried on forward without further purification.

Ref Angew Chemie Int Ed. 2002, 41, No. 1 p 179.

The following Examples are provided to illustrate the invention and are not to be construed as limiting the scope of the invention in any manner.

### Example 1

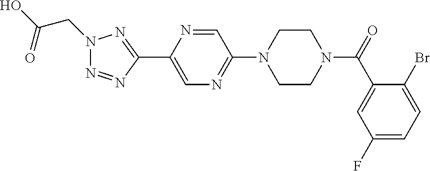

### [5-(5-{4-[(2-Bromo-5-fluorophenyl)carbonyl]piperazin-1-yl}pyrazin-2-yl)-2H-tetrazol-2-yl]acetic Acid

### Step 1: tert-Butyl 4-(pyrazin-2-yl)piperazine-1-carboxylate

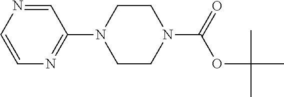

Into a 1 L flask equipped with a condenser and a magnetic stir bar was added 2-chloropyrazine (20.6 g, 180 mmol), tert-butyl piperazine-1-carboxylate (33.5 g, 180 mmol), potassium carbonate (29.8 g, 216 mmol), dioxane (225 mL) and DMF (225 mL). The mixture was heated to 120° C. for 3 days. The mixture was cooled and poured into a 1 L separatory funnel containing brine (600 mL) and extracted with Et₂O (3×200 mL). The combined organic layers were washed with brine, dried over MgSO₄, filtered and concentrated under reduced pressure. Purification by column chromatography through silica gel, eluting with 50:50 hexanes:EtOAc to 20:80 hexanes:EtOAc as a gradient, afforded the title compound as a yellow solid.

### Step 2: tert-Butyl-4-(5-bromopyrazin-2-yl)piperazine-1-carboxylate

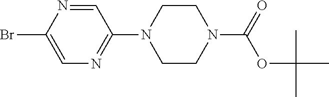

Into a 250 mL flask equipped with a magnetic stir bar was added tert-butyl 4-(pyrazin-2-yl)piperazine-1-carboxylate (5.0 g, 18.9 mmol) and CH₂Cl₂ (95 mL). This solution was cooled to 0° C. and N-bromosuccinimide (4.7 g, 26.5 mmol) was added portion wise over 5 h. The mixture was stirred at 0° C. for 19 h and then poured into a 500 mL separatory funnel containing 200 mL of saturated aqueous NaHCO₃. The mixture was extracted with ethyl acetate (3×100 mL) and the combined organic layers were washed with brine, dried over MgSO₄, filtered and the solvent was evaporated under reduced pressure. Purification by column chromatography through silica gel, eluting with 80:20 hexanes:EtOAc to 50:50 hexanes:EtOAc as a gradient, afforded the title compound as a yellow solid.

¹H NMR (Acetone-d₆, 400 MHz): δ 8.19 (1H, s), 8.10 (1H, s), 3.66-3.61 (4H, m), 3.56 (4H, s), 1.48 (9H, s).

### Step 3: tert-Butyl-4-(5-cyanopyrazin-2-yl)piperazine-1-carboxylate

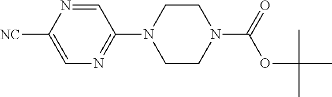

Into a 20 mL microwave tube equipped with a magnetic stir bar was added tert-butyl-4-(5-bromopyrazin-2-yl)piperazine-1-carboxylate (1.8 g, 5.1 mmol) and DMF (10 mL). Nitrogen gas was bubbled into the solution for 2 min and copper(I) cyanide was added (0.91 g, 10.2 mmol). The tube was sealed and heated to 150° C. for 20 min in a microwave reactor. The reaction mixture was filtered through a pad on celite on a sintered glass funnel, and the filtrate was poured into a 250 mL separatory funnel containing 100 mL of saturated aqueous NaHCO₃. The aqueous layer was extracted with ethyl acetate (3×100 mL) and the combined organic layers were washed with brine, dried over MgSO₄, filtered and the solvent was evaporated under reduced pressure. The title compound was used without further purification in Step 4.

### Step 4: tert-Butyl-4-[5-(2H-tetrazol-5-yl)pyrazin-2-yl]piperazine-1-carboxylate

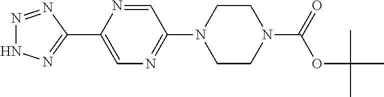

Into a 25 mL pressure tube equipped with a magnetic stir bar was added tert-butyl-4-(5-cyanopyrazin-2-yl)piperazine-1-carboxylate (580 mg, 2.0 mmol), sodium azide (261 mg, 4.0 mmol), ammonium chloride (322 mg, 6.0 mmol) and DMF (10 mL). The tube was sealed and heated to 130° C. for 19 h. The reaction mixture was cooled to room temperature and poured into a 75 mL separatory funnel containing 30 mL of 1 N aqueous NaOH solution. The aqueous layer was washed with diethyl ether (2×30 mL), then acidified to pH 2 with concentrated HCl solution. The resulting precipitate was collected by vacuum filtration to afford the title compound.

### Step 5: tert-Butyl-4-{5-[2-(2-ethoxy-2-oxoethyl)-2H-tetrazol-5-yl]pyrazin-2-yl}piperazine-1-carboxylate

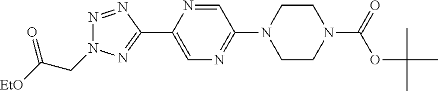

Into a 20 mL pressure vial equipped with a magnetic stir bar was added tert-butyl-4-[5-(2H-tetrazol-5-yl)pyrazin-2-yl]piperazine-1-carboxylate (485 mg, 1.46 mmol), ethyl bromoacetate (366 mg, 2.19 mmol), triethylamine (295 mg, 2.92 mmol) and THF (7 mL). The tube was sealed and heated to 80° C. for 1 h. The reaction mixture was cooled to room temperature and poured into a 75 mL separatory funnel containing 30 mL of saturated aqueous KH₂PO₄ solution. The aqueous layer was extracted with diethyl ether (3×20 mL) and the combined organic layers were washed with brine, dried over MgSO₄, filtered and the solvent was evaporated under reduced pressure. The title compound was obtained as a 1:1 mixture of regioisomers and used without further purification in Step 6.

### Step 6: Ethyl {5-[5-(piperazin-1-yl)pyrazin-2-yl]-2H-tetrazol-2-yl}acetate Hydrochloride

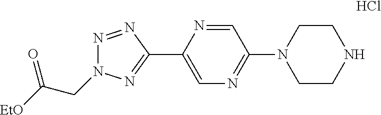

Into a 25 mL flask equipped with a magnetic stir bar was added tert-butyl-4-{5-[2-(2-ethoxy-2-oxoethyl)-2H-tetrazol-5-yl]pyrazin-2-yl}piperazine-1-carboxylate (as a 1:1 mixture of alkylated tetrazole regioisomers, 286 mg, 0.68 mmol), 4 M HCl in dioxane (2.7 mL, 10.8 mmol) and dioxane (3.4 mL). After 1 h, the solvent was evaporated under reduced pressure. The reaction mixture (a 1:1 mixture of alkylated tetrazole regioisomers) was used directly in the next step.

### Step 7: Ethyl [5-(5-{-4-[(2-bromo-5-fluorophenyl)carbonyl]piperazin-1-yl}pyrazin-2-yl)-2H-tetrazol-2-yl]acetate

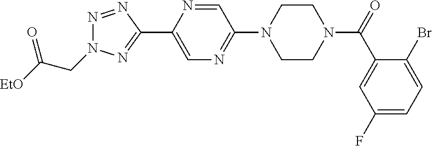

Into a 10 mL flask equipped with a magnetic stir bar was added ethyl {5-[5-(piperazin-1-yl)pyrazin-2-yl]-2H-tetrazol-2-yl}acetate hydrochloride (as a 1:1 mixture of alkylated tetrazole regioisomers, 50 mg, 0.14 mmol), 2-bromo-2-fluorobenzoyl chloride (84 mg, 0.35 mmol), triethylamine (71.3 mg, 0.71 mmol) and CH₂Cl₂ (1.4 mL). The reaction mixture was stirred at room temperature for 18 h. The reaction mixture was poured into a 75 mL separatory funnel containing 30 mL of saturated aqueous KH₂PO₄ and the aqueous layer was extracted with ethyl acetate (3×20 mL). The combined organic layers were washed with brine, dried over MgSO₄, filtered and the solvent was evaporated under reduced pressure. Purification by column chromatography through silica gel, eluting with 50:50 hexanes:EtOAc to 20:80 hexanes:EtOAc as a gradient, afforded the title compound in greater than 10:1 regioisomeric purity. MS (ESI, Q⁺) m/z 519, 521 (M+1, ⁷⁹Br, ⁸¹Br).

### Step 8: [5-(5-{4-[(2-Bromo-5-fluorophenyl)carbonyl]piperazin-1-yl}pyrazin-2-yl)-2H-tetrazol-2-yl]acetic Acid

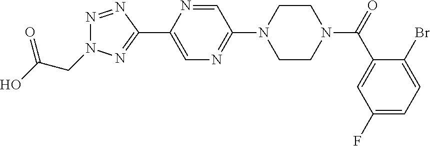

Into a 10 mL flask equipped with a magnetic stir bar was added ethyl [5-(5-{4-[(2-bromo-5-fluorophenyl)carbonyl]piperazin-1-yl}pyrazin-2-yl)-2H-tetrazol-2-yl]acetate (32 mg, 0.062 mmol), 1 N aqueous LiOH solution (0.31 mL, 0.31 mmol) and THF (0.6 mL). The solution was stirred at room temperature for 45 min, then poured into a 75 mL separatory funnel containing 30 mL of 1 M aqueous HCl solution. The aqueous layer was extracted with ethyl acetate (3×20 mL) and the combined organic layers were washed with brine, dried over MgSO₄, filtered and the solvent was evaporated under reduced pressure to afford the title compound as an off-white powder.

¹H NMR (DMSO-d₆, 400 MHz): δ 8.78 (1H, s), 8.47 (1H, s), 7.75-7.73 (1H, m), 7.41-7.39 (1H, m), 7.23-7.21 (1H, m), 5.72 (2H, s), 3.80-3.70 (6H, m), 3.31 (2H, m).

MS (ESI, Q⁺) m/z 491, 493 (M+1, ⁷⁹Br, ⁸¹Br).

### Example 2

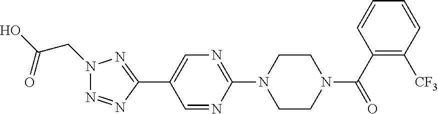

### {5-[2-(4-{[2-(Trifluoromethyl)phenyl]carbonyl}piperazin-1-yl)pyrimidin-5-yl]-2H-tetrazol-2-yl}acetic Acid

### Step 1: Ethyl {5-[2-(4-{[2-(trifluoromethyl)phenyl]carbonyl}piperazin-1-yl)pyrimidin-5-yl]-2H-tetrazol-2-yl}acetate

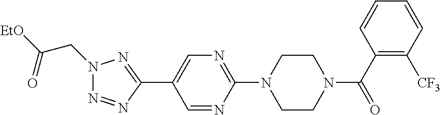

Ethyl {5-[2-(piperazin-1-yl)pyrimidin-5-yl]-2H-tetrazol-2-yl}acetate hydrochloride (Intermediate 6, 100 mg, 0.28 mmol), 2-(trifluoromethyl)benzoic acid (64 mg, 0.34 mmol), HATU (171 mg, 0.45 mmol) and DMF (4 mL) were combined in a 25 mL round-bottom flask equipped with a magnetic stir bar. The solution was treated with triethylamine (0.1 mL, 0.71 mmol) and stirred at room temperature for 4 h. The reaction mixture was diluted with water (10 mL) and CH₂Cl₂ (5 mL) and passed through a phase separatory cartridge. The aqueous layer was further extracted with CH₂Cl₂ (2×3 mL) and the combined organic layers were concentrated under reduced pressure. Purification by column chromatography through silica gel, eluting with 10% EtOAc in hexanes to 70% EtOAc in hexanes as a gradient, provided the desired product. MS (ESI, Q⁺) m/z 491 (M+1).

### Step 2: {5-[2-(4-{[2-(Trifluoromethyl)phenyl]carbonyl}piperazin-1-yl)pyrimidin-5-yl]-2H-tetrazol-2-yl}acetic Acid

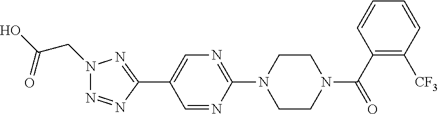

Into a 25 mL round-bottom flask equipped with a magnetic stir bar was added ethyl {5-[2-(4-{[2-(trifluoromethyl)phenyl]carbonyl}piperazin-1-yl)pyrimidin-5-yl]-2H-tetrazol-2-yl}acetate (89 mg, 0.18 mmol), THF (1.7 mL), MeOH (0.9 mL) and 1 N aqueous LiOH solution (0.9 mL, 0.9 mmol). The solution was stirred at room temperature for 2 h and poured into a 125 mL separatory funnel containing a pH 5 buffer solution (KH₂PO₄, 50 mL). The aqueous layer was extracted with EtOAc (3×25 mL) and the combined organic layers were washed with brine, dried over MgSO₄, filtered and concentrated to a white solid.

¹HNMR (d₆-Acetone, 400 MHz): δ 8.98 (2H, s), 7.90 (1H, d, J=7.5 Hz) 7.81 (1H, t, J=7.5 Hz), 7.68 (1H, t, J=7.5 Hz), 7.54 (1H, d, J=7.5 Hz), 5.63 (2H, s), 4.08-4.02 (2H, m), 3.90-3.80 (4H, m), 3.40-3.20 (2H, m). MS (ESI, Q⁺) m/z 463 (M+1).

### Example 3

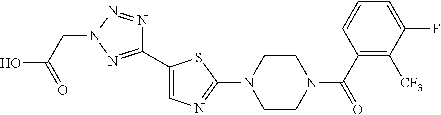

### {5-[2-(4-{[3-Fluoro-2-(trifluoromethyl)phenyl]carbonyl}piperazin-1-yl)-1,3-thiazol-5-yl]-2H-tetrazol-2-yl}acetic Acid

### Step 1: Ethyl {5-[2-(4-{[3-fluoro-2-(trifluoromethyl)phenyl]carbonyl}piperazin-1-yl)-1,3-thiazol-5-yl]-2H-tetrazol-2-yl}acetate

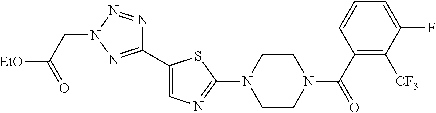

Into a 10 mL vial equipped with a magnetic stir bar was added ethyl {5-[2-(piperazin-1-yl)-1,3-thiazol-5-yl]-2H-tetrazol-2-yl}acetate hydrochloride (Intermediate 7, 75 mg, 0.208 mmol), triethylamine (0.087 mL, 0.625 mmol) and CH₂Cl₂ (2 mL). The solution was treated with 3-fluoro-2-trifluoromethylbenzoyl chloride (94 mg, 0.417 mmol) and stirred at room temperature for 16 h. The reaction mixture was placed directly onto silica gel and purified by column chromatography through silica gel, eluting with 0% EtOAc in hexanes to 50% EtOAc in hexanes as a gradient. The desired product was isolated as a white solid.

### Step 2: {5-[2-(4-{[3-Fluoro-2-(trifluoromethyl)phenyl]carbonyl}piperazin-1-yl)-1,3-thiazol-5-yl]-2H-tetrazol-2-yl}acetic Acid

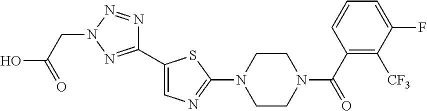

Into a 5 mL vial equipped with a magnetic stir bar was added ethyl {5-[2-(4-{[3-fluoro-2-(trifluoromethyl)phenyl]carbonyl}piperazin-1-yl)-1,3-thiazol-5-yl]-2H-tetrazol-2-yl}acetate (60 mg, 0.12 mmol) and THF (3 mL). The solution was treated with 1 N aqueous LiOH (0.58 mL, 0.58 mmol) and stirred at room temperature for 2 h. The reaction mixture was concentrated and the residue was acidified with 1 N aqueous HCl to pH 2. The resulting milky white suspension was filtered through filter paper, washing with water (1 mL) and diethyl ether (1 mL). The solid was dried under vacuum for 2 h, affording the title compound.

¹H NMR (d₆-Acetone, 400 MHz): δ 7.88-7.83 (2H, m), 7.51 (1H, t, J=10.0 Hz), 7.39 (1H, d, J=7.5 Hz), 5.56 (2H, s), 4.02-3.87 (2H, m), 3.75-3.73 (1H, m), 3.64-3.46 (3H, m).

MS (ESI, Q⁺) m/z 486 (M+1).

### Example 4

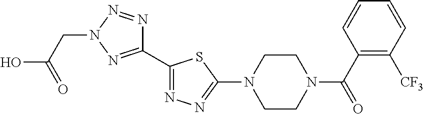

### {5-[5-(4-{[2-(Trifluoromethyl)phenyl]carbonyl}piperazin-1-yl)-1,3,4-thiadiazol-2-yl]-2H-tetrazol-2-yl}acetic Acid

### Step 1: Ethyl {5-[5-(4-{[2-(trifluoromethyl)phenyl]carbonyl}piperazin-1-yl)-1,3,4-thiadiazol-2-yl]-2H-tetrazol-2-yl}acetate

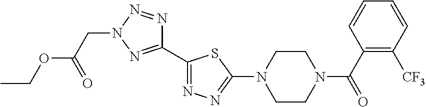

Into a 5 mL vial equipped with a magnetic stir bar was added ethyl {5-[5-(piperazin-1-yl)-1,3,4-thiadiazol-2-yl]-2H-tetrazol-2-yl}acetate hydrochloride (Intermediate 8, 100 mg, 0.277 mmol) and CH₂Cl₂ (3.0 mL). The mixture was treated with triethylamine (0.097 mL, 0.693 mmol) and then 2-trifluoromethylbenzoyl chloride (72 mg, 0.346 mmol) was added dropwise over 5 min and the mixture stirred at room temperature for 16 h. The reaction mixture was diluted with 5 mL of saturated aqueous NH₄Cl solution and poured into a phase separator cartridge, extracting with dichloromethane (2×5 mL). The organic layer was concentrated and purified by column chromatography through silica gel, eluting with 25% EtOAc in hexanes to 100% EtOAc in hexanes as a gradient. The title compound was isolated as a white foam. MS (ESI, Q⁺) m/z 497 (M+1).

### Step 2: {5-[5-(4-{[2-(Trifluoromethyl)phenyl]carbonyl}piperazin-1-yl)-1,3,4-thiadiazol-2-yl]-2H-tetrazol-2-yl}acetic Acid

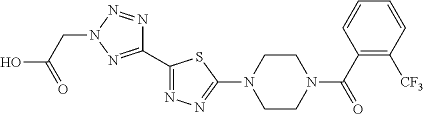

Into a 25 mL round-bottom flask equipped with a magnetic stir bar was added ethyl {5-[5-(4-[2-(trifluoromethyl)phenyl]carbonyl piperazin-1-yl)-1,3,4-thiadiazol-2-yl]-2H-tetrazol-2-yl}acetate (100 mg, 0.20 mmol), THF (2 mL) and 1.0 M aqueous LiOH (1.0 mL, 1.00 mmol). The reaction mixture was heated to reflux for 2 h, cooled to room temperature and poured into a 125 mL separatory funnel containing 1 N aqueous HCl (30 mL) and the mixture was extracted with ethyl acetate (3×30 mL). The combined organic layers were washed with brine, dried over MgSO₄, filtered and the solvent was evaporated under reduced pressure. The title compound was obtained as a solid.

¹HNMR (d₆-Acetone, 400 MHz): δ 7.85 (1H, d, J=8.0 Hz), 7.79 (1H, t, J=7.5 Hz), 7.71 (1H, t, J=7.5 Hz), 7.59 (1H, d, J=7.5 Hz), 5.80 (2H, s), 4.07-3.91 (2H, m), 3.84-3.81 (2H, m), 3.74-3.64 (2H, m), 3.54-3.43 (2H, m). MS (ESI, Q⁺) m/z 469 (M+1).

### Example 5

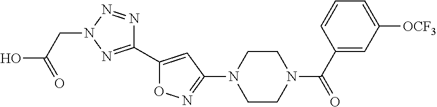

### {5-[3-(4-{[3-(Trifluoromethoxy)phenyl]carbonyl}piperazin-1-yl)isoxazol-5-yl]-2H-tetrazol-2-yl}acetic Acid

To a solution of ethyl [5-(3-piperazin-1-ylisoxazol-5-yl)-2H-tetrazol-2-yl]acetate (Intermediate 9, 20 mg, 0.065 mmol) and triethylamine (18 μL, 0.130 mmol) in THF (650 μL) was added the 3-trifluoromethoxybenzoyl chloride (22 mg, 0.098 mmol). The mixture was stirred at room temperature for 15 h then diluted with MeOH (300 μL) and 2 M aqueous NaOH solution (98 μL, 0.195 mmol). After stirring for 15 min, the mixture was acidified with acetic acid (200 μL) and the solvent was evaporated under reduced pressure. The mixture was purified directly by reverse phase (C-18) semi-prep HPLC using CH₃CN/water (+0.6% HCO₂H) as the solvent system to afford the desired product.

### Example 6

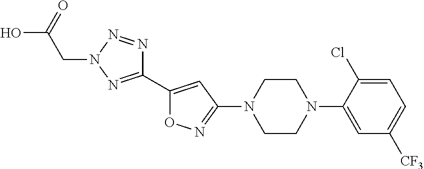

### [5-(3-{4-[2-Chloro-5-(trifluoromethyl)phenyl]piperazin-1-yl}isoxazol-5-yl)-2H-tetrazol-2-yl]acetic Acid

### Step 1: tert-Butyl 4-[2-chloro-5-(trifluoromethyl)phenyl]piperazine-1-carboxylate

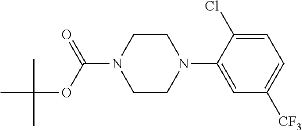

Into a 50 mL pressure vial equipped with a magnetic stir bar was added tert-butyl piperazine-1-carboxylate (2.00 g, 10.7 mmol), palladium(II) acetate (0.24 g, 1.07 mmol) and racemic-BINAP (1.34 g, 2.15 mmol). The vial was evacuated under vacuum (1 mm Hg) and backfilled with N₂ (repeated 3 times). Toluene (10 mL) and 3-bromo-4-chlorobenzotrifluoride (3.06 g, 11.8 mmol) were added to the vial and the solvent was degassed for 10 min with a steady flow of nitrogen before being heated to 120° C. for 16 h. The reaction mixture was filtered through a plug of celite on a sintered glass funnel, washing with diethyl ether (100 mL). The filtrate was concentrated and purified by column chromatography through silica gel, eluting with 0% EtOAc in hexanes to 40% EtOAc in hexanes as a gradient. The desired product was obtained as a light yellow oil.

### Step 2: 1-[2-Chloro-5-(trifluoromethyl)phenyl]piperazine Hydrochloride

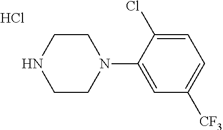

Into a 100 mL round-bottom flask equipped with a magnetic stir bar was added tert-butyl 4-[2-chloro-5-(trifluoromethyl)phenyl]piperazine-1-carboxylate (3.00 g, 8.22 mmol) and 4.0 M HCl in dioxane (20 mL, 82 mmol). The resulting mixture was stirred at room temperature for 1 h. The suspension was diluted with diethyl ether (5 mL) and filtered through filter paper on a Hirsch funnel, washing with diethyl ether (5 mL). The title compound was obtained as a light yellow solid which was dried under vacuum for 1 h.

### Step 3: 3-{4-[2-Chloro-5-(trifluoromethyl)phenyl]piperazin-1-yl}isoxazole-5-carboxamide

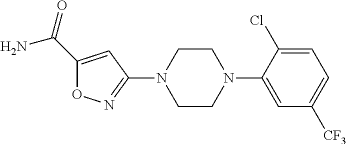

Into a 100 mL sealable pressure flask equipped with a magnetic stir bar was added 3-bromo-4,5-dihydroisoxazole-5-carboxamide (Intermediate 3, 900 mg, 4.66 mmol), 1-[2-chloro-5-(trifluoromethyl)phenyl]piperazine hydrochloride (1.4 g, 4.66 mmol) and sodium carbonate (1.7 g, 16.3 mmol). The solids were suspended in butan-1-ol (15 mL) and the vial was sealed. The resulting brownish suspension was heated at 110° C. for 16 h. The reaction mixture was cooled and decanted into a 250 mL round-bottom flask, washing the solid sodium carbonate at the bottom with ethyl acetate. The decanted mixture and ethyl acetate wash were concentrated under reduced pressure. Into a 250 mL round-bottom flask equipped with a reflux condenser and a magnetic stir bar was added the crude reaction mixture obtained above, iodine (1.7 g, 7.00 mmol), imidazole (950 mg, 14.0 mmol) and toluene (100 mL). The resulting mixture was heated at reflux temperature for 15 h. The mixture was cooled, poured into a 250 mL separatory funnel containing water (100 mL) and extracted with ethyl acetate (3×75 mL). The combined organic layers were washed with brine, dried over MgSO₄, filtered and the solvent was evaporated under reduced pressure. Purification by column chromatography through silica gel, eluting with 20% EtOAc in hexanes to 100% EtOAc in hexanes as a gradient, provided the title compound as a light brown solid.

### Step 4: 3-{4-[2-Chloro-5-(trifluoromethyl)phenyl]piperazin-1-yl}isoxazole-5-carbonitrile

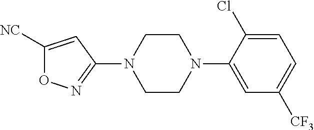

Into a 100 mL round-bottom flask equipped with a magnetic stir bar was added 3-{4-[2-chloro-5-(trifluoromethyl)phenyl]piperazin-1-yl}isoxazole-5-carboxamide (700 mg, 1.87 mmol) and THF (20 mL). The solution was cooled to 0° C. and triethylamine (1.1 mL, 7.50 mmol) was added followed by dropwise addition of TFAA (0.53 mL, 3.75 mmol). The resulting yellow solution was stirred at 0° C. for 20 min and then warmed to room temperature for 20 min and the reaction mixture was quenched with dropwise addition of saturated aqueous NaHCO₃ (50 mL). The mixture was poured into a 250 mL separatory funnel containing saturated aqueous NaHCO₃ (75 mL) and extracted with ethyl acetate (3×50 mL). The combined organic layers were washed with brine, dried over MgSO₄, filtered and the solvent was evaporated under reduced pressure. Purification by column chromatography through silica gel, eluting with 0% EtOAc in hexanes to 50% EtOAc in hexanes as a gradient, afforded the desired product as a light yellow oil.

### Step 5: 1-[2-Chloro-5-(trifluoromethyl)phenyl]-4-[5-(2H-tetrazol-5-yl)isoxazol-3-yl]piperazine

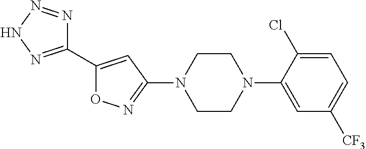

Into a 25 mL pressure flask equipped with a magnetic stir bar was added 3-{4-[2-chloro-5-(trifluoromethyl)phenyl]piperazin-1-yl}isoxazole-5-carbonitrile (620 mg, 1.70 mmol), sodium azide (560 mg, 8.70 mmol), ammonium chloride (460 mg, 8.70 mmol), dioxane (5 mL) and DMSO (0.5 mL). The resulting vial was sealed and the mixture was heated to 110° C. for 16 h. The cooled mixture was poured into a 125 mL flask and treated with 1 N aqueous HCl solution then stirred for 1 h, becoming a suspension. The beige suspension was filtered through filter paper on a Hirsch funnel, washing with water (2×5 mL). The resulting beige solid was co-evaporated with methanol to remove water and dried under vacuum for 2 h.

MS (ESI, Q⁺) m/z 400 (M+1).

### Step 6: tert-Butyl [5-(3-{4-[2-chloro-5-(trifluoromethyl)phenyl]piperazin-1-yl}isoxazol-5-yl)-2H-tetrazol-2-yl]acetate

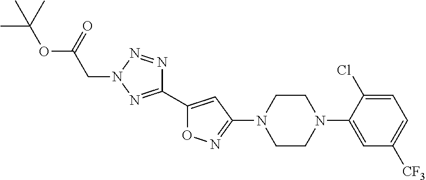

Into a 10 mL sealable pressure flask equipped with a magnetic stir bar was added 1-[2-chloro-5-(trifluoromethyl)phenyl]-4-[5-(2H-tetrazol-5-yl)isoxazol-3-yl]piperazine (400 mg, 1.00 mmol) and THF (5 mL). The solution was treated with triethylamine (0.42 mL, 3.00 mmol) and tert-butyl bromoacetate (0.30 mL, 2.00 mmol) and the vial was sealed and heated to 80° C. for 1 h. The cooled suspension was poured into a 250 mL separatory funnel containing water (75 mL) and the mixture was extracted with ethyl acetate (3×50 mL). The combined organic layers were washed with brine, dried over MgSO₄, filtered and the solvent was evaporated under reduced pressure. Purification by column chromatography through silica gel, eluting with 25% diethyl ether in hexanes to 80% diethyl ether in hexanes as a gradient, afforded the desired product as a single regioisomer.

### Step 7: [5-(3-{4-[2-Chloro-5-(trifluoromethyl)phenyl]piperazin-1-yl}isoxazol-5-yl)-2H-tetrazol-2-yl]acetic Acid

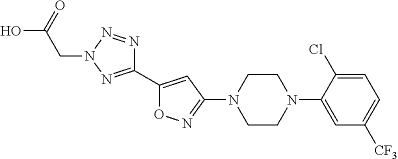

Into a 50 mL round-bottom flask equipped with a magnetic stir bar was added tert-butyl [5-(3-{4-[2-chloro-5-(trifluoromethyl)phenyl]piperazin-1-yl}isoxazol-5-yl)-2H-tetrazol-2-yl]acetate (320 mg, 0.62 mmol) and 88% aqueous formic acid (3.0 mL, 78 mmol). The resulting suspension was heated to 100° C. for 1 h, becoming a light yellow solution. The reaction was cooled to room temperature and diluted with water (20 mL). The resulting suspension was filtered through filter paper on a Hirsch funnel, washing with water (2 mL), and the solid was co-evaporated with methanol and dried under vacuum to provide the desired compound as a solid.

¹H NMR (d₆-DMSO, 400 MHz): δ 7.71 (1H, bs), 7.47 (2H, bs), 7.31 (1H, bs), 5.86 (2H, s), 3.18 (4H, bs), 2.51 (4H, bs). MS (ESI, Q⁺) m/z 458 (M+1).

### Example 7

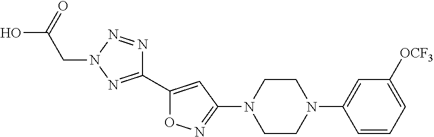

### [5-(3-{4-[3-(Trifluoromethoxy)phenyl]piperazin-1-yl}isoxazol-5-yl)-2H-tetrazol-2-yl]acetic Acid

### Step 1: 1-[3-(Trifluoromethoxy)phenyl]piperazine

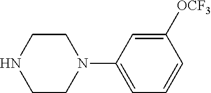

A mixture of 3-(trifluoromethoxy)aniline (2.13 g, 12.0 mmol), bis(2-chloroethyl)amine hydrochloride (2.14 g, 12.00 mmol) and 2-(2-ethoxyethoxy)ethanol (3.0 mL) was heated at 160° C. for 6 h. After being cooled to room temperature, the mixture was poured into a 250 mL separatory funnel containing aqueous 1 N NaOH solution (100 mL) and extracted with MTBE (2×50 mL). The combined organic layers were washed with brine, dried over MgSO₄, filtered and concentrated under reduced pressure. Purification by column chromatography through silica gel, eluting with 100% CH₂Cl₂ to 80:20:3 CH₂Cl₂:EtOH:NH₄OH, to afford the title compound as a light yellow oil. MS (ESI, Q⁺) m/z 247 (M+1).

### Step 2: 3-{4-[3-(Trifluoromethoxy)phenyl]piperazin-1-yl}-4,5-dihydroisoxazole-5-carboxamide

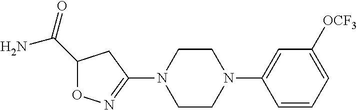

Into a 50 mL round-bottom flask was added ethanol (5 mL), 3-bromo-4,5-dihydroisoxazole-5-carboxamide (Intermediate 3, 580 mg, 3.01 mmol), 1-[3-(trifluoromethoxy)phenyl]piperazine (849 mg, 3.45 mmol) followed by DIPEA (1.58 mL, 9.02 mmol). The mixture was heated at reflux for 16 h. The mixture was poured into a 250 mL separatory funnel containing aqueous 1 N HCl solution, and the aqueous phase was extracted with EtOAc (2×50 mL). The combined organic layers were washed with aqueous 1 N HCl solution (50 mL), brine, dried over Na₂SO₄, filtered and concentrated under reduced pressure. The residue was purified by column chromatography through silica gel eluting with 0% EtOAc in hexanes to 100% EtOAc as a gradient, to afford the title compound as a white solid.

### Step 3: 3-{4-[3-(Trifluoromethoxy)phenyl]piperazin-1-yl}isoxazole-5-carboxamide

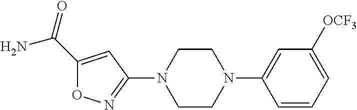

To a stirred suspension of 3-{4-[3-(trifluoromethoxy)phenyl]piperazin-1-yl}-4,5-dihydroisoxazole-5-carboxamide (704 mg, 1.96 mmol) and NaOAc (484 mg, 5.89 mmol) in chlorobenzene (6 mL) was added iodine (573 mg, 2.26 mmol). The mixture was refluxed for 6 h. An additional portion of iodine (249 mg, 0.98 mmol) was added and heating was pursued for an additional 3 h. The mixture was cooled to room temperature and diluted with a saturated aqueous Na₂S₂O₃ solution (50 mL), and EtOAc (50 mL). The mixture was stirred for about 5 min and filtered through a pad of celite on a sintered glass funnel. The filtrate was poured into a 250 mL separatory funnel and the organic layer was separated, washed with brine, dried over Na₂SO₄ and concentrated under reduced pressure. Purification by column chromatography through silica gel, eluting with 0% EtOAc in hexanes to 95% EtOAc in hexanes as a gradient, afforded the title compound as a brown solid. MS (ESI, Q⁺) m/z 357 (M+1).

### Step 4: 3-{4-[3-(Trifluoromethoxy)phenyl]piperazin-1-yl}isoxazole-5-carbonitrile

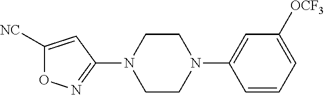

A suspension of 3-{4-[3-(trifluoromethoxy)phenyl]piperazin-1-yl}isoxazole-5-carboxamide (200 mg, 0.56 mmol) and DIPEA (0.98 mL, 5.61 mmol) in CH₂Cl₂ (4.0 mL) was cooled to −78° C. TFAA (0.12 mL, 0.84 mmol) was added dropwise to the solution and the reaction mixture was warmed slowly to 0° C. over 30 min. The reaction mixture was poured into a 250 mL separatory funnel containing saturated aqueous NH₄Cl solution, and extracted with EtOAc (3×50 mL). The combined organic layers were washed with brine, dried over Na₂SO₄, filtered and concentrated under reduced pressure. Purification by column chromatography through silica gel, eluting with 100% toluene, afforded the title compound as colorless oil.

### Step 5: 1-[5-(2H-Tetrazol-5-yl)isoxazol-3-yl]-4-[3-(trifluoromethoxy)phenyl]piperazine

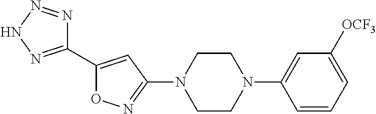

A suspension of 3-{4-[3-(trifluoromethoxy)phenyl]piperazin-1-yl}isoxazole-5-carbonitrile (157 mg, 0.464 mmol), NaN₃ (54 mg, 0.835 mmol) and NH₄Cl (74 mg, 1.39 mmol) in DMF (2 mL) was heated to 75° C. for 2 h. The reaction mixture was diluted with EtOAc, poured into a 125 mL separatory funnel containing aqueous 1 N HCl solution (50 mL), and the aqueous layer was extracted with EtOAc (2×50 mL). The combined organic layers were washed with brine, dried over Na₂SO₄, filtered and concentrated under reduced pressure to afford the title compound as a white solid. MS (ESI, Q⁺) m/z 382 (M+1).

### Step 6: [5-(3-{4-[3-(Trifluoromethoxy)phenyl]piperazin-1-yl}isoxazol-5-yl)-2H-tetrazol-2-yl]acetic Acid

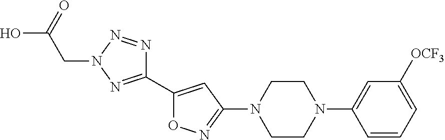

To a solution of 1-[5-(2H-tetrazol-5-yl)isoxazol-3-yl]-4-[3-(trifluoromethoxy)phenyl]piperazine (155 mg, 0.406 mmol) in dioxane (2 mL) was added DIPEA (213 μL, 1.219 mmol) and ethyl bromoacetate (91 μL, 0.817 mmol). The vial was sealed and the reaction was heated at 90° C. for 1 h. The reaction mixture was poured into a 125 mL separatory funnel containing aqueous 1 N HCl solution and extracted with EtOAc (2×50 mL). The combined organic layers were washed with brine, dried over Na₂SO₄, filtered and concentrated under reduced pressure. The residue was placed in a 25 mL round-bottom flask containing THF (4 mL) and treated with aqueous 1 N NaOH solution (2 mL). After stirring for 0.5 h at room temperature, the reaction mixture was poured into a 125 mL separatory funnel containing aqueous 1 N HCl solution (50 mL) and extracted with EtOAc (3×25 mL). The combined organic layers were washed with brine, dried over Na₂SO₄, filtered and concentrated under reduced pressure. Purification by column chromatography through silica gel, eluting with 100% CH₂Cl₂ to 70:28.5:1:0.5 CH₂Cl₂:EtOH:AcOH:H₂O as a gradient. After concentration and co-evaporation with Et₂O/heptane, the same aqueous work-up as described above was performed. The title compound was recrystallized from Et₂O/MTBE to afford a white solid. ¹H NMR (d₆-DMSO, 400 MHz): 13.92 (1H, bs), 7.39-7.33 (2H, m), 7.05 (1H, d, J=8.5 Hz), 6.96 (1H, s), 6.77 (1H, d, J=8.0 Hz), 5.85 (2H, s), 3.52-3.45 (4H, m), 3.40-3.30 (4H, m).

MS (ESI, Q⁺) m/z 440 (M+1).

The minor regioisomer (less polar acid) was isolated as a tan solid:

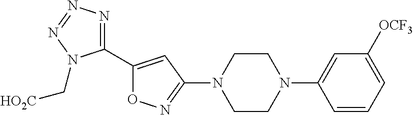

[5-(3-{4-[3-(trifluoromethoxy)phenyl]piperazin-1-yl}isoxazol-5-yl)-1H-tetrazol-1-yl]acetic acid. ¹H NMR (d₆-DMSO, 400 MHz): δ 13.89 (1H, bs), 7.49 (1H, s), 7.36 (1H, t, J=8.5 Hz), 7.05 (1H, dd, J=8.5, 2.5 Hz), 6.97 (1H, s), 6.77 (1H, d, J=8.0 Hz), 5.68 (2H, s), 3.51-3.45 (4H, m), 3.38-3.31 (4H, m). MS (ESI, Q⁺) m/z 440 (M+1).

### Example 8

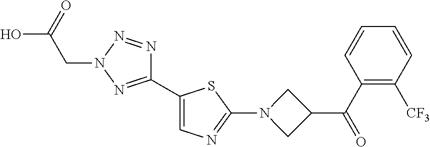

### [5-(2-{3-[2-(Trifluoromethyl)benzoyl]azetidin-1-yl}-1,3-thiazol-5-yl)-2H-tetrazol-2-yl]acetic Acid

### Step 1: tert-Butyl 3-{[methoxy(methyl)amino]carbonyl}azetidine-1-carboxylate

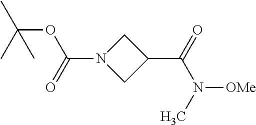

To a solution of 1-(tert-butoxycarbonyl)azetidine-3-carboxylic acid (3.78 g, 18.8 mmol), N,O-dimethylhydroxylamine hydrochloride (2.75 g, 28.2 mmol), and Et₃N (7.85 mL, 56.4 mmol) was added HATU (7.86 g, 20.7 mmol). The resulting mixture was stirred at room temperature for 19 h. A second portion of HATU (4.5 g, 11.8 mmol) was added and the reaction was stirred at room temperature for 19 h. The mixture was poured into a 250 mL separatory funnel containing water (150 mL) and extracted with EtOAc (2×50 mL). The combined organic layers were washed with water, brine, dried over MgSO₄, filtered, and concentrated under reduced pressure. The residue was purified by column chromatography through silica gel, eluting with 20% EtOAc in hexanes to 70% EtOAc in hexanes as a gradient, to afford the title compound as a colorless oil.

### Step 2: tert-Butyl 3-[2-(trifluoromethyl)benzoyl]azetidine-1-carboxylate

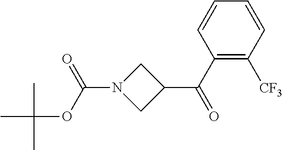

To a solution of 1-bromo-2-(trifluoromethyl)benzene (1.01 g, 4.5 mmol) and TMEDA (1.36 mL, 9.0 mmol) in THF (20 mL) at −78° C. was added slowly a solution of tert-butyl lithium (1.7 M in hexanes, 5.3 mL, 9.0 mmol). After stirring at −78° C. for 0.5 h, a solution of the product of tert-butyl 3-{[methoxy(methyl)amino]carbonyl}azetidine-1-carboxylate (1.0 g, 4.1 mmol) in THF (5 mL) was added via syringe and the reaction mixture was allowed to warm to room temperature. After 6 h, the reaction was quenched by the addition of saturated aqueous NH₄Cl solution (5 mL). The mixture was poured into a 250 mL separatory funnel containing saturated aqueous NH₄Cl solution (100 mL) and extracted with EtOAc (3×50 mL). The combined organic layers were washed with brine, dried over MgSO₄, filtered, and concentrated under reduced pressure. Purification by column chromatography through silica gel, eluting with 5% EtOAc in hexanes to 25% EtOAc in hexanes as a gradient, afforded the title compound.

### Step 3: Azetidin-3-yl[2-(trifluoromethyl)phenyl]methanone Hydrochloride

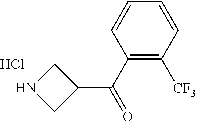

To a 25 mL round-bottom flask containing tert-butyl 3-[2-(trifluoromethyl)benzoyl]azetidine-1-carboxylate (170 mg, 0.52 mmol) was added 4 M HCl in dioxane (1.3 mL, 5.2 mmol). The mixture was stirred at room temperature for 3 h, and then concentrated under reduced pressure and co-evaporated with CH₂Cl₂ to give the title compound as a solid.

### Step 4: Ethyl [5-(2-{3-[2-(trifluoromethyl)benzoyl]azetidin-1-yl}-1,3-thiazol-5-yl)-2H-tetrazol-2-yl]acetate

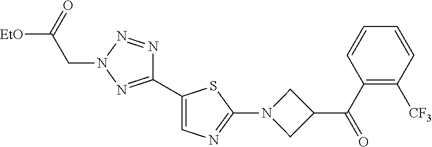

To a 5 mL microwave vial was added azetidin-3-yl[2-(trifluoromethyl)phenyl]methanone hydrochloride (60 mg, 0.23 mmol), ethyl [5-(2-bromo-1,3-thiazol-5-yl)-2H-tetrazol-2-yl]acetate (Intermediate 1, 72 mg, 0.23 mmol), NMP (2 mL), and DBU (51 μL, 0.34 mmol). The vial was sealed and heated in a microwave reactor for 15 min at 120° C. The reaction was poured into a 75 mL separatory funnel containing water (10 mL), and extracted with 3:1 EtOAc/Et₂O (25 mL). The organic layer was washed with brine, dried over MgSO₄, filtered, and concentrated under reduced pressure. The crude reaction mixture was purified by column chromatography through silica gel, eluting 5% EtOAc in hexanes to 40% EtOAc in hexanes as a gradient. The desired product was stirred for 16 h in 1:10 EtOAc/hexanes (5 mL) to afford, after filtration, a pale yellow solid.

### Step 5: [5-(2-[3-[2-(Trifluoromethyl)benzoyl]azetidin-1-yl]-1,3-thiazol-5-yl)-2H-tetrazol-2-yl]acetic Acid

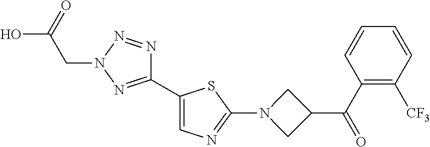

To a solution of ethyl [5-(2-{3-[2-(trifluoromethyl)benzoyl]azetidin-1-yl}-1,3-thiazol-5-yl)-2H-tetrazol-2-yl]acetate (39 mg, 0.084 mmol) in THF (1 mL) was added 1.0 N aqueous LiOH solution (167 μL, 0.167 mmol). The solution was stirred at room temperature for 2 h, and then acetic acid (30 μL) was added. The mixture was concentrated under reduced pressure, and the residue was partitioned between CH₂Cl₂ (5 mL) and water (2 mL) and separated using a phase separatory cartridge. The organic layer was concentrated under reduced pressure and the residue was stirred in 1:10 EtOAc/hexanes (3 mL) for 2 h to afford, after filtration, a white solid.

¹H NMR (Acetone-d₆, 400 MHz): δ 7.94-7.91 (1H, m), 7.90-7.79 (4H, m), 5.66 (2H, s), 4.74-4.65 (1H, m), 4.48-4.43 (2H, m), 4.38-4.33 (2H, m). MS (ESI, Q⁺) m/z 439 (M+1).

### Example 9

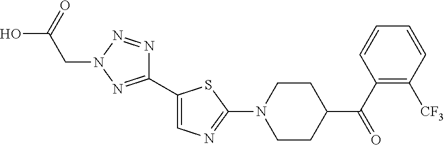

### {5-[2-(4-{[2-(Trifluoromethyl)phenyl]carbonyl}piperidin-1-yl)-1,3-thiazol-5-yl]-2H-tetrazol-2-yl}acetic Acid

### Step 1: tert-Butyl-4-{[2-(trifluoromethyl)phenyl]carbonyl}piperidine-1-carboxylate

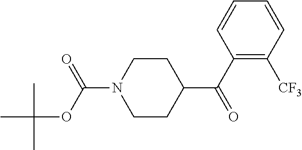

Into a 250 mL flask equipped with a magnetic stir bar was added 2-bromobenzotrifluoride (3.6 g, 16.2 mmol) and THF (30 mL). The reaction mixture was cooled to −78° C. and tert-butyllithium (1.7 M in pentanes, 19.0 mL, 32.3 mmol) was added dropwise over 10 min. After stirring at −78° C. for 0.5 h, a solution of tert-butyl 4-(methoxy(methyl)carbamoyl)piperidine-1-carboxylate (4.0 g, 14.7 mmol) in THF (5 mL) was added to the reaction mixture. At the end of the addition, the cold bath was removed and the reaction mixture was warmed to room temperature and stirred at this temperature for 1.5 h. The reaction mixture was poured into a 500 mL separatory funnel containing 10% aqueous HCl (200 mL) and extracted with ethyl acetate (3×100 mL). The combined organic layers were washed with brine, dried over MgSO₄, filtered and the solvent was evaporated under reduced pressure. Purification by column chromatography through silica gel, eluting with 20% EtOAc in hexanes to 45% EtOAc in hexanes as a gradient, afforded the title compound as a clear oil.

### Step 2: Piperidin-4-yl[2-(trifluoromethyl)phenyl]methanone Hydrochloride

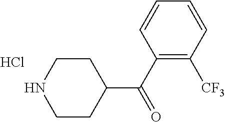

Into a 100 mL flask equipped with a magnetic stir bar was added tert-butyl-4-{[2-(trifluoromethyl)phenyl]carbonyl}piperidine-1-carboxylate (3.0 g, 8.4 mmol), 4 M HCl in dioxane (10.5 mL) and dioxane (17 mL). The reaction mixture was stirred at room temperature for 16 h. The solvent was evaporated under reduced pressure to afford the title compound as a white solid.

¹H NMR δ (DMSO-d₆, 400 MHz): δ 8.91-8.36 (2H, br s), 7.89 (1H, d, J=8.0 Hz), 7.83 (2H, d, J=4.5 Hz), 7.77 (1H, dd, J=8.0, 4.0 Hz), 3.48-3.45 (1H, m), 3.40-3.24 (2H, m), 2.93 (2H, t, J=12.5 Hz), 1.95 (2H, d, J=14.0 Hz), 1.70 (2H, t, J=12.5 Hz). MS (ESI, Q⁺) m/z 258 (M+1).

### Step 3: tert-Butyl {5-[2-(4-{[2-(trifluoromethyl)phenyl]carbonyl}piperidin-1-yl)-1,3-thiazol-5-yl]-2H-tetrazol-2-yl}acetate

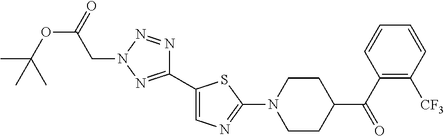

tert-Butyl {5-[2-(4-{[2-(trifluoromethyl)phenyl]carbonyl}piperidin-1-yl)-1,3-thiazol-5-yl]-2H-tetrazol-2-yl}acetate was prepared following the procedure described in Step 4 of Example 8, but using Intermediate 2 to afford the title compound as a yellow oil.

### Step 4: {5-[2-(4-{[2-(Trifluoromethyl)phenyl]carbonyl}piperidin-1-yl)-1,3-thiazol-5-yl]-2H-tetrazol-2-yl}acetic Acid

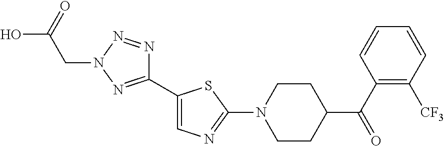

{5-[2-(4-{[2-(Trifluoromethyl)phenyl]carbonyl}piperidin-1-yl)-1,3-thiazol-5-yl]-2-H-tetrazol-2-yl}acetic acid was prepared following the procedure described in Step 5 of Example 8 to afford the title compound as an off-white powder.

¹H NMR (Acetone-d₆, 400 MHz): δ 7.89 (1H, d, J=7.5 Hz), 7.83-7.74 (4H, m), 5.55 (2H, s), 4.17 (2H, d, J=13.0 Hz), 3.50-0.344 (1H, m), 3.33-3.21 (4H, m), 1.83-1.75 (2H, m).

### Example 10

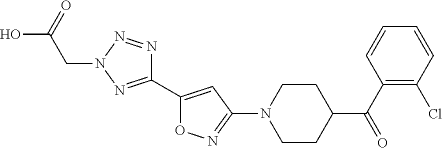

### [5-(3-{4-[(2-Chlorophenyl)carbonyl]piperidin-1-yl}isoxazol-5-yl)-2H-tetrazol-2-yl]acetic Acid

### Step 1: 1-tert-Butyl 4-pyridin-2-yl Piperidine-1,4-dicarboxylate

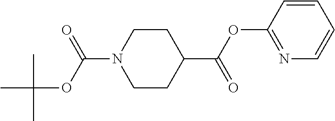

Into a 250 mL round-bottom flask equipped with a magnetic stir bar was added 1-(tert-butoxycarbonyl)piperidine-4-carboxylic acid (9.00 g, 39.3 mmol) and di-2-pyridyl carbonate (9.34 g, 43.2 mmol) in chloroform (100 mL). The solution was treated with catalytic DMAP (0.24 g, 1.96 mmol) and the reaction mixture was stirred at room temperature for 1 h. The mixture was cooled, poured into a 250 mL separatory funnel containing saturated aqueous NaHCO₃ (75 mL) and the mixture was extracted with CH₂Cl₂ (3×50 mL). The combined organic layers were washed with brine, dried over MgSO₄, filtered and the solvent was evaporated under reduced pressure. Purification by column chromatography through silica gel, eluting with 10% EtOAc in hexanes to 75% EtOAc in hexanes as a gradient, afforded the desired product as a clear oil.

### Step 2: 1-tert-Butyl 4-[(2-chlorophenyl)carbonyl]piperidine-1-carboxylate

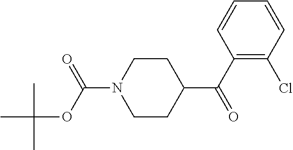

Into a 10 mL pressure vial equipped with a magnetic stir bar and under N₂ was added 1-tert-butyl 4-pyridin-2-yl piperidine-1,4-dicarboxylate (550 mg, 1.795 mmol), 2-chlorophenylboronic acid (561 mg, 3.59 mmol), palladium(II) acetate (12 mg, 0.05 mmol) and triphenylphosphine (42 mg, 0.16 mmol). The flask was evacuated under vacuum (1 mm Hg) and backfilled with N₂ (repeated 3 times). The solids were suspended in 1,4-dioxane (6 ml) and the resulting mixture was heated to 50° C. for 16 h overnight. The cooled mixture was poured into a 250 mL separatory funnel containing water (100 mL) and the mixture was extracted with ethyl acetate (3×50 mL). The combined organic layers were washed with brine, dried over MgSO₄, filtered and the solvent was evaporated under reduced pressure. Purification by column chromatography through silica gel, eluting with 0% EtOAc in hexanes to 50% EtOAc in hexanes as a gradient, afforded the title compound.

### Step 3: (2-Chlorophenyl)(piperidin-4-yl)methanone Hydrochloride

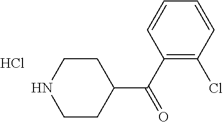

Into a 25 mL round-bottom flask equipped with a magnetic stir bar was added 1-tert-butyl 4-[(2-chlorophenyl)carbonyl]piperidine-1-carboxylate (400 mg, 1.24 mmol) and 4.0 M HCl in dioxane (3.0 mL, 12.0 mmol). The reaction mixture was stirred at room temperature for 16 h. The resulting suspension was diluted with diethyl ether (5 mL) and filtered through filter paper on a Hirsch funnel, washing with diethyl ether (5 mL). The resulting light yellow solid was dried under vacuum for 1 h. MS (ESI, Q⁺) m/z 224 (M+1).

### Step 4: tert-Butyl [5-(3-{4-[(2-chlorophenyl)carbonyl]piperidin-1-yl}-4,5-dihydroisoxazol-5-yl)-2H-tetrazol-2-yl]acetate

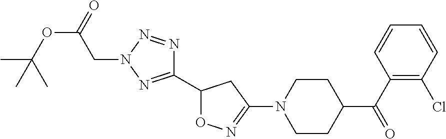

Into a 15 mL pressure flask equipped with a magnetic stir bar was added tert-butyl [5-(3-bromo-4,5-dihydroisoxazol-5-yl)-2H-tetrazol-2-yl]acetate (Intermediate 4, 299 mg, 0.90 mmol), (2-chlorophenyl)(piperidin-4-yl)methanone hydrochloride (250 mg, 0.96 mmol) and sodium bicarbonate (227 mg, 2.70 mmol). Anhydrous tert-butanol (4 mL) was added, the vial was sealed and the mixture was heated to 115° C. for 24 h. The mixture was cooled to room temperature and poured into a 125 mL separatory funnel containing water (75 mL) and the mixture extracted with ethyl acetate (3×30 mL). The combined organic layers were washed with brine, dried over MgSO₄, filtered and the solvent was evaporated under reduced pressure. Purification by column chromatography through silica gel, eluting with 10% EtOAc in hexanes to 100% EtOAc in hexanes as a gradient, afforded the title compound as an oil.

### Step 5: tent-Butyl [5-(3-{4-[(2-chlorophenyl)carbonyl]piperidin-1-yl}isoxazol-5-yl)-2H-tetrazol-2-yl]acetate

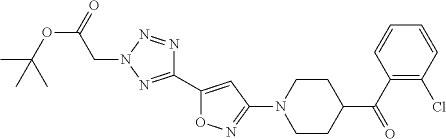

Into a 25 mL round-bottom flask equipped with a magnetic stir bar was added tert-butyl [5-(3-{4-[(2-chlorophenyl)carbonyl]piperidin-1-yl}-4,5-dihydroisoxazol-5-yl)-2H-tetrazol-2-yl]acetate (171 mg, 0.36 mmol) and THF (7 mL). The resulting solution was treated with portion wise addition of CAN (395 mg, 0.72 mmol) (added in 4 equal portions over 0.5 h). The reaction mixture was stirred an additional 0.5 h after the last addition. The mixture was cooled, poured into a 125 mL separatory funnel containing water (50 mL) and extracted with ethyl acetate (3×30 mL). The combined organic layers were washed with brine, dried over MgSO₄, filtered and the solvent was evaporated under reduced pressure. Purification by column chromatography through silica gel, eluting with 5% EtOAc in hexanes to 80% EtOAc in hexanes as a gradient, afforded the title compound. MS (ESI, Q⁺) m/z 473 (M+1).

### Step 6: [5-(3-{4-[(2-chlorophenyl)carbonyl]piperidin-1-yl}isoxazol-5-yl)-2H-tetrazol-2-yl]acetic Acid

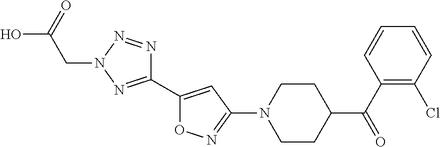

Into a 25 mL round-bottom flask equipped with a magnetic stir bar was added tert-butyl [5-(3-{4-[(2-chlorophenyl)carbonyl]piperidin-1-yl}isoxazol-5-yl)-2H-tetrazol-2-yl]acetate (61 mg, 0.13 mmol) and aqueous formic acid (2.0 mL). The resulting solution was heated to 100° C. for 1 h, and then cooled to room temperature. The mixture was treated with water (7 mL), stirred at room temperature for 10 min and filtered through filter paper on a Hirsch funnel, washing with water (2 mL). The resulting solid was co-evaporated with methanol and dried under vacuum overnight to give the desired product.

¹H NMR (d₆-DMSO, 400 MHz): δ 7.63-7.45 (4H, m), 7.21 (1H, s), 5.82 (2H, s), 4.09 (1H, bs), 3.83-3.76 (2H, m), 3.04-3.01 (2H, m), 1.88-1.85 (2H, m), 1.67-1.57 (2H, m).

MS (ESI, Q⁺) m/z 417 (M+1).

### Example 11

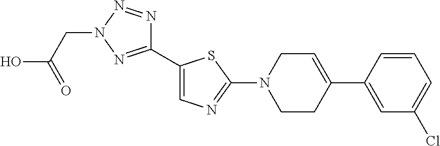

### (5-{2-[4-(3-Chlorophenyl)-3,6-dihydropyridin-1(2H)-yl]-1,3-thiazol-5-yl}-2H-tetrazol-2-yl)acetic Acid

### Step 1: tert-Butyl 4-{[(trifluoromethyl)sulfonyl]oxy}-3,6-dihydropyridine-1(2H)-carboxylate

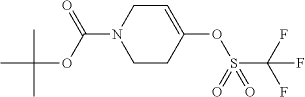

To a solution of diisopropylamine (2.36 mL, 16.6 mmol) in THF (50 mL) at −78° C. was added n-butyl lithium (1.6 M in hexanes, 10.4 mL, 16.6 mmol). After stirring 5 min at −78° C., a solution of 1-tert-butoxycarbonyl-4-piperidone (3.0 g, 15.1 mmol) in 20 mL of THF was added. The mixture was stirred for 10 min at −78° C., and a solution of N-phenyl-bis(trifluoromethanesulfonimide) (5.92 g, 16.6 mmol) in THF (30 mL) was added. After an additional 15 min at −78° C., the mixture was allowed to warm to room temperature, at which time it was quenched by the addition of saturated aqueous NaHCO₃ solution. The reaction mixture was poured into a 250 mL separatory funnel containing water (100 mL) and extracted with Et₂O (3×50 mL). The combined organic layers were washed with a 15% w/w aqueous KHSO₄ solution (50 mL), saturated aqueous NaHCO₃ solution (50 mL), brine, dried over MgSO₄, filtered, and the solvent removed under reduced pressure. Purification by column chromatography through silica gel, eluting with a gradient of 1-10% EtOAc in hexanes, afforded the desired product as a colorless oil.

### Step 2: tert-Butyl 4-(3-chlorophenyl)-3,6-dihydropyridine-1(2H)-carboxylate

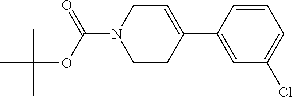

To a 5 mL pressure tube was added tert-butyl 4-{[trifluoromethyl)sulfonyl]oxy}-3,6-dihydropyridine-1(2H)-carboxylate (300 mg, 0.9 mmol), 3-chlorophenylboronic acid (142 mg, 0.91 mmol), tetrakistriphenylphosphine palladium(0) (52 mg, 0.045 mmol), and acetonitrile (2.5 mL). The mixture was degassed utilizing standard freeze/pump/thaw methods (repeated 3×), and the tube was sealed. The reaction mixture was heated at 90° C. for 1.5 h. The mixture was cooled to approximately 45° C. and filtered through a pad of celite on a sintered glass funnel. The filtrate was stirred vigorously with 25 mL of CH₂Cl₂, and passed through a phase separator cartridge to isolate the organic layer. The organics were concentrated under reduced pressure and the residue purified by column chromatography through silica gel, eluting with a gradient of 1-10% EtOAc in hexanes, to afford a colorless oil.

### Step 3: 4-(3-Chlorophenyl)-1,2,3,6-tetrahydropyridine Hydrochloride

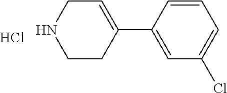

To a 25 mL round-bottom flask containing tert-butyl 4-(3-chlorophenyl)-3,6-dihydropyridine-1(2H)-carboxylate (230 mg, 0.78 mmol) was added 4 M HCl in dioxane (2 mL). The mixture was stirred at room temperature for 3 h, at which point Et₂O (10 mL) was added. After stirring an additional 1 h at room temperature, the product was isolated by filtration through filter paper on a Hirsch funnel, to afford an off-white solid.

### Step 4: tert-Butyl (5-{2-[4-(3-chlorophenyl)-3,6-dihydropyridin-1(2H)-yl]-1,3-thiazol-5-yl}-2H-tetrazol-2-yl)acetate

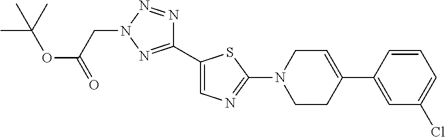

To a 2 mL microwave vial was 4-(3-chlorophenyl)-1,2,3,6-tetrahydropyridine hydrochloride (80 mg, 0.35 mmol), tert-butyl [5-(2-bromo-1,3-thiazol-5-yl)-2H-tetrazol-2-yl]acetate (Intermediate 2, 120 mg, 0.35 mmol), NMP (1.7 mL), and DIPEA (0.15 mL, 0.87 mmol). The vial was sealed and heated in a microwave reactor for 30 min at 110° C. The cooled reaction mixture was poured into a 125 mL separatory funnel containing water (10 mL) and extracted with 2:1 EtOAc/Et₂O (2:1 ratio, 30 mL). The organic layer was washed with brine, dried over MgSO₄, filtered and concentrated under reduced pressure. Purification by column chromatography through silica gel, eluting with a gradient of 5-15% EtOAc in hexanes, afforded the title compound as a pale yellow solid.

### Step 5: (5-{2-[4-(3-Chlorophenyl)-3,6-dihydropyridin-1(2H)-yl]-1,3-thiazol-5-yl}-2H-tetrazol-2-yl)acetic Acid

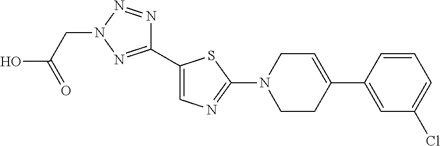

To a 25 mL round-bottom flask containing tert-butyl (5-{2-[4-(3-chlorophenyl)-3,6-dihydropyridin-1(2H)-yl]-1,3-thiazol-5-yl}-2H-tetrazol-2-yl)acetate (104 mg, 0.227 mmol) was added 88% formic acid (2 mL), and the resulting solution was stirred at 100° C. for 1.5 h. Water (5 mL) was added and the suspension was stirred for 30 min at room temperature, then filtered through filter paper under reduced pressure. After filtering, the product was dried under vacuum, and was then stirred vigourously for 1 h in 1:10 EtOAc/hexane (4 mL) and MeOH (0.5 mL) to give, after filtration, a pale green powder.

¹H NMR (DMSO-d₆, 400 MHz): δ 7.90 (1H, s), 7.55-7.53 (1H, m), 7.49-7.45 (1H, m), 7.44-7.39 (1H, m), 7.38-7.34 (1H, m), 6.39-6.36 (1H, m), 5.56 (2H, s), 4.22-4.18 (2H, m), 3.85-3.80 (2H, m), 2.71-2.66 (2H, m). MS (ESI, Q⁺) m/z 403, 405 (M+1, ³⁵Cl, ³⁷Cl).

### Example 12

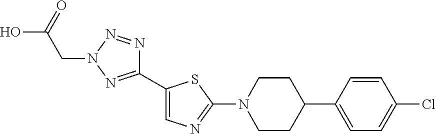

### (5-{2-[4-(4-Chlorophenyl)piperidin-1-yl]-1,3-thiazol-5-yl}-2H-tetrazol-2-yl)acetic Acid

### Step 1: Ethyl (5-{2-[4-(4-chlorophenyl)piperidin-1-yl]-1,3-thiazol-5-yl}-2H-tetrazol-2-yl)acetate

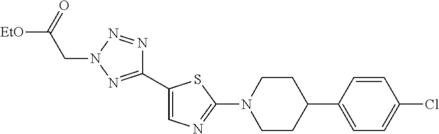

Into a 2 mL microwave vial was added ethyl [5-(2-bromo-1,3-thiazol-5-yl)-2H-tetrazol-2-yl]acetate (Intermediate 1, 85 mg, 0.27 mmol), 4-(4-chlorophenyl)piperidine (52 mg, 0.27 mmol), NMP (1.3 mL), and DBU (0.10 mL, 0.67 mmol). The vial was sealed and the reaction mixture was heated in a microwave reactor for 15 min at 120° C. The cooled reaction mixture was poured into a 125 mL separatory funnel containing water (10 mL) and extracted with EtOAc/Et₂O (3:1 ratio, 30 mL). The organic layer was further washed with brine, dried over MgSO₄, filtered and concentrated under reduced pressure. The reaction mixture was purified by column chromatography through silica gel, eluting with a gradient of 5-40% EtOAc in hexanes. The product was stirred for 16 h in 1:10 EtOAc/hexane (2 mL) to afford, after filtration, an off-white solid.

### Step 2: (5-{2-[4-(4-Chlorophenyl)piperidin-1-yl]-1,3-thiazol-5-yl}-2H-tetrazol-2-yl)acetic Acid

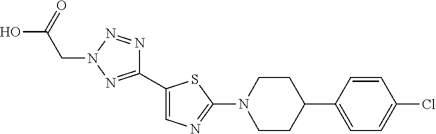

To a solution of ethyl (5-{2-[4-(4-chlorophenyl)piperidin-1-yl]-1,3-thiazol-5-yl}-2H-tetrazol-2-yl)acetate (54 mg, 0.13 mmol) in THF (1.0 mL) at room temperature was added 1.0 N aqueous LiOH solution (0.25 mL, 0.25 mmol). The reaction mixture was stirred at room temperature for 1 h. The reaction was treated with acetic acid (40 μL) and concentrated under a steady flow of N₂. The residue was poured into a 75 mL separatory funnel containing water (25 mL) and extracted with EtOAc (3×10 mL). The combined organic layers were washed with brine, dried over Na₂SO₄, filtered, and concentrated under reduced pressure. The product was stirred vigorously in 1:2 EtOAc/hexane (5 mL) for 1 h to give, after filtration, a white solid. ¹H NMR (Acetone-d₆, 400 MHz): δ 7.83 (1H, s), 7.36 (4H, s), 5.52 (2H, s), 4.26-4.19 (2H, m), 3.32-3.23 (2H, m), 2.98-2.88 (1H, m), 2.02-1.95 (2H, m), 1.91-1.79 (2H, m). MS (ESI, Q⁺) m/z 405, 407 (M+1, ³⁵Cl, ³⁷Cl).

### Example 13

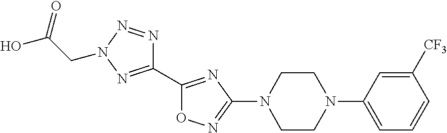

### [5-(3-{4-[3-(Trifluoromethyl)phenyl]piperazin-1-yl}-1,2,4-oxadiazol-5-yl)-2H-tetrazol-2-yl]acetic Acid

### Step 1: 4-[3-(Trifluoromethyl)phenyl]piperazine-1-carbonitrile

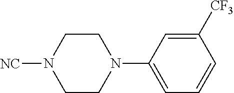

To a solution of the 1-[3-(trifluoromethyl)phenyl]piperazine hydrochloride (10.0 g, 37.5 mmol) in THF (125 mL) was added cyanogen bromide (3.97 g, 37.5 mmol), followed by triethylamine (10.5 mL, 75.0 mmol) at 0° C. The mixture was warmed to room temperature and stirred for 1 h. The solvent was evaporated under vacuum using a solvent trap and the residue diluted with water (100 mL) and aqueous 1 N HCl solution (200 mL). The mixture was poured into a separatory funnel and the aqueous layer was extracted with EtOAc (3×100 mL). The combined organic fractions were washed with water (200 mL) and dried over MgSO₄. The solvent was evaporated under reduced pressure to afford the title compound as a solid which was used in the next step without purification.

### Step 2: N′-Hydroxy-4-[3-(trifluoromethyl)phenyl]piperazine-1-carboximidamide

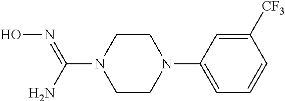

To a mixture of the 4-[3-(trifluoromethyl)phenyl]piperazine-1-carbonitrile (3.0 g, 11.8 mmol) and hydroxylamine hydrochloride (0.98 g, 14.1 mmol) in ethanol (40 mL) was added triethylamine (4.1 mL, 29.4 mmol). The mixture was stirred at room temperature for 0.5 h, and then heated to 60° C. for 1 h. The solvent was evaporated under reduced pressure and the residue was transferred to a separatory funnel using water (100 mL). The aqueous layer was extracted with EtOAc (3×50 mL), and the combined organic fractions were washed with water (100 mL), dried over MgSO₄ and evaporated under reduced pressure. The mixture was purified via trituration with Et₂O/hexanes (1:2) to afford the title compound as a solid.

### Step 3: 3-{4-[3-(Trifluoromethyl)phenyl]piperazin-1-yl}-1,2,4-oxadiazole-5-carboxamide

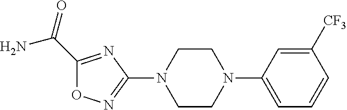

To a solution of N′-hydroxy-4-[3-(trifluoromethyl)phenyl]piperazine-1-carboximidamide (1.0 g, 3.47 mmol) and pyridine (0.84 mL, 10.41 mmol) in THF (12 mL) was added methyl oxalyl chloride (81 μL, 8.67 mmol) at 0° C. The mixture was stirred at room temperature for 1 h. The solvent was evaporated under reduced pressure, poured into a 500 mL separatory funnel and the residue diluted with 1 N aqueous HCl solution (200 mL). The aqueous layer was extracted with EtOAc (3×200 mL) and the combined organic layers were washed with brine (200 mL), dried over MgSO₄ and the solvent evaporated under reduced pressure. The crude mixture was dissolved in MeOH (12 mL), cooled to 0° C. and ammonia gas was bubbled into the solution for 5 min. The reaction mixture was stirred at room temperature for 16 h. The mixture was diluted with Et₂O (50 mL) and filtered through filter paper on a Hirsch funnel, washing with Et₂O. The filtrate was evaporated under reduced pressure and purified by column chromatography through silica gel, eluting with 60% EtOAc in hexanes. The title compound was obtained as a solid.

### Step 4: 3-{4-[3-(Trifluoromethyl)phenyl]piperazin-1-yl}-1,2,4-oxadiazole-5-carbonitrile

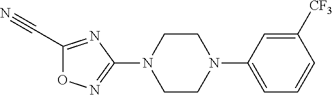

The title compound was prepared in a similar manner as that described for Intermediate 1, step 2 from 3-{4-[3-(trifluoromethyl)phenyl]piperazin-1-yl}-1,2,4-oxadiazole-5-carboxamide and TFAA.

### Step 5: 1-[5-(2H-Tetrazol-5-yl)-1,2,4-oxadiazol-3-yl]-4-[3-(trifluoromethyl)phenyl]piperazine

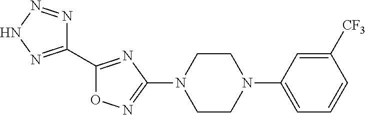

To a solution of 3-{4-[3-(trifluoromethyl)phenyl]piperazin-1-yl}-1,2,4-oxadiazole-5-carbonitrile (200 mg, 0.62 mmol) and ammonium chloride (66 mg, 1.24 mmol) in DMF (6 mL) was added sodium azide (60 mg, 0.93 mmol). The reaction mixture was heated at 100° C. for 1 h, then cooled to room temperature and diluted with water (50 mL). The aqueous layer was acidified using 1 N aqueous HCl solution and extracted with EtOAc (3×25 mL). The combined organic fractions were washed with water (50 mL) and brine (50 mL), dried with MgSO₄, filtered and evaporated under reduced pressure to afford the title compound as a solid.

### Step 6: Ethyl[5-(3-{4-[3-(trifluoromethyl)phenyl]piperazin-1-yl}-1,2,4-oxadiazol-5-yl)-2H-tetrazol-2-yl]acetate

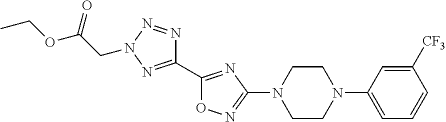

The title compound (major regioisomer) was prepared in a similar manner as that described for Intermediate 1, step 4 from 1-[5-(2H-tetrazol-5-yl)-1,2,4-oxadiazol-3-yl]-4-[3-(trifluoromethyl)phenyl]piperazine and ethylbromoacetate.

### Step 7: [5-(3-{4-[3-(Trifluoromethyl)phenyl]piperazin-1-yl}-1,2,4-oxadiazol-5-yl)-2H-tetrazol-2-yl]acetic Acid

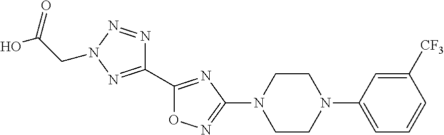

To a solution of ethyl[5-(3-{4-[3-(trifluoromethyl)phenyl]piperazin-1-yl}-1,2,4-oxadiazol-5-yl)-2H-tetrazol-2-yl]acetate (42 mg, 0.093 mmol) in THF (500 μL) was added 1 N aqueous NaOH solution (279 μL, 0.279 mmol). The reaction mixture was stirred at room temperature for 1 h and then the solvent was evaporated under reduced pressure. The residue was poured into a 75 mL separatory funnel, diluted with water (10 mL) and 1 N aqueous HCl solution (10 mL), then extracted with EtOAc (3×10 mL). The combined organic layers were dried with MgSO₄, filtered and evaporated under reduced pressure. The solid was purified by trituration in Et₂O/hexanes (1/5) to afford the title compound.

¹HNMR (DMSO-d₆, 500 MHz): δ 7.47 (1H, t, J=8.0 Hz), 7.31 (1H, d, J=8.5 Hz), 7.27 (1H, s), 7.13 (1H, d, J=7.5 Hz), 5.93 (2H, s), 3.66-3.58 (4H, m), 3.44-3.38 (4H, m).

MS (ESI, Q⁺) m/z 425 (M+1).

### Example 14

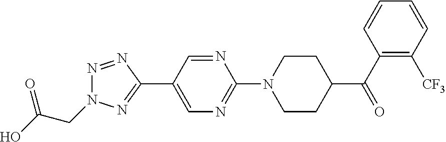

### [5-(2-{4-[2-(Trifluoromethyl)benzoyl]piperidin-1-yl}pyrimidin-5-yl)-2H-tetrazol-2-yl]acetic Acid

Ethyl [5-(2-chloropyrimidin-5-yl)-2H-tetrazol-2-yl]acetate (750 mg, 2.79 mmol) was added to a 125 mL Erlenmeyer flask and dissolved in 25 mL of dioxane, creating a 0.112 M stock solution. To a 5 mL screw top test tube was added piperidin-4-yl[2-(trifluoromethyl)phenyl]methanone (43 mg, 0.168 mmol), along with a magnetic stir bar. 1 mL of the 0.112 M stock solution was added to the test tube, followed by potassium carbonate (37 mg, 0.268 mmol). A cap was fixed tightly to the test tube, and the tube was heated on a magnetic stir plate at 70° C. for 18 h. The cooled test tube was treated with 0.56 mL of methanol and 0.56 mL of a 1 N aqueous LiOH solution. The reaction was stirred at room temperature for 16 h. The stir bar was removed and the solvent removed using a centrifugal evaporator. The residue was dissolved in 1.2 mL of DMSO and purified using mass-directed LC/MS, using a gradient of 40:60 (acetonitrile:0.5% ammonium acetate in water), to 80:20 (acetonitrile:0.5% ammonium acetate in water), and a Synergi Max-RP Axia 50X™ 21.2 mm 4 micron preparative HPLC column.

¹H NMR (d₆-DMSO, 400 MHz): δ 8.97 (2H, s), 7.87-7.70 (4H, m), 5.53 (2H, s), 4.80 (2H, d, J=13.0 Hz), 3.52 (1H, m), 3.13 (2H, d, J=12.5 Hz), 1.95 (2H, t, J=12.5 Hz), 1.51 (2H, d, J=13.0 Hz). MS (ESI, Q⁺) m/z 462 (M+1).

### Example 15

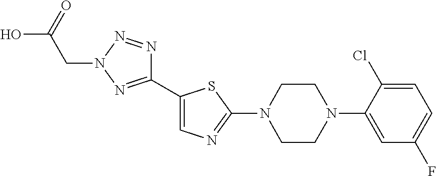

### (5-{2-[4-(2-Chloro-5-fluorophenyl)piperazin-1-yl]-1,3-thiazol-5-yl}-2H-tetrazol-2-yl)acetic Acid

### Step 1: tert-Butyl 4-(2-chloro-5-fluorophenyl)piperazine-1-carboxylate

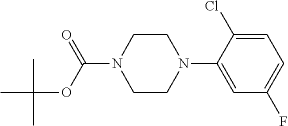

Into a 50 mL pressure vial equipped with a magnetic stir bar was added tert-butyl piperazine-1-carboxylate (2.00 g, 10.7 mmol), palladium(II) acetate (0.24 g, 1.07 mmol) and racemic-BINAP (1.33 g, 2.14 mmol). The vial was evacuated under vacuum (1 mm Hg) and backfilled with N₂ (repeated 3 times). Toluene (10 mL) and 1-bromo-2-chloro-5-fluorobenzene (2.47 g, 11.8 mmol) were added to the vial and the solvent was degassed for 10 min with a steady flow of nitrogen before being heated to 120° C. for 16 h. The reaction was filtered through a plug of celite on a sintered glass funnel, washing with diethyl ether (100 mL). The filtrate was concentrated and purified by column chromatography through silica gel, eluting with 0% EtOAc in hexanes to 40% EtOAc in hexanes as a gradient, to afford the title compound as a yellow solid.

### Step 2: 1-(2-Chloro-5-fluorophenyl)piperazine Hydrochloride

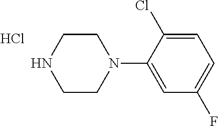

Into a 100 mL round-bottom flask equipped with a magnetic stir bar was added tert-butyl 4-(2-chloro-5-fluorophenyl)piperazine-1-carboxylate (2.68 g, 8.51 mmol) and 4.0 M HCl in dioxane (22.0 mL, 85 mmol). The resulting suspension was stirred at room temperature for 16 h. The suspension was diluted with diethyl ether (5 mL) and filtered through filter paper on a Hirsch funnel, washing with diethyl ether (2×5 mL). The resulting beige solid was dried under vacuum for 1 h to afford the title compound as the HCl salt.

MS (ESI, Q⁺) m/z 215 (M+1).

### Step 3: tert-Butyl (5-{2-[4-(2-chloro-5-fluorophenyl)piperazin-1-yl]-1,3-thiazol-5-yl}-2H-tetrazol-2-yl)acetate

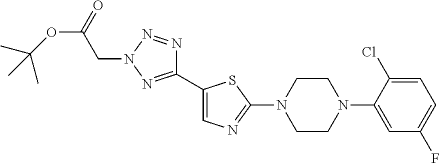

Into a 15 mL microwave vial equipped with a magnetic stirbar was added tert-butyl [5-(2-bromo-1,3-thiazol-5-yl)-2H-tetrazol-2-yl]acetate (500 mg, 1.44 mmol), 1-(2-chloro-5-fluorophenyl)piperazine hydrochloride (363 mg, 1.44 mmol), NMP (3.0 mL) and DBU (0.54 mL, 3.61 mmol). The vial was sealed and heated in a microwave reactor at 120° C. for 30 min. The cooled mixture was poured into a 125 mL separatory funnel containing water (75 mL) and the mixture was extracted with ethyl acetate (3×30 mL). The combined organic layers were washed with brine, dried over MgSO₄, filtered and the solvent was evaporated under reduced pressure. Purification by column chromatography through silica gel, eluting with 0% EtOAc in hexanes to 50% EtOAc in hexanes as a gradient, provided the title compound as an off-white solid.

### Step 4: (5-{2-[4-(2-Chloro-5-fluorophenyl)piperazin-1-yl]-1,3-thiazol-5-yl}-2H-tetrazol-2-yl)acetic Acid

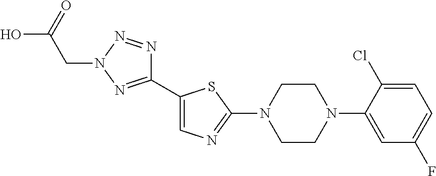

Into a 25 mL round-bottom flask equipped with a magnetic stir bar was added tert-butyl (5-{2-[4-(2-chloro-5-fluorophenyl)piperazin-1-yl]-1,3-thiazol-5-yl}-2H-tetrazol-2-yl)acetate (400 mg, 0.83 mmol) and 88% aqueous formic acid (4.0 mL, 100 mmol). The resulting solution was heated to 100° C. for 1 h. The cooled reaction mixture was diluted with water (20 mL) and filtered through filter paper on a Hirsch funnel, washing with water (1 mL). The resulting solid was co-evaporated with methanol to remove excess water and dried under vacuum to give the desired product.

¹HNMR (d₆-DMSO, 400 MHz): δ 7.92 (1H, s), 7.50 (1H, t, J=7.5 Hz), 7.09 (1H, d, J=9.0 Hz), 6.96 (1H, t, J=7.0 Hz), 5.70 (2H, s), 3.70 (4H, bs), 3.39 (4H, bs). MS (ESI, Q⁺) m/z 424 (M+1).

### Example 16

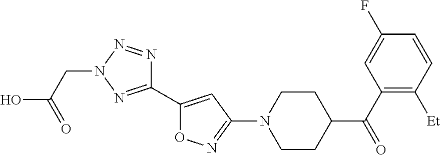

### {5-(3-[4-[(2-Ethyl-5-fluorophenyl)carbonyl]piperidin-1-yl}isoxazol-5-yl)-2H-tetrazol-2-yl]acetic acid

### Step 1: 1-(2-Bromo-4-fluorophenyl)ethanol

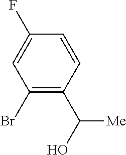

Into a flame-dried 250-mL round-bottom flask equipped with a magnetic stir bar and under N₂ was added methylmagnesium bromide (9.03 ml, 27.1 mmol, 3.0 M in diethyl ether) and diethyl ether (40 mL). The mixture was cooled to 0° C. and then a solution of 2-bromo-4-fluorobenzaldehyde (5.00 g, 24.63 mmol) in 25 mL of diethyl ether was added dropwise over 20 min. The resulting suspension was stirred at 0° C. for 2 h. The reaction mixture was quenched with dropwise addition of a saturated aqueous NH₄Cl solution (5 mL). The mixture was cooled, poured into a 250 mL reparatory funnel containing water (125 mL) and the mixture was extracted with diethyl ether (3×50 mL). The combined organic layers were washed with brine, dried over MgSO₄, filtered and the solvent was evaporated under reduced pressure. Purification by column chromatography through silica gel, eluting with 0% EtOAc in hexanes to 40% EtOAc in hexanes as a gradient afforded the title compound as a clear oil. MS (ESI, Q⁺) m/z 201, 203 (M+1).

### Step 2: 2-Bromo-4-fluoro-ethylbenzene

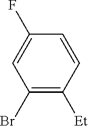

Into a 125-mL round-bottom flask equipped with a magnetic stir bar was added 1-(2-bromo-4-fluorophenyl)ethanol (4.00 g, 18.3 mmol) in hexanes (20 mL). The solution was treated with sodium iodide (16.4 g, 110 mmol) followed by dropwise addition of chlorotrimethylsilane (14.0 mL, 110 mmol). The dark reaction mixture was stirred overnight at room temperature and under an atmosphere of nitrogen. The resulting mixture was diluted with water (25 mL) and diethyl ether (50 mL). The mixture was stirred at room temperature for 15 min and then poured into a 250 mL separatory funnel containing water (100 mL) and the mixture was extracted with diethyl ether (3×75 mL). The combined organic layers were washed with sodium bisulfate (2×100 mL), brine (100 mL), dried over MgSO₄, filtered and the solvent was evaporated under reduced pressure. Purification by column chromatography through silica gel, eluting with 100% hexanes afforded the title compound as a colorless liquid.

### Step 3: tert-Butyl 4-[(2-ethyl-5-fluorophenyl)carbonyl]piperidine-1-carboxylate

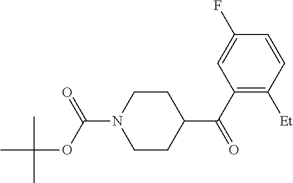

Into a flame-dried 250-mL round-bottom flask equipped with a magnetic stir bar and under N₂ was added 2-bromo-4-fluoro-ethylbenzene (3.20 g, 15.8 mmol) and tetrahydrofuran (60 mL). The solution was cooled to −78° C. and then tert-butyllithium (18.5 ml, 31.5 mmol) was added dropwise to the solution over 10 min to give a light yellow solution. This was stirred at −78° C. for 5 min and then a solution of 1-tert-butoxycarbonyl-4-(methoxy-methylcarbamoyl)piperidine (3.90 g, 14.3 mmol) in 10 mL of THF was added via cannula over 5 min. The reaction mixture was stirred at −78° C. for 5 min, the ice bath was removed and the mixture was allowed to warm to room temperature over 1 h. The reaction mixture was quenched with dropwise addition of a saturated aqueous NH₄Cl solution (5 mL) and concentrated to remove the THF. The mixture was poured into a 250 mL separatory funnel containing saturated aqueous NH₄Cl (125 mL) and the mixture was extracted with ethyl acetate (3×75 mL). The combined organic layers were washed with brine, dried over MgSO₄, filtered and the solvent was evaporated under reduced pressure. Purification by column chromatography through silica gel, eluting with 0% EtOAc in hexanes to 40% EtOAc in hexanes as a gradient afforded the desired product as a light yellow oil.

### Step 4: (2-Ethyl-5-fluorophenyl)(piperidin-4-yl)methanone Hydrochloride

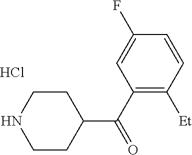

Into a 250-mL round-bottom flask equipped with a magnetic stir bar was added tert-butyl 4-[(2-ethyl-5-fluorophenyl)carbonyl]piperidine-1-carboxylate (3.10 g, 9.24 mmol), 1,4-dioxane (20 mL) and 4 M HCl in dioxane (23 mL, 92 mmol). The resulting solution was stirred at room temperature for 2 h, becoming a white suspension. The resulting suspension was diluted with diethyl ether (20 mL) and filtered through filter paper on a Hirsch funnel under vacuum and the white filter cake was washed with diethyl ether (2×3 mL). The resulting white solid was dried on the vacuum pump overnight.

### Step 5: tert-Butyl [5-(3-{4-[(2-ethyl-5-fluorophenyl)carbonyl]piperidin-1-yl}-4,5-dihydroisoxazol-5-yl)-2H-tetrazol-2-yl]acetate

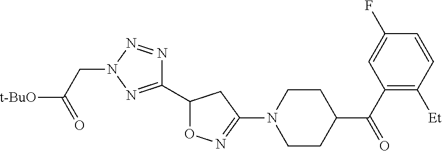

Into a 50-mL sealable flask equipped with a magnetic stir bar was added tert-butyl [5-(3-bromo-4,5-dihydroisoxazol-5-yl)-2H-tetrazol-2-yl]acetate (Intermediate 4, 750 mg, 2.26 mmol), (2-ethyl-5-fluorophenyl)(piperidin-4-yl)methanone hydrochloride (1.23 g, 4.52 mmol) and sodium bicarbonate (570 mg, 6.77 mmol). The resulting solids were suspended in anhydrous tert-butanol (20 mL) and the vial was sealed and heated to 110° C. in an oil bath for 26 h. The resulting mixture was cooled, poured into a 250 mL reparatory funnel containing water (125 mL) and the mixture was extracted with ethyl acetate (3×50 mL). The combined organic layers were washed with brine, dried over MgSO₄, filtered and the solvent was evaporated under reduced pressure. This material was used directly in the next step. MS (ESI, Q⁺) m/z 487 (M+1).

### Step 6: tert-Butyl [5-(3-{4-[(2-ethyl-5-fluorophenyl)carbonyl]piperidin-1-yl}isoxazol-5-yl)-2H-tetrazol-2-yl]acetate

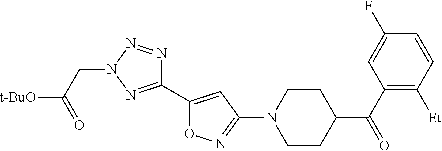

Into a 100-mL round-bottom flask equipped with a magnetic stir bar was added tert-butyl [5-(3-{4-[(2-ethyl-5-fluorophenyl)carbonyl]piperidin-1-yl}-4,5-dihydroisoxazol-5-yl)-2H-tetrazol-2-yl]acetate (1.10 g, 2.26 mmol) and sodium bicarbonate (0.57 g, 6.78 mmol) in tetrahydrofuran (20 mL). The suspension was treated with cerium ammonium nitrate (2.45 g, 4.52 mmol) added in 4 equal portions over 20 min. After one hour, the mixture was poured into a 250 mL separatory funnel containing water (75 mL) and the mixture was extracted with ethyl acetate (3×50 mL). The combined organic layers were washed with brine, dried over MgSO₄, filtered and the solvent was evaporated under reduced pressure. Purification by column chromatography through silica gel, eluting with 20% ethyl acetate in hexanes to 50% ethyl acetate in hexanes as a gradient afforded the desired product which was further purified by reverse phase chromatography using a preparative C18 column and water:acetonitrile as the mobile phase. The desired product was isolated as a light yellow oil.

MS (ESI, Q⁺) m/z 485 (M+1).

### Step 7: [5-(3-{4-[(2-Ethyl-5-fluorophenyl)carbonyl]piperidin-1-yl}isoxazol-5-yl)-2H-tetrazol-2-yl]acetic Acid

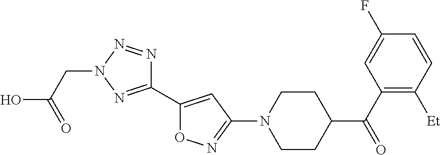

Into a 25-mL round-bottom flask equipped with a magnetic stir bar was added tert-butyl [5-(3-{4-[(2-ethyl-5-fluorophenyl)carbonyl]piperidin-1-yl}isoxazol-5-yl)-2H-tetrazol-2-yl]acetate (120 mg, 0.248 mmol) and formic acid (3.0 mL, 78 mmol). The resulting solution was heated to 100° C. for 30 min. The cooled solution was diluted with water (25 mL) and poured into a 125 mL separatory funnel containing water (25 mL) and the mixture was extracted with ethyl acetate (3×25 mL). The combined organic layers were washed with brine, dried over MgSO₄, filtered and the solvent was evaporated under reduced pressure. The desired product was isolated as an off-white solid.

¹H NMR (d₆-DMSO, 400 MHz): δ 7.59 (1H, dd, J=7.0, 2.5 Hz), 7.41-7.38 (1H, m), 7.34-7.29 (1H, m), 7.22 (1H, s), 5.84 (2H, s), 3.86-3.82 (2H, m), 3.47-3.40 (1H, m), 3.06-2.99 (2H, m), 2.62 (2H, q, J=7.5 Hz), 1.84-1.81 (2H, m), 1.63-1.53 (2H, m), 1.12 (3H, t, J=7.5 Hz). MS (ESI, Q⁺) m/z 429 (M+1).

The following additional Examples shown in the Table below were prepared following the procedures outlined in Methods A-AD and detailed in Examples 1-16.

| Prepared | | |
|---|---|---|
| following | | MS Data |
| Example | EXAMPLE | (ESI, Q⁺) |
| 2 | 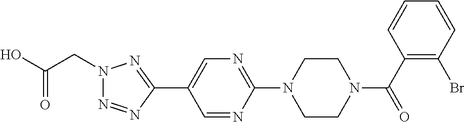 | 473, 475 (M + 1) |
| 2 | 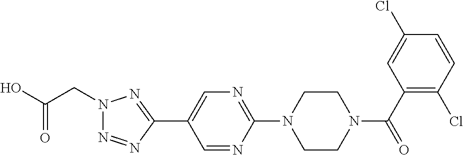 | 463 (M + 1) |
| 2 | 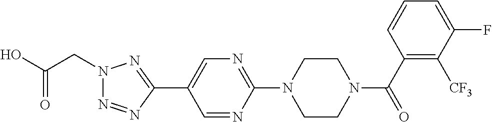 | 481 (M + 1) |
| 3 | 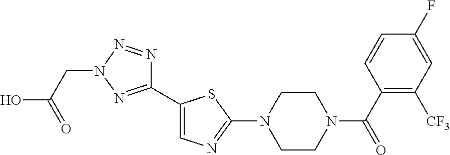 | 486 (M + 1) |
| 3 | 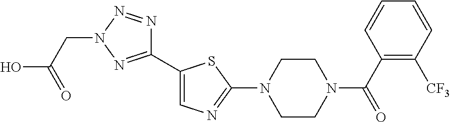 | 468 (M + 1) |
| 3 | 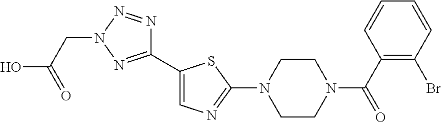 | 478, 480 (M + 1) |
| 3 | 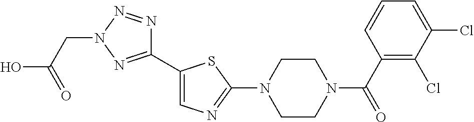 | 468 (M + 1) |
| 3 | 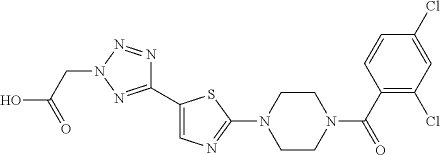 | 468 (M + 1) |
| 3 | 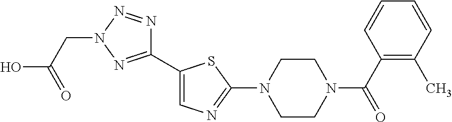 | 414 (M + 1) |
| 3 | 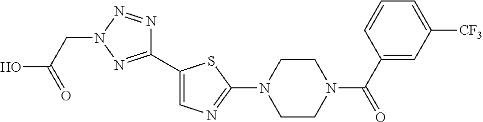 | 468 (M + 1) |
| 3 | 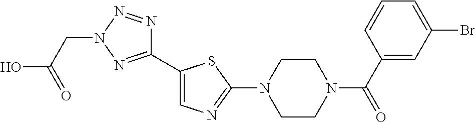 | 478, 480 (M + 1) |
| 4 | 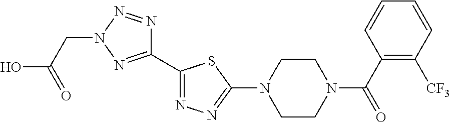 | 469 (M + 1) |
| 5 | 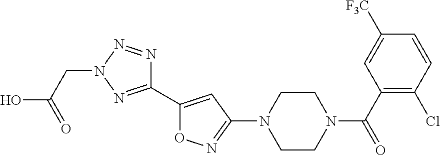 | 486 (M + 1) |
| 1 | 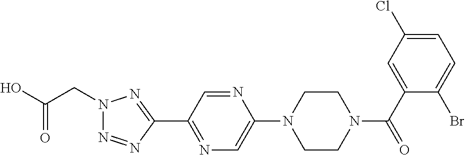 | 507 (M + 1) |
| 1 | 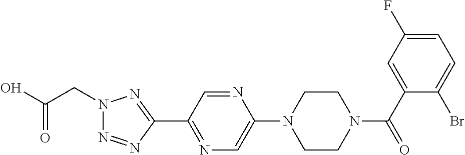 | 491, 493 (M + 1) |
| 1 | 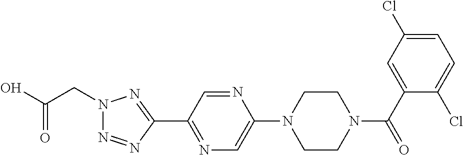 | 463 (M + 1) |
| 5 | 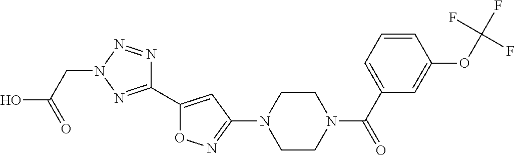 | 468 (M + 1) |
| 5 | 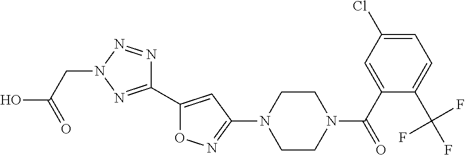 | 486 (M + 1) |
| 5 | 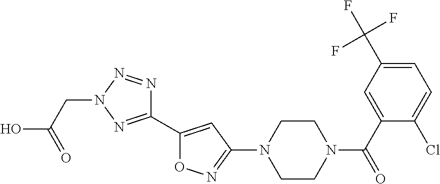 | 486 (M + 1) |
| 5 | 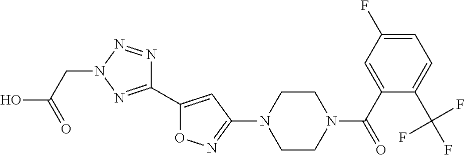 | 470 (M + 1) |
| 5 | 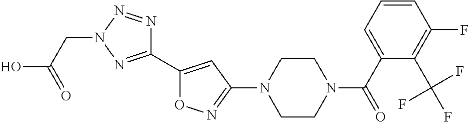 | 470 (M + 1) |
| 5 | 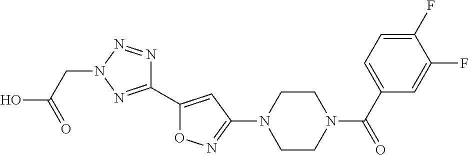 | 420 (M + 1) |
| 5 | 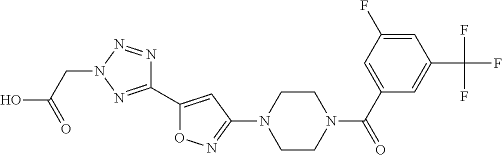 | 470 (M + 1) |
| 5 | 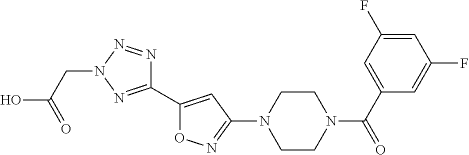 | 420 (M + 1) |
| 5 | 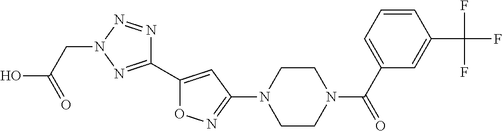 | 452 (M + 1) |
| 5 | 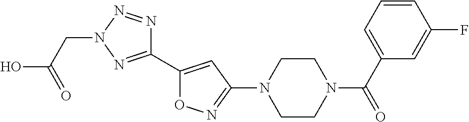 | 402 (M + 1) |
| 5 | 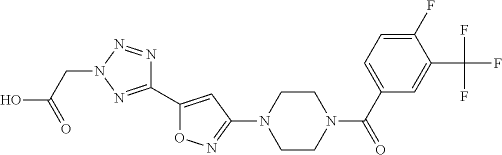 | 470 (M + 1) |
| 5 | 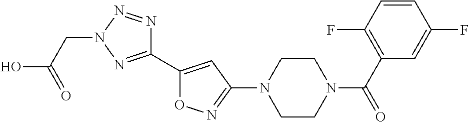 | 420 (M + 1) |
| 5 | 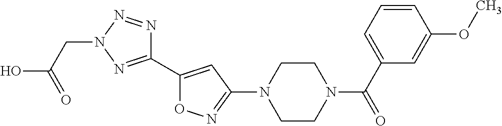 | 414 (M + 1) |
| 5 | 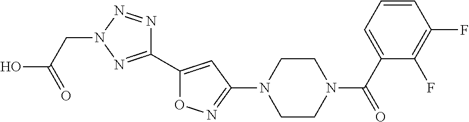 | 420 (M + 1) |
| 5 | 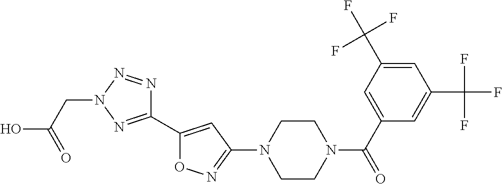 | 520 (M + 1) |
| 4 | 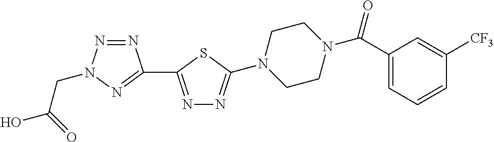 | 469 (M + 1) |
| 4 | 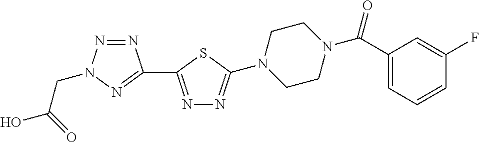 | 419 (M + 1) |
| 4 | 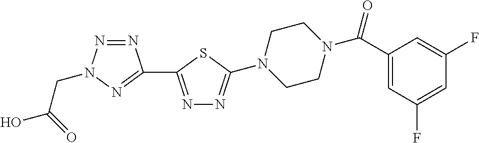 | 437 (M + 1) |
| 4 | 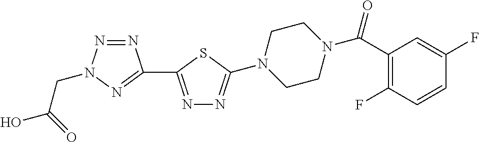 | 437 (M + 1) |
| 6 | 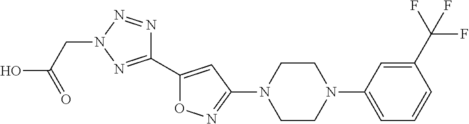 | 424 (M + 1) |
| 6 | 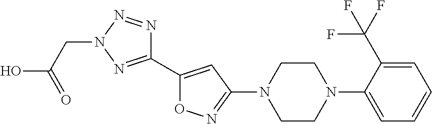 | 424 (M + 1) |
| 6 | 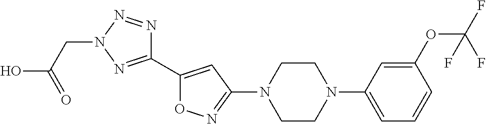 | 440 (M + 1) |
| 6 | 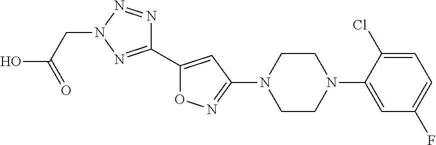 | 408 (M + 1) |
| 6 | 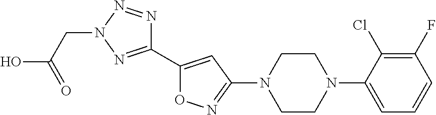 | 408 (M + 1) |
| 6 | 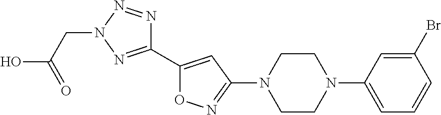 | 434 (M + 1) |
| 6 | 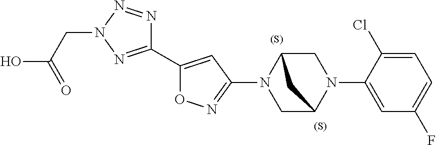 | 420 (M + 1) |
| 6 | 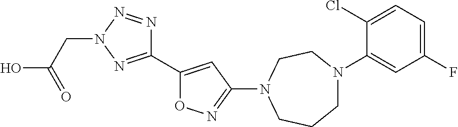 | 422 (M + 1) |
| 15 | 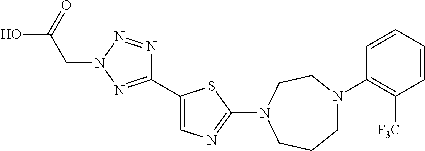 | 454 (M + 1) |
| 15 | 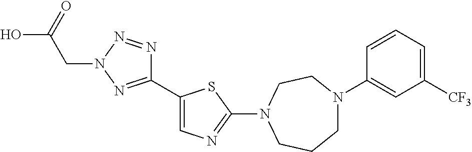 | 454 (M + 1) |
| 15 | 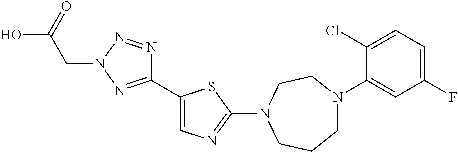 | 438 (M + 1) |
| 14 | 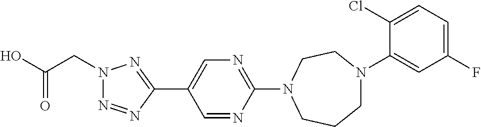 | 433 (M + 1) |
| 9 | 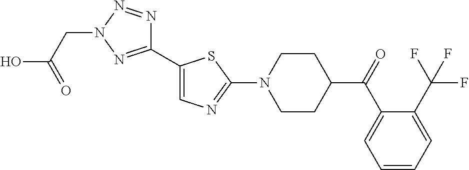 | 467 (M + 1) |
| 10 | 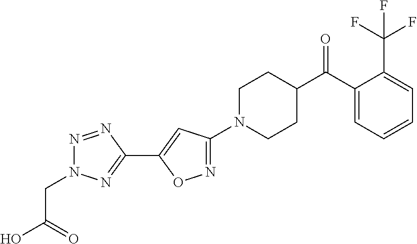 | 451 (M + 1) |
| 9 | 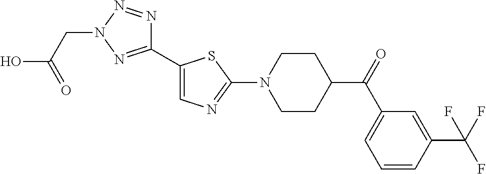 | 467 (M + 1) |
| 14 | 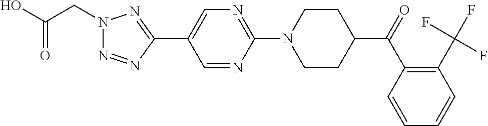 | 462 (M + 1) |
| 11 | 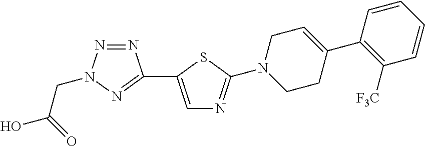 | 437 (M + 1) |
| 11 | 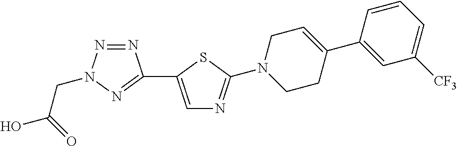 | 437 (M + 1) |
| 11 | 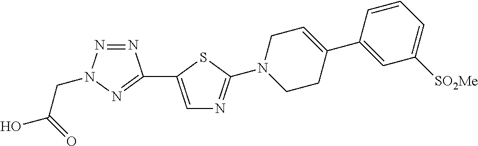 | 447 (M + 1) |
| 11 | 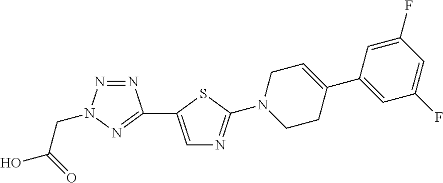 | 405 (M + 1) |
| 12 | 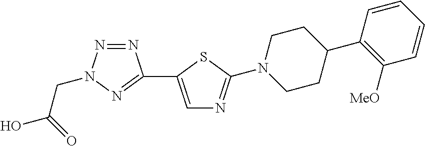 | 401 (M + 1) |
| 12 | 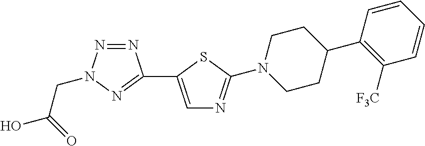 | 439 (M + 1) |
| 12 | 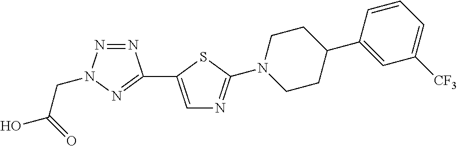 | 439 (M + 1) |
| 14 | 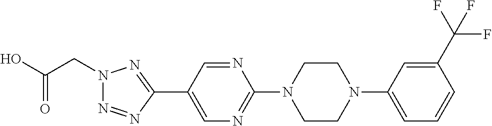 | 435 (M + 1) |
| 14 | 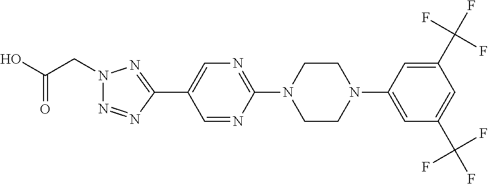 | 503 (M + 1) |
| 14 | 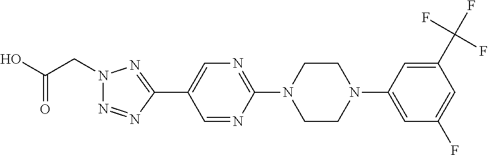 | 453 (M + 1) |
| 14 | 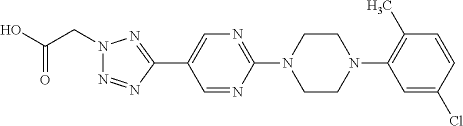 | 415 (M + 1) |
| 14 | 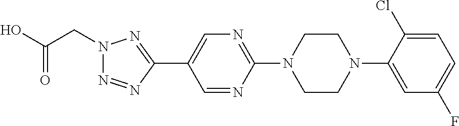 | 419 (M + 1) |
| 15 | 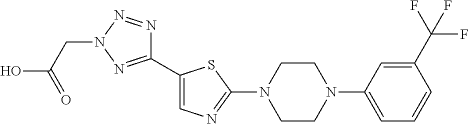 | 440 (M + 1) |
| 15 | 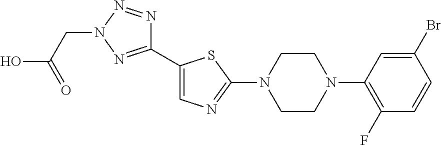 | 468, 470 (M + 1) |
| 15 | 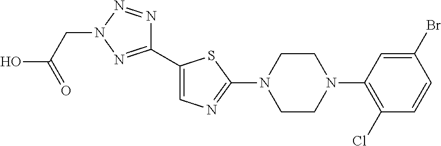 | 484, 486 (M + 1) |
| 15 | 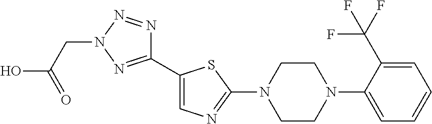 | 440 (M + 1) |
| 15 | 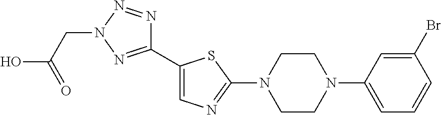 | 450, 452 (M + 1) |
| 15 | 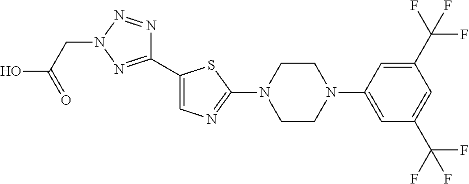 | 508 (M + 1) |
| 15 | 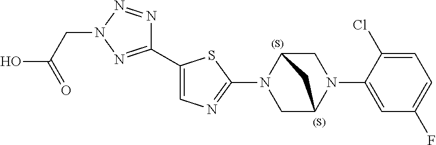 | 436 (M + 1) |
| 15 | 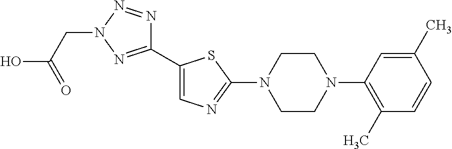 | 400 (M + 1) |
| 15 | 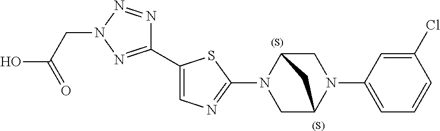 | 418 (M + 1) |
| 15 | 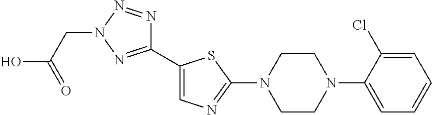 | 406 (M + 1) |
| 15 | 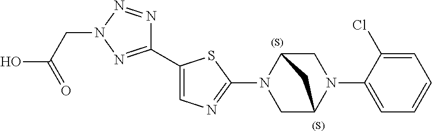 | 418 (M + 1) |
| 15 | 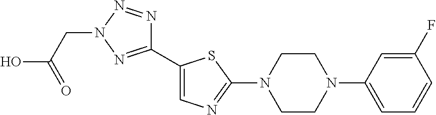 | 390 (M + 1) |
| 15 | 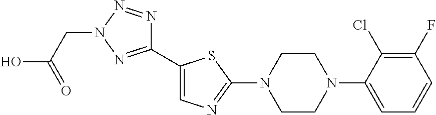 | 424 (M + 1) |
| 15 | 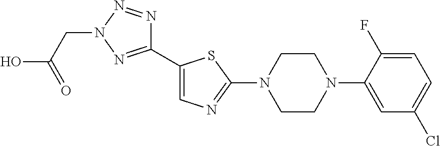 | 424 (M + 1) |
| 15 | 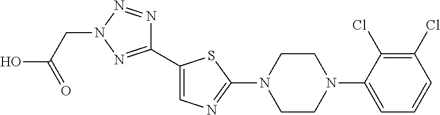 | 440 (M + 1) |
| 15 | 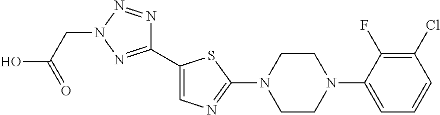 | 424 (M + 1) |
| 15 | 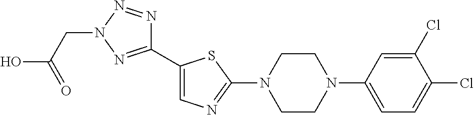 | 440 (M + 1) |
| 15 | 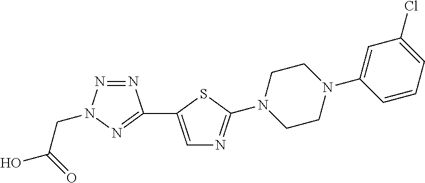 | 406 (M + 1) |
| 15 | 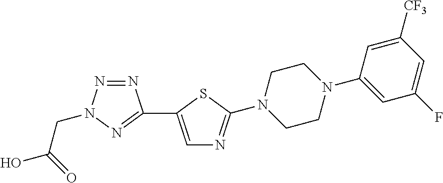 | 458 (M + 1). |
| 15 | 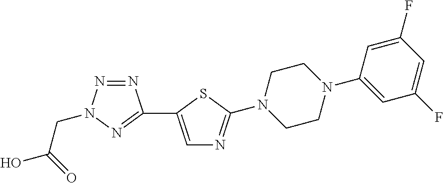 | 408 (M + 1) |
| 15 | 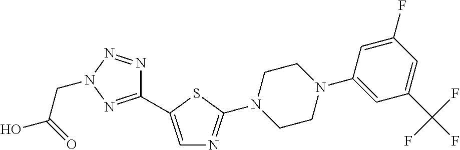 | 457 (M + 1) |
| 15 | 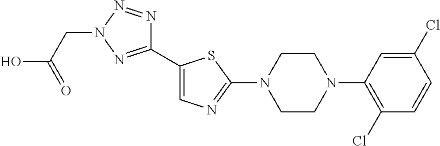 | 440 (M + 1) |
| 15 | 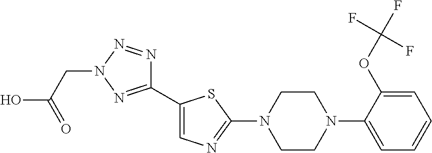 | 456 (M + 1) |
| 15 | 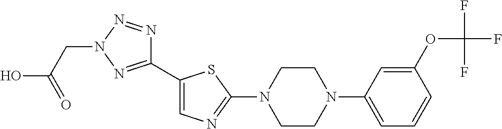 | 456 (M + 1) |
| 15 | 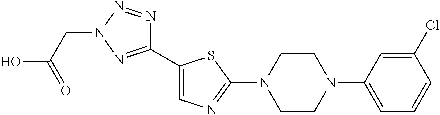 | 406 (M + 1) |
| 15 | 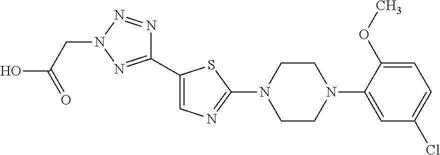 | 436 (M + 1) |
| 15 | 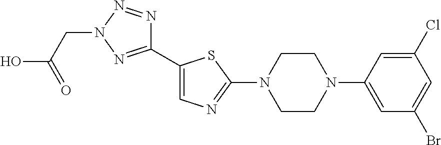 | 484, 486 (M + 1) |
| 10 | 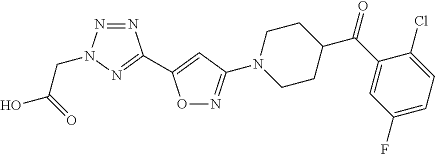 | 435 (M + 1) |
| 10 | 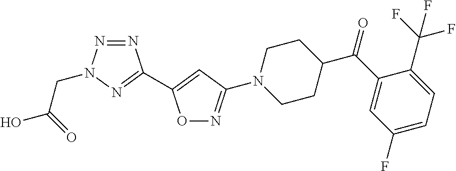 | 469 (M + 1) |
| 10 | 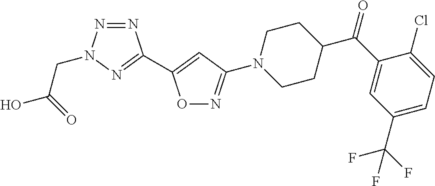 | 485 (M + 1) |
| 15 | 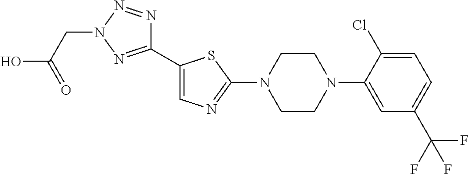 | 474 (M + 1) |
| 16 | 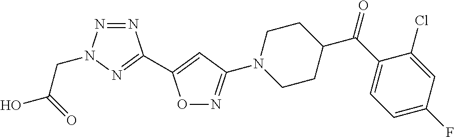 | 435 (M + 1) |
| 16 | 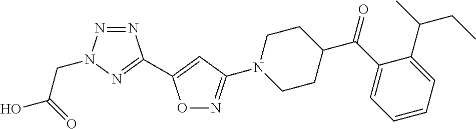 | 439 (M + 1) |
| 16 | 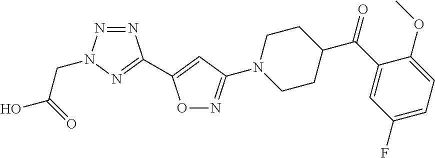 | 431 (M + 1) |
| 16 | 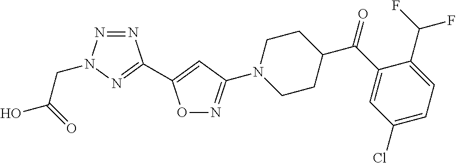 | 467 (M + 1) |
| 16 | 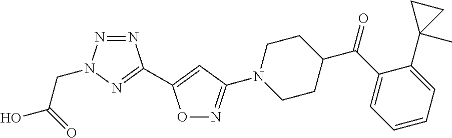 | 437 (M + 1) |
| 16 | 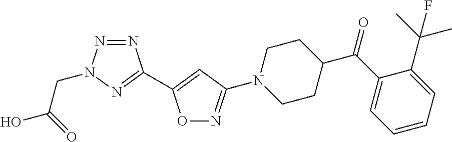 | 443 (M + 1) |
| 16 | 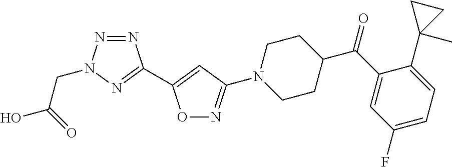 | 455 (M + 1) |
| 16 | 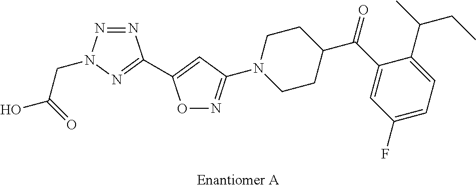 | 457 (M + 1) |
| 16 | 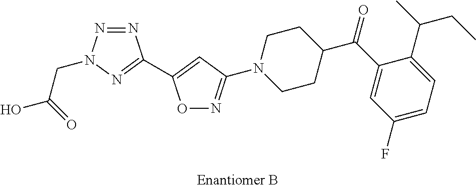 | 457 (M + 1) |
| 16 | 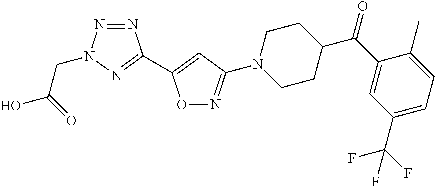 | 465 (M + 1) |
| 16 | 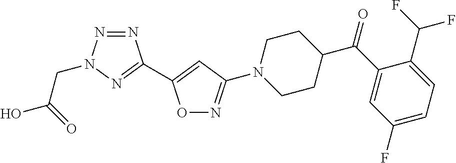 | 451 (M + 1) |
| 16 | 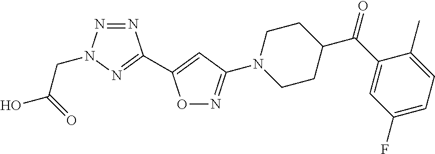 | 415 (M + 1) |
| 16 | 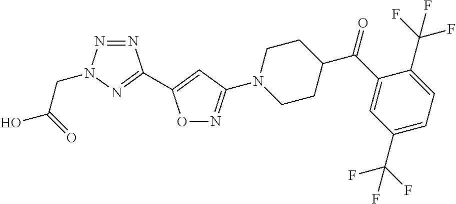 | 519 (M + 1) |
| 16 | 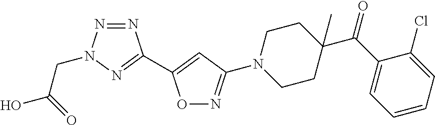 | 431 (M + 1) |
| 16 | 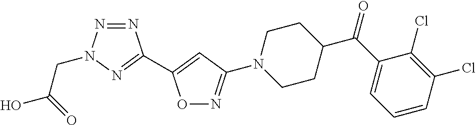 | 451 (M + 1) |
| 16 | 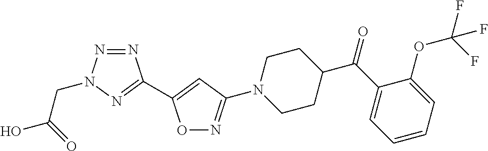 | 467 (M + 1) |
| 14 | 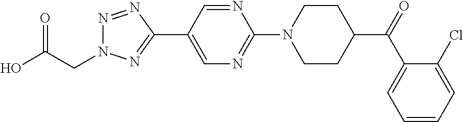 | 428 (M + 1) |
| 14 | 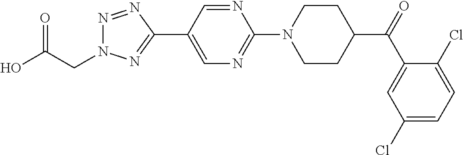 | 462 (M + 1) |
| 14 | 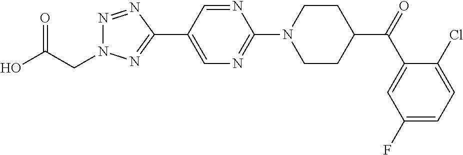 | 446 (M + 1) |
| 14 | 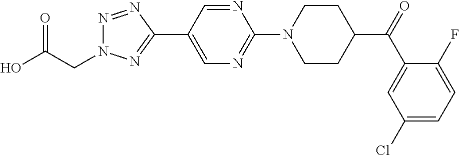 | 446 (M + 1) |
| 14 | 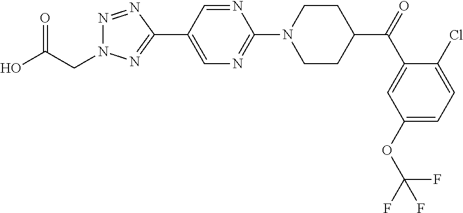 | 512 (M + 1) |
| 14 | 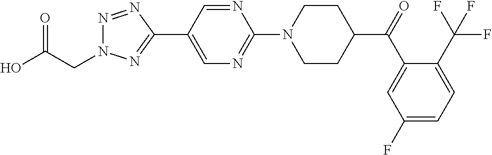 | 480 (M + 1) |
| 14 | 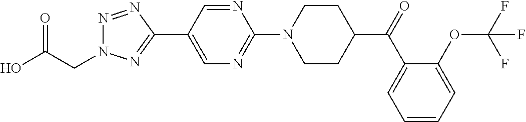 | 478 (M + 1) |
| 14 | 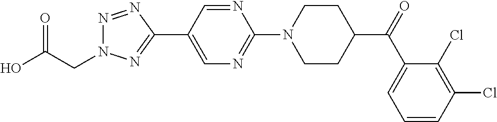 | 462 (M + 1) |
| 16 | 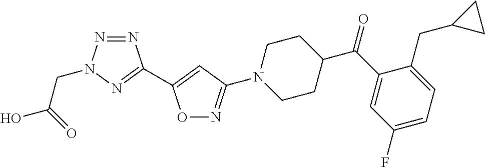 | 455 (M + 1) |
| 16 | 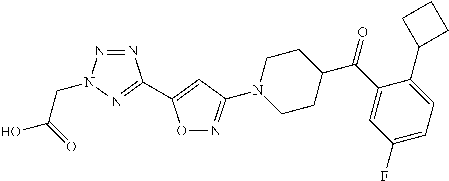 | 455 (M + 1) |
| 16 | 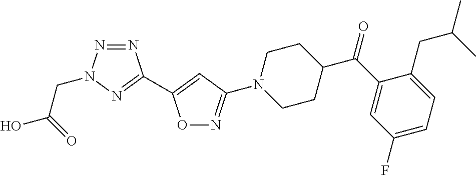 | 457 (M + 1) |
| 16 | 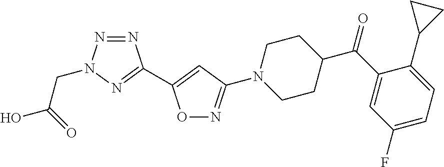 | 441 (M + 1) |
| 16 | 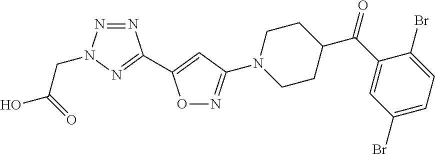 | 541 (M + 1) |
| 10 | 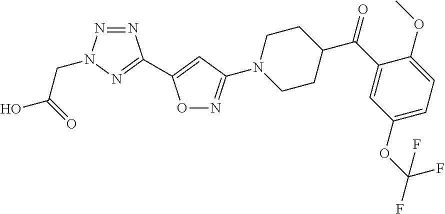 | 497 (M + 1) |
| 16 | 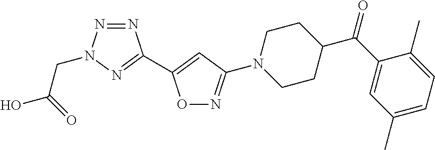 | 411 (M + 1) |
| 16 | 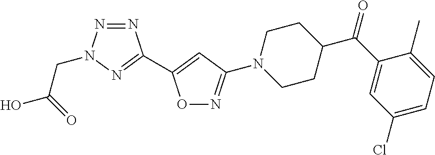 | 431 (M + 1) |
| 16 | 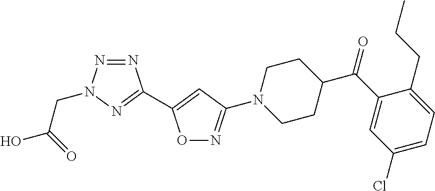 | 459 (M + 1) |
| 16 | 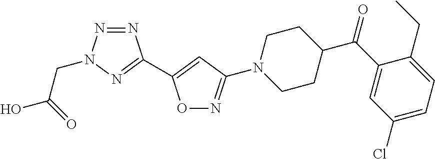 | 445 (M + 1) |
| 16 | 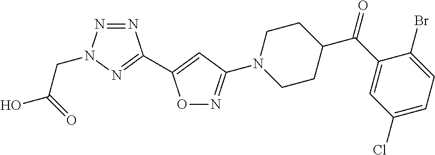 | 495, 497 (M + 1) |
| 10 | 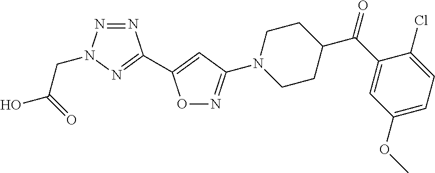 | 447 (M + 1) |
| 10 | 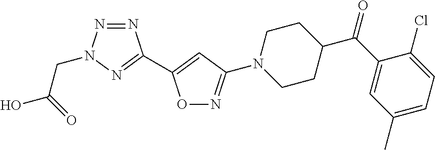 | 431 (M + 1) |
| 16 | 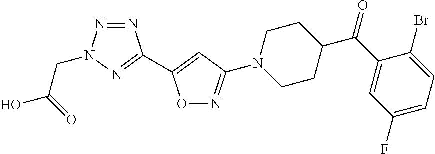 | 479, 481 (M + 1) |
| 10 | 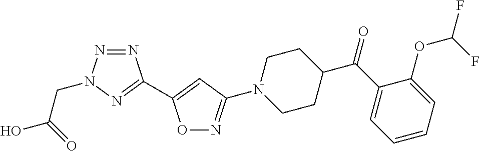 | 449 (M + 1) |
| 16 | 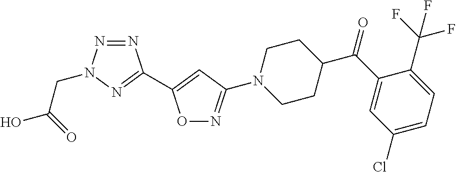 | 485 (M + 1) |
| 16 | 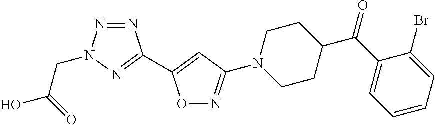 | 461, 463 (M + 1) |
| 10 | 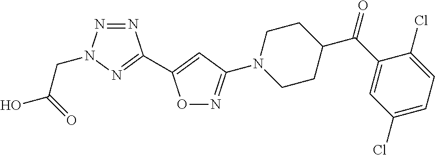 | 451 (M + 1) |
| 10 | 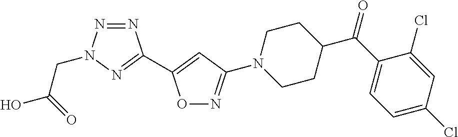 | 451 (M + 1) |

### Examples of Pharmaceutical Formulations

As a specific embodiment of an oral composition of a compound of the present invention, 50 mg of the compound of any of the Examples is formulated with sufficient finely divided lactose to provide a total amount of 580 to 590 mg to fill a size 0 hard gelatin capsule.

As a second specific embodiment of an oral pharmaceutical composition, a 100 mg potency tablet is composed of 100 mg of any one of the Examples, 268 mg microcrystalline cellulose, 20 mg of croscarmellose sodium, and 4 mg of magnesium stearate. The active, microcrystalline cellulose, and croscarmellose are blended first. The mixture is then lubricated by magnesium stearate and pressed into tablets.

While the invention has been described and illustrated in reference to specific embodiments thereof, those skilled in the art will appreciate that various changes, modifications, and substitutions can be made therein without departing from the spirit and scope of the invention. For example, effective dosages other than the preferred doses as set forth hereinabove may be applicable as a consequence of variations in the responsiveness of the human being treated for a particular condition. Likewise, the pharmacologic response observed may vary according to and depending upon the particular active compound selected or whether there are present pharmaceutical carriers, as well as the type of formulation and mode of administration employed, and such expected variations or differences in the results are contemplated in accordance with the objects and practices of the present invention. It is intended therefore that the invention be limited only by the scope of the claims which follow and that such claims be interpreted as broadly as is reasonable.

1. A compound of structural formula I: 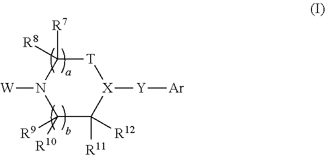
or a pharmaceutically acceptable salt thereof; wherein
“a” is an integer selected from 0, 1, and 2;
“b” is an integer selected from 0, 1, and 2;
with the proviso that “a” and “b” cannot both be 2;
X-T is N—CR⁵R⁶, C═CR⁵, or CR¹³—CR⁵R⁶;
Y is a bond or C(═O);
W is heteroaryl selected from the group consisting of: 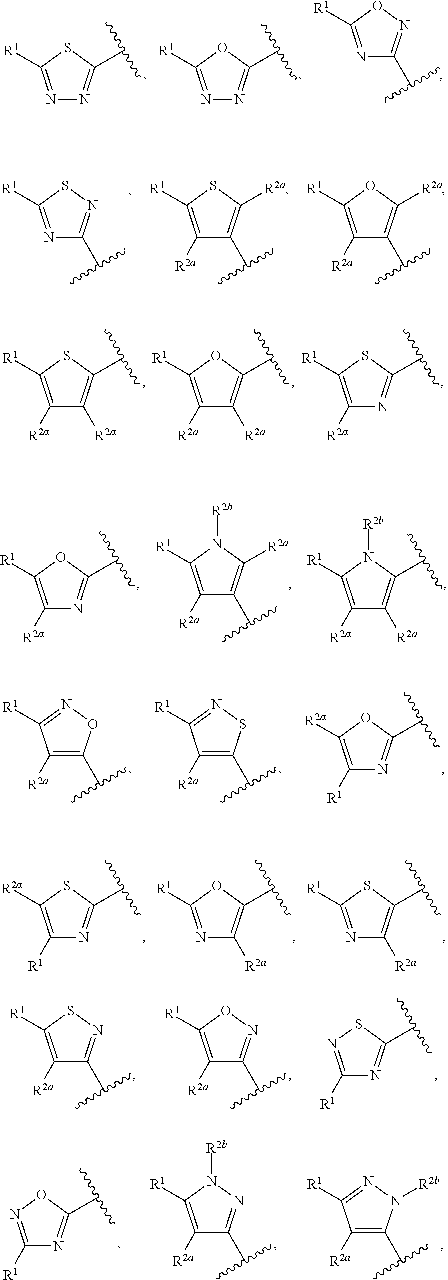 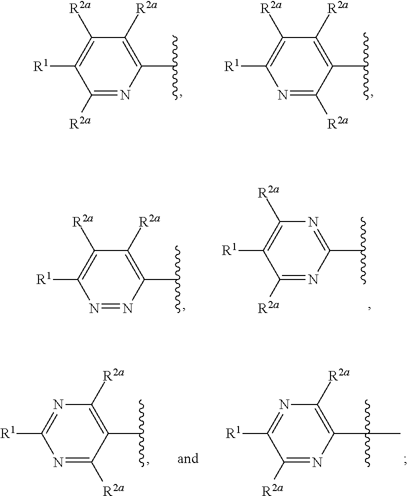
R¹ is heteroaryl selected from the group consisting of: 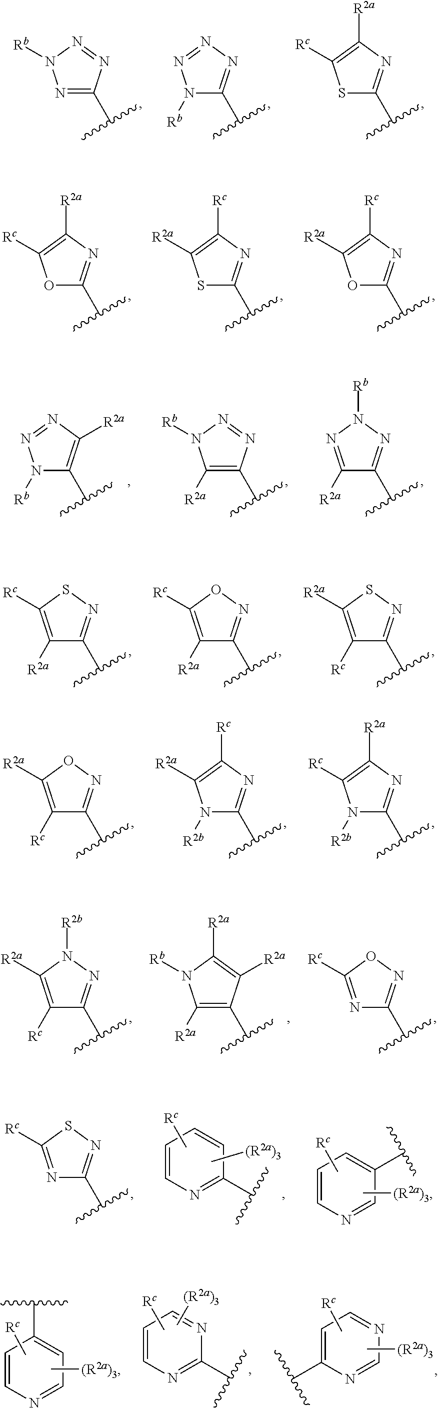 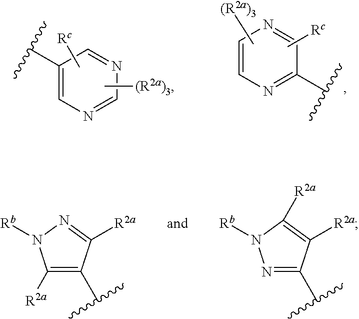
wherein
R^{b} is —(CH₂)ᵣCO₂H, —(CH₂)ᵣCO₂C₁₋₃ alkyl, —(CH₂)ᵣ—Z—(CH₂)ₚCO₂H, or —(CH₂)ᵣ—Z—(CH₂)ₚCO₂C₁₋₃ alkyl;
R^{c} is —(CH₂)ₘCO₂H, —(CH₂)ₘCO₂C₁₋₃ alkyl, —(CH₂)ₘ—Z—(CH₂)ₚCO₂H, or —(CH₂)ₘ—Z—(CH₂)ₚCO₂C₁₋₃ alkyl;
Z is O, S, or NR⁴;
each R^{2a} is independently selected from the group consisting of:
hydrogen,
halogen,
hydroxy,
cyano,
amino,
C₁₋₄ alkyl, optionally substituted with one to five fluorines,
C₁₋₄ alkoxy, optionally substituted with one to five fluorines,
C₁₋₄ alkylthio, optionally substituted with one to five fluorines,
C₁₋₄ alkylsulfonyl, optionally substituted with one to five fluorines,
carboxy,
C₁₋₄ alkyloxycarbonyl, and
C₁₋₄ alkylcarbonyl;
each R^{2b} is independently selected from the group consisting of:
hydrogen,
C₁₋₄ alkyl, optionally substituted with one to five fluorines,
C₁₋₄ alkylsulfonyl, optionally substituted with one to five fluorines,
C₁₋₄ alkyloxycarbonyl, and
C₁₋₄ alkylcarbonyl;
Ar is phenyl, naphthyl, thienyl, or pyridyl optionally substituted with one to five R³ substituents;
each R³ is independently selected from the group consisting of:
halogen,
cyano,
C₁₋₆ alkyl, optionally substituted with one to five fluorines,
C₁₋₆ alkoxy, optionally substituted with one to five fluorines,
C₁₋₆ alkylthio, optionally substituted with one to five fluorines, and
C₁₋₆ alkylsulfonyl, optionally substituted with one to five fluorines;
each R⁴ is independently selected from the group consisting of
hydrogen,
C₁₋₆ alkyl,
(CH₂)ₙ-phenyl,
(CH₂)ₙ-heteroaryl,
(CH₂)ₙ-naphthyl, and
(CH₂)ₙC₃₋₇ cycloalkyl;
wherein alkyl, phenyl, heteroaryl, naphthyl, and cycloalkyl are optionally substituted with one to three groups independently selected from halogen, C₁₋₄ alkyl, and C₁₋₄ alkoxy;
R⁵, R⁶, R⁷, R⁸, R⁹, R¹⁰, R¹¹, and R¹² are each independently hydrogen, fluorine, or C₁₋₃ alkyl,
wherein alkyl is optionally substituted with one to three substituents independently selected from fluorine and hydroxy;
or one of R⁵, R⁶, R⁷, and R⁸ together with one of R⁹, R¹⁰, R¹¹, and R¹² forms a direct bond or a C₁₋₂ alkylene bridge;
R¹³ is hydrogen, C₁₋₃ alkyl, fluorine, or hydroxy;
m is an integer from 0 to 3;
n is an integer from 0 to 2;
p is an integer from 1 to 3; and
r is an integer from 1 to 3.

2. The compound of claim 1 wherein “a” and “b” are each 1.

3. The compound of claim 2 wherein X-T is CR¹³—CR⁵R⁶; and Y is a bond.

4. The compound of claim 2 wherein X-T is CR¹³—CR⁵R⁶; and Y is C(═O).

5. The compound of claim 2 wherein X-T is N—CR⁵R⁶; and Y is a bond.

6. The compound of claim 5 wherein one of R⁵, R⁶, R⁷, and R⁸ together with one of R⁹, R¹⁰, R¹¹, and R¹² forms a methylene bridge.

7. The compound of claim 2 wherein X-T is N—CR⁵R⁶; and Y is C(═O).

8. The compound of claim 2 wherein X-T is C═CR⁵; and Y is a bond.

9. The compound of claim 1 wherein “a” is 1 and “b” is 2.

10. The compound of claim 9 wherein X-T is N—CR⁵R⁶; and Y is a bond.

11. The compound of claim 1 wherein Ar is phenyl optionally substituted with one to three substituents independently selected from R³.

12. The compound of claim 1 wherein R⁵, R⁶, R⁷, R⁸, R⁹, R¹⁰, R¹¹, R¹² and R¹³ are each hydrogen.

13. (canceled)

14. The compound of claim 1 wherein W is heteroaryl selected from the group consisting of:

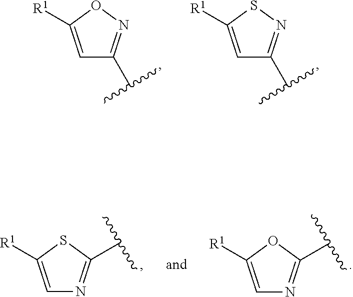

15. (canceled)

16. The compound of claim 1 wherein W is

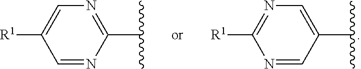

17. The compound of claim 1 wherein R¹ is heteroaryl selected from the group consisting of: 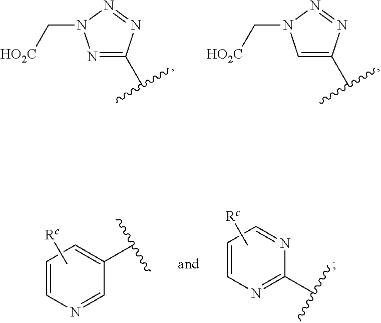
wherein R^{c} is —CO₂H, —CO₂C₁₋₃ alkyl, —CH₂CO₂H, or —CH₂CO₂C₁₋₃ alkyl.

18. The compound of claim 17 wherein R¹ is

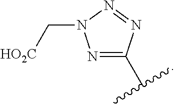

19-24. (canceled)

25. A compound selected from the group consisting of: 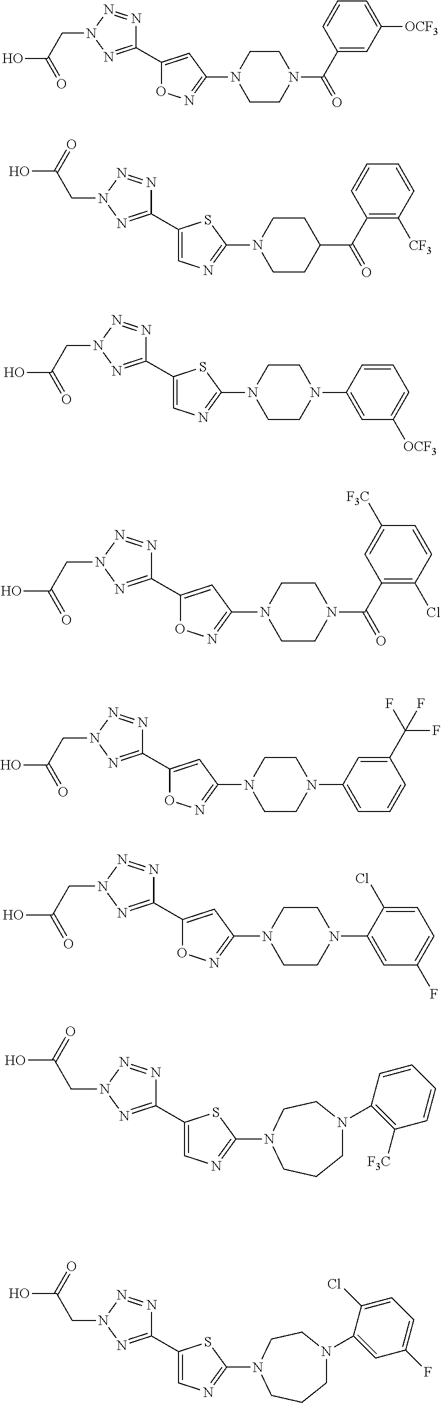 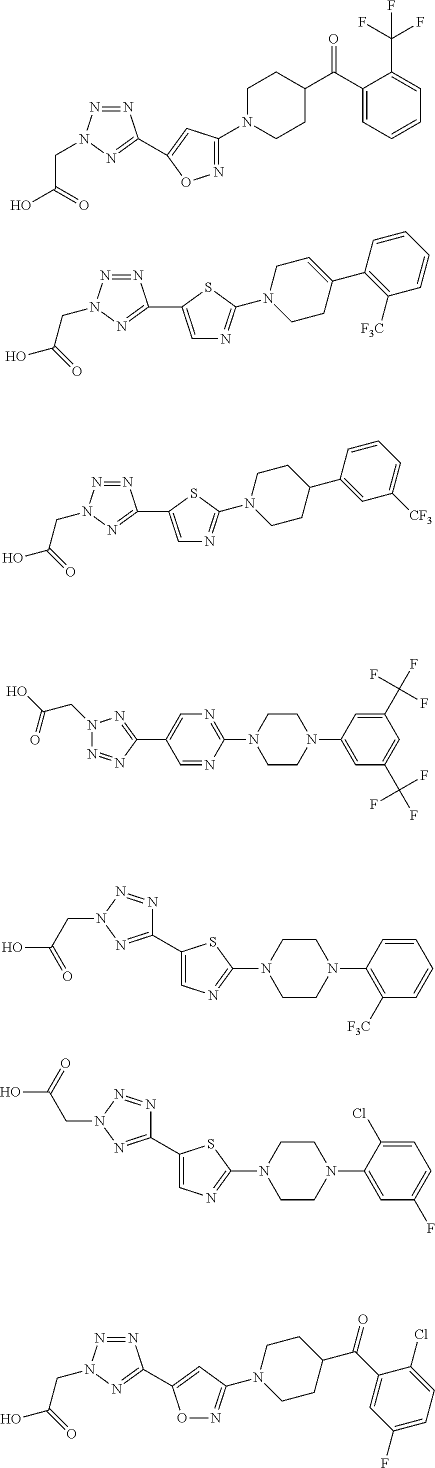 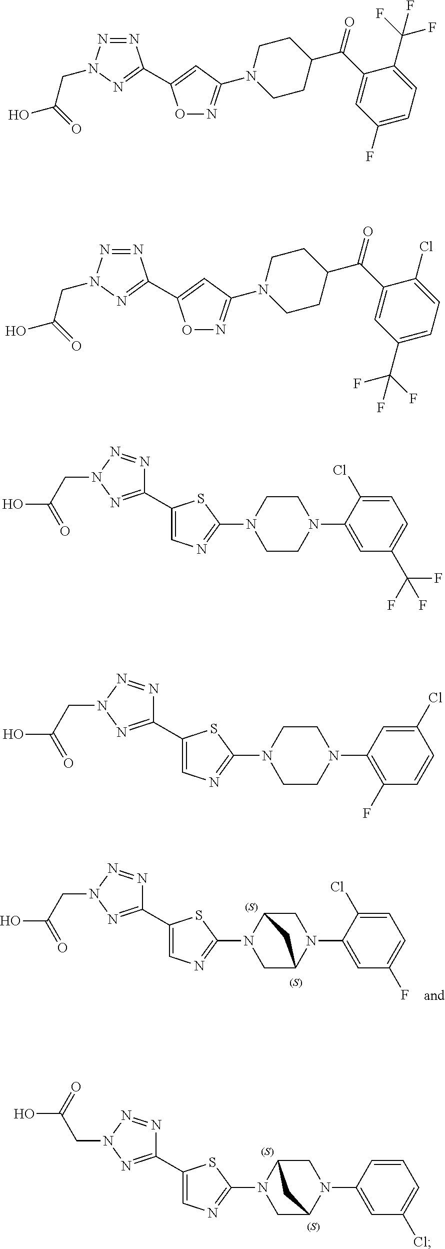
or a pharmaceutically acceptable salt thereof.

26. A pharmaceutical composition comprising a compound in accordance with claim 1 in combination with a pharmaceutically acceptable carrier.

27-31. (canceled)

32. A method of treating hyperglycemia, diabetes or insulin resistance in a mammal in need thereof which comprises the administration to the mammal of a therapeutically effective amount of a compound of claim 1.

33. A method of treating a lipid disorder selected from the group consisting of dyslipidemia, hyperlipidemia, hypertriglyceridemia, hypercholesterolemia, low HDL, and high LDL in a mammal in need thereof which comprises the administration to the mammal of a therapeutically effective amount of a compound of claim 1.
